(19) United States
(12) Patent Application Publication
(10) Pub. No.: US 2012/0136369 A1
(43) Pub. Date: May 31, 2012

(54) STRUCTURES FOR OCCLUDING THE UTERINE ARTERY

(75) Inventors: Yossi GROSS, Moshav Mazor (IL); Eliahu ELIACHAR, Haifa (IL); Dan SADE HOCHSTADTER, Kibbutz Bet Alfa (IL); Jacob COHEN, Ramat HaSharon (IL); Herman WEISS, Beit Shemesh (IL); Nir LILACH, K'far Yehoshua (IL)

(73) Assignee: FIBRO CONTROL, INC.,, Wilmington, DE (US)

(21) Appl. No.: 13/352,894

(22) Filed: Jan. 18, 2012

Related U.S. Application Data
(63) Continuation-in-part of application No. PCT/IL2010/000610, filed on Jul. 27, 2010.
Application No. PCT/IL2010/000610 is a continuation-in-part of application No. 12/509,732, filed on Jul. 27, 2009.

(51) Int. Cl. A61B 17/42

(52) U.S. Cl. 606/119

(57) ABSTRACT

Apparatus and methods are described including at least one rigid structure configured to be inserted into a subject's fornix such that distal end of the rigid structure is at a site anterior to a perivascular adipose layer of at least one of the subject's left and right broad ligaments. At least one uterine artery compression device is placed posterior to the broad ligaments and compresses at least one of the subject's left and right uterine arteries by pressing the at least one broad ligament against the rigid structure. Other applications are also described.

### CROSS-REFERENCES TO RELATED APPLICATIONS

The present application is a continuation-in-part of PCT Application PCT/IL2010/000610 to Gross (published as WO 11/013,127), filed Jul. 27, 2010, entitled “Structures for occluding the uterine artery,” which:

claims priority from and is a continuation-in-part of U.S. patent application Ser. No. 12/509,732 to Gross (published as 2011/0022073), filed Jul. 27, 2009, entitled “Balloon with rigid rod for occluding the uterine artery;” and

claims priority from U.S. 61/300,262 to Gross, filed Feb. 1, 2010, entitled “Non-penetrating structures for occluding uterine arteries.”

The present patent application is related to U.S. patent application Ser. No. 12/374,884 to Gross (published as US 2009/0318950), which is the US national phase of PCT Application PCT/IL2007/000911 to Gross (published as WO 08/012,802), filed Jul. 18, 2007, entitled, “Fibroid treatment apparatus and method,” which is a continuation-in-part of U.S. patent application Ser. No. 11/591,044 to Gross (published as US 2009/0093758), filed Oct. 31, 2006, entitled, “Fibroid treatment apparatus and method,” which claims priority from U.S. Provisional Patent Application 60/820,130 to Gross, filed Jul. 24, 2006, entitled, “Fibroid treatment apparatus and method.”

All of the above-referenced applications are incorporated herein by reference.

### FIELD OF EMBODIMENTS OF THE INVENTION

Applications of the present invention generally relate to medical apparatus. Specifically, applications of the present invention relate to apparatus and methods for occluding a subject's uterine arteries.

### BACKGROUND

Uterine fibroids are benign tumors of muscle and connective tissue that develop within, or are attached to, the uterine wall.

### SUMMARY OF EMBODIMENTS

For some applications of the present invention, a uterine artery compression device is used to occlude one or both of a subject's uterine arteries. The occlusion of the uterine artery is typically performed in order to reduce the supply of blood to a uterine fibroid, thereby causing irreversible ischemic necrosis and death of the fibroid.

For some applications, a fornix-engaging structure (e.g., a cervix cap) is inserted into the subject's vagina such that the structure engages the subject's vaginal fornix. First and second uterine artery compression devices, for example, occluding balloons, are inserted into the subject's body, via the fornix-engaging structure. The balloons are placed at sites outside of but in a vicinity of the left and right uterine arteries, respectively. The balloons are typically placed at sites that are posterior to the subject's broad ligament. When the balloons are at the sites, the balloons compress, and at least partially occlude, the uterine arteries by being inflated.

Typically prior to the occluding step, a positioning-anchoring balloon, disposed on the end of a positioning-anchoring tube, is inserted into the subject's uterus and inflated, such that the positioning-anchoring balloon stabilizes the occluding balloons. A blood flow monitoring system is used to monitor blood flow through the uterine arteries. For example, oximeters of the blood flow monitoring system may be disposed on the positioning-anchoring tube and used to detect when blood flow through the uterine arteries has stopped (e.g., by detecting the presence of a pulse), by detecting a level of oxyhemoglobin in the vicinity of the uterine arteries (e.g., in the subject's cervix and/or fornix).

For some applications, at least one rigid structure is inserted into left and right extrauterine positions anterior to at least one of the left and right broad ligaments. Typically, first and second rigid structures are inserted into left and right extrauterine positions anterior to the left and right broad ligaments. The left and right uterine artery compression devices compress the left and right uterine arteries by squeezing tissue against, respectively, the first and second rigid structures. For some applications, a single uterine artery compression device is placed at a site that is posterior to the broad ligament and the single uterine artery compression device squeezes both the left and right uterine arteries, respectively, against the left and right rigid structures.

For some applications, the compression device is coupled to the distal end of a rigid rod, which is typically hollow (i.e., it is a rigid tube), but for some applications is solid. Although some specific embodiments are described herein with respect to a rigid tube, it is noted that this is by way of illustration and not limitation, and the scope of the present invention includes use of a solid rod instead, unless context or explicit statement indicates otherwise. For example, the compression device may be a balloon that is disposed around the distal end of the rigid tube. The tube is inserted into the subject's body such that the distal end of the tube passes through vaginal tissue until the distal end of the tube is at a site (“the compression site”) outside of the uterine artery of the subject, but in a vicinity of a portion of the uterine artery that supplies the uterine fibroid. When the distal end of the tube is at the site, the compression device compresses the artery. For example, the balloon is inflated such that the balloon compresses the artery. The rigidity of the tube typically ensures that the position of the distal end of the tube is maintained at the compression site during the compression of the artery. In this manner, as the balloon is inflated, the balloon exerts pressure on the uterine artery, rather than pushing the distal end of the tube away from the compression site.

Typically a fornix-engaging structure is inserted into the subject's vagina such that the structure engages the subject's fornix. The tube is inserted through the vaginal tissue, and the distal end of the tube is positioned at the compression site using a tube-guide that is coupled to the fornix-engaging structure. For some applications, the tube-guide is configured such that the tube is inserted into the subject's body at an angle that is not parallel to the longitudinal axis of the fornix-engaging structure. Typically, inserting the tube at such an angle facilitates placement of the distal end of the tube at the compression site. For some applications, the tube-guide is configured such that the tube is inserted into the subject's body parallel to the longitudinal axis of the fornix-engaging structure.

For some applications, left and right balloons are used as the uterine artery compression device. The balloons are typically inserted into the subject's body, via an incision in the subject's posterior vaginal fornix. Further typically, the balloons are inserted while the balloons are (a) in deflated states, and (b) both inside of a cover. Typically, the balloons are disposed adjacent to one another, inside the cover, or at another suitable relative disposition with respect to one another. The balloons are inserted into a position that is posterior to the subject's cervix. When the balloons are in this position, the cover is withdrawn from around the balloons. Subsequently, the balloons are separated from each other, while the balloons are still in deflated states. The balloons are separated, such that after the balloons have been separated from each other, the balloons are positioned posterior to, respectively, the left and right broad ligaments. Typically, the separation of the balloons is achieved using a mechanical mechanism that simultaneously separates the two balloons and places them posterior to the left and right broad ligaments.

For some applications of the present invention, left and right rigid structures are inserted to the anterior side of the subject's fornix such that distal ends of the respective rigid structures are at sites anterior to, respectively, the subject's left and right broad ligaments. For some applications, the left and right rigid structures are placed at the sites anterior to the subject's broad ligaments without penetrating (or otherwise passing through) vaginal or any other tissue of the subject. A uterine artery compression device (e.g., the left and right balloons described hereinabove) is placed posterior to the broad ligaments (e.g., as described hereinabove). The uterine artery compression device compresses the subject's left and right uterine arteries by pressing the left and right broad ligaments against, respectively, the left and right rigid structures. In applications in which balloons, such as the left and right balloons described hereinabove, are used as the uterine artery compression device, the balloons (a) are positioned posterior to, respectively, the left and right broad ligaments, as described hereinabove, and (b) are subsequently inflated, in order to press the broad ligaments against the rigid structures.

Typically, a fornix-engaging structure (e.g., a cervix cap) is inserted into the subject's vagina such that the structure engages the subject's vaginal fornix. The left and right rigid structures are inserted via the fornix-engaging structure. For some applications, the uterine artery compression device is inserted through vaginal tissue via the fornix-engaging structure. For some applications, both the rigid structures and the uterine artery compression device are passed through the fornix-engaging structure.

For some applications, prior to the uterine artery compression device and the rigid structures having been inserted into their respective positions, a positioning-anchoring balloon, disposed on the end of a positioning-anchoring tube, is inserted into the subject's uterus, via the fornix-engaging structure. When the positioning-anchoring balloon is disposed inside the subject's uterus, the positioning-anchoring balloon is inflated. The positioning-anchoring balloon stabilizes the fornix-engaging structure, and thereby stabilizes the uterine artery compression device and the rigid structures, when the uterine artery compression device and the rigid structures are subsequently inserted via the fornix-engaging structure. For some applications, a fornix-engaging structure, a positioning-anchoring tube, and/or a positioning-anchoring rod are used, in accordance with the techniques described in US Patent application 2011/0022073 to Gross, which is incorporated herein by reference.

There is therefore provided, in accordance with some applications of the present invention, apparatus, for use with a body, vagina, vaginal fornix, uterus, cervix, left and right broad ligaments and left and right uterine arteries of a subject, the apparatus comprising:

at least one rigid structure configured to be inserted into the subject's fornix such that distal end of the rigid structure is at a site anterior to a perivascular adipose layer of at least one of the subject's left and right broad ligaments; and

at least one uterine artery compression device configured to be placed posterior to the broad ligaments and to compress at least one of the subject's left and right uterine arteries by pressing the at least one broad ligament against the rigid structure.

For some applications, the at least one rigid structure comprises left and right rigid structures configured to be inserted into the subject's fornix such that distal ends of the rigid structures are at sites anterior to perivascular adipose layers of, respectively, the subject's left and right broad ligaments, and the at least one uterine artery compression device is configured to compress the subject's left and right uterine arteries by pressing the subject's left and right broad ligaments against the rigid structures.

For some applications, the apparatus further includes left and right rigid-structure-support-elements, the left and right rigid structures being disposed, respectively, on the left and right rigid-structure-support-elements, the rigid-structure-support-elements being reversibly couplable to one another.

For some applications, the apparatus further includes:

at least one insertion assembly, the uterine artery compression device being disposed at a distal end of the insertion assembly; and

a fornix-engaging structure including first and second portions thereof,

the first portion of the fornix-engaging structure being coupled to the insertion assembly, and

the second portion of the fornix-engaging structure being configured to be couplable to the first portion of the fornix-engaging structure inside the subject's vagina.

For some applications, the uterine artery compression device includes a spring mechanism configured to facilitate expansion of the uterine artery compression device toward the subject's broad ligaments.

For some applications, the left and right rigid structures are configured not to pass through vaginal tissue of the subject.

For some applications, both the left and right rigid structures are configured to be inserted through a single incision in the subject's fornix.

For some applications, the left and right rigid structures are configured to be inserted through respective incisions in the subject's fornix.

For some applications, the apparatus further comprises an oximeter configured to be inserted via the subject's vagina and to measure a parameter of a portion of the subject's body selected from the group consisting of: the subject's uterus and the subject's cervix.

For some applications, the oximeter is configured to measure a level of oxyhemoglobin at the selected portion.

For some applications, the oximeter is configured to measure a pulse at the selected portion.

For some applications,

the apparatus further includes a fornix-engaging structure configured to be inserted into the vagina and to engage the vaginal fornix,

and the left and right rigid structures are configured to be inserted into the subject's fornix via the fornix-engaging structure.

For some applications, the apparatus further includes a locking-mechanism configured, subsequent to the insertion of the rigid structures via the fornix-engaging structure, to lock the rigid structures into a fixed position with respect to the fornix-engaging structure.

For some applications, the rigid structures include curved distal portions thereof.

For some applications, the rigid structures are not configured to incise tissue.

For some applications, a radius of curvature of each of the curved distal portions is between 1 mm and 20 mm.

For some applications, the radius of curvature of each of the curved distal portions is between 5 mm and 15 mm.

For some applications, the apparatus further includes:

at least one insertion assembly, the uterine artery compression device being disposed at a distal end of the insertion assembly; and

a fornix-engaging structure including first, second, and third portions thereof,

the second portion of the fornix-engaging structure being coupled to the insertion assembly,

the third portion of the fornix-engaging structure being coupled to the rigid structures, and

the first portion of the fornix-engaging structure being configured to be couplable to the second and third portions of the fornix-engaging structure inside the subject's vagina.

For some applications, the second and third portions of the fornix-engaging structure are shaped to engage a region of the first portion of the fornix-engaging structure such that motion of the first and second portion in a distal direction with respect to the first portion is prevented.

For some applications, the third portion of the fornix-engaging structure includes left and right rigid-structure-support-elements, the left and right rigid structures being disposed, respectively, on the left and right rigid-structure-support-elements, the rigid-structure-support-elements being reversibly couplable to one another.

For some applications, the apparatus further comprises an oximeter configured to be inserted via the subject's vagina and to measure a parameter of a portion of the subject's body selected from the group consisting of: the subject's uterus and the subject's cervix.

For some applications, the oximeter is configured to measure a level of oxyhemoglobin at the selected portion.

For some applications, the oximeter is configured to measure a pulse at the selected portion.

For some applications, the oximeter comprises an oximetry transmitter and an oximetry detector, and at least one of the oximetry receiver and the oximetry detector is coupled to the fornix-engaging structure.

For some applications, the uterine artery compression device includes left and right balloons configured to be placed at positions posterior to, respectively, the left and right broad ligaments.

For some applications, the apparatus further includes:

at least one connecting member, the balloons being coupled to one another via the connecting member; and

a rigid rod that is coupled to the balloons, and that is configured to maintain the balloons in a given configuration with respect to one another by flexing the connecting member.

For some applications, each of the balloons includes two chambers, the chambers being disposed with respect to one another, such that when the balloons are placed posterior to the broad ligaments, the balloons are configured to expand substantially in an anterior-posterior direction, upon inflation of the balloons.

For some applications, the apparatus further includes a measuring device configured to measure a parameter that is indicative of a level of blood-flow through the subject's uterine arteries, and a control unit configured to generate an output that is indicative of the level of blood flow through the uterine arteries.

For some applications, the measuring device includes a measuring device selected from the group consisting of an oximeter, a microphone, and a pressure sensor.

There is further provided, in accordance with some applications of the present invention, a method, for use with a body, vagina, vaginal fornix, uterus, cervix, left and right broad ligaments and left and right uterine arteries of a subject, the method including:

inserting at least one rigid structure into the subject's fornix such that distal end of the rigid structure is at a site anterior to a perivascular adipose layer of at least one of the subject's left and right broad ligaments;

placing at least one uterine artery compression device posterior to the broad ligaments; and

compressing at least one of the subject's left and right uterine arteries by pressing the at least one broad ligament against the rigid structures, using the uterine artery compression device.

For some applications, inserting the at least one rigid structure comprises inserting left and right rigid structures such that distal ends of the rigid structures are at sites anterior to perivascular adipose layers of, respectively, the subject's left and right broad ligaments, and compressing at least one of the subject's left and right uterine arteries comprises both of the subject's left and right uterine arteries by pressing the left and right broad ligaments against respective rigid structures, using the uterine artery compression device.

For some applications, the method further includes measuring a level of oxygen in the subject's uterus, and compressing the subject's uterine arteries includes compressing the uterine arteries responsively to the measured level of oxygen.

There is additionally provided, in accordance with some applications of the present invention, apparatus, for use with a body, vagina, vaginal fornix, uterus, cervix, left and right broad ligaments and left and right uterine arteries of a subject, the apparatus including:

at least one insertion assembly configured to be inserted into the subject's body, such that a distal end of the assembly passes through vaginal tissue until the distal end of the assembly is at a site posterior to the cervix; left and right balloons disposed on the distal end of the assembly; and

a balloon-placement mechanism configured to place the left and right balloons posterior to, respectively, the left and right broad ligaments, when the distal end of the insertion assembly is at the site posterior to the cervix.

For some applications, the apparatus further includes a fornix-engaging structure configured to be inserted into the vagina and to engage the vaginal fornix, and the insertion assembly is configured to be inserted via the fornix-engaging structure.

For some applications,

the apparatus further includes a fornix-engaging structure including first and second portions thereof,

the first portion of the fornix-engaging structure being coupled to the insertion assembly, and

the second portion of the fornix-engaging structure being configured to be couplable to the first portion of the fornix-engaging structure inside the subject's vagina.

For some applications, the balloon-placement mechanism is configured to place the left and right balloons posterior to the broad ligaments by simultaneously lifting the balloons anteriorly, and separating the balloons from one another.

For some applications,

the apparatus further includes left and right rigid structures configured to be inserted into the subject's fornix, such that distal ends of the rigid structures are at sites anterior to perivascular adipose layers of, respectively, the left and right broad ligaments,

and the left and right balloons are configured to compress the left and right uterine arteries by pressing the subject's broad ligaments against, respectively, the left and right rigid structures.

For some applications,

the apparatus further includes a fornix-engaging structure including first, second, and third portions thereof,
the second portion of the fornix-engaging structure being coupled to the insertion assembly,
the third portion of the fornix-engaging structure being coupled to the rigid structures, and
the first portion of the fornix-engaging structure being configured to be couplable to the second and third portions of the fornix-engaging structure inside the subject's vagina.

There is further provided in accordance with some applications of the present invention, a method, for use with a body, vagina, vaginal fornix, uterus, cervix, left and right broad ligaments and left and right uterine arteries of a subject, the method including:
inserting left and right balloons that are disposed at a distal end of an insertion assembly into the subject's body, such that the balloons pass through vaginal tissue until the balloons are at a site posterior to the cervix; and

placing the left and right balloons posterior to, respectively, the left and right broad ligaments, when the distal end of the insertion assembly is at the site posterior to the cervix, by actuating a mechanism.

There is additionally provided, in accordance with some applications of the present invention, apparatus, including:

left and right uterine artery compression devices configured to occlude, respectively, left and right uterine arteries of a subject; and

a flexible material that couples the left and right uterine artery compression devices to one another.

For some applications, the apparatus further includes a rigid rod that is coupled to the uterine artery compression devices, and that is configured to maintain the uterine artery compression devices in a given configuration by flexing the flexible material.

For some applications, the apparatus further includes at least one rigid structure configured to be placed at least in part in a vaginal fornix of the subject, and the uterine artery compression devices are configured to occlude the uterine arteries by pressing the uterine arteries against the rigid structure.

For some applications, the apparatus further includes:

a cover, the compression devices being configured to be inserted through vaginal tissue of the subject, while the compression devices are (a) in deflated states thereof and (b) both inside of the cover; and

a mechanism configured, subsequent to the compression devices having been inserted through the subject's vaginal tissue, to separate the compression devices from each other.

For some applications, the flexible material defines a maximum distance between centers of the left and right uterine artery compression devices of 25-200 mm.

For some applications, the flexible material defines a maximum distance between centers of the left and right uterine artery compression devices of 40-80 mm.

For some applications, the flexible material has a length of 5-60 mm.

There is further provided, in accordance with some applications of the present invention a method, including:

providing left and right uterine artery compression devices coupled to each other by a flexible material; and

occluding left and right uterine arteries of a subject using, respectively, the left and right uterine artery compression devices.

There is further provided, in accordance with some applications of the present invention, apparatus, for use with a subject's body, vagina, vaginal fornix, uterus, and uterine artery, the apparatus including:

a fornix-engaging structure configured to be inserted into the vagina and to engage the vaginal fornix;

a rod configured to be inserted into the subject's body via the fornix-engaging structure, such that a distal end of the rod passes through vaginal tissue at a first vaginal site until the distal end of the rod is at a first extrauterine site outside of the uterine artery, but in a vicinity of a portion of the uterine artery that supplies a uterine fibroid;

a rod-guide coupled to the fornix-engaging structure, and configured to guide the distal end of the rod to the first extrauterine site; and

a uterine artery compression device disposed on the distal end of the rod.

For some applications, the rod is a hollow rod.

For some applications, the rod is a solid rod.

For some applications, the rod is shaped to prevent rotation of the rod with respect to the rod-guide.

For some applications, the fornix-engaging structure defines a longitudinal axis thereof, and the rod-guide is shaped to define a hole for guiding the rod, a longitudinal axis of the hole being parallel to the longitudinal axis of the fornix-engaging structure.

For some applications, the apparatus further includes a blood flow sensor configured to detect a change in blood flow through the uterine artery.

For some applications, the apparatus further includes a positioning-anchoring rod and a positioning-anchoring balloon disposed at a distal end of the positioning-anchoring rod, and:

the distal end of the positioning-anchoring rod is configured to be inserted into the subject's uterus, via a cervix of the subject, and

the positioning-anchoring balloon is configured to anchor the uterine artery compression device while the uterine artery compression device is outside of the uterus, by the positioning-anchoring balloon being inflated while the distal end of the positioning-anchoring rod is inside the subject's uterus.

For some applications, the fornix-engaging structure and the rod-guide are coupled by being an integrated unit.

For some applications, the rod-guide is reversibly couplable to the fornix-engaging structure.

For some applications, the rod-guide is configured to be coupled to the fornix-engaging structure before the fornix-engaging structure is inserted into the subject's vagina.

For some applications, the rod-guide is configured to be coupled to the fornix-engaging structure when the fornix-engaging structure has engaged the vaginal fornix.

For some applications, the uterine artery compression device includes a balloon.

For some applications, the balloon is substantially not stretchable.

For some applications, the fornix-engaging structure defines a longitudinal axis thereof, and the rod-guide is shaped to define a hole for guiding the rod, a longitudinal axis of the hole not being parallel to the longitudinal axis of the fornix-engaging structure.

For some applications, an angle between the longitudinal axis of the rod-guide and the longitudinal axis of the hole is less than 60 degrees.

For some applications, the angle between the longitudinal axis of the rod-guide and the longitudinal axis of the hole is between 10 degrees and 45 degrees.

For some applications, the angle between the longitudinal axis of the rod-guide and the longitudinal axis of the hole is between 15 degrees and 30 degrees.

For some applications, the rod includes a rigid rod.

For some applications, the fornix-engaging structure and the rod-guide include rigid structures configured to maintain the distal end of the rod at the site by supporting the rod.

For some applications, the apparatus further includes a rigid positioning-anchoring rod and a positioning-anchoring balloon disposed at a distal end of the positioning-anchoring rod, and:

the distal end of the positioning-anchoring rod is configured to be inserted into the subject's uterus, via a cervix of the subject, and

the positioning-anchoring balloon is configured to anchor the uterine artery compression device while the uterine artery compression device is outside of the uterus, by the positioning-anchoring balloon being inflated while the distal end of the positioning-anchoring rod is inside the subject's uterus.

For some applications, the uterine artery compression device is configured to compress the uterine artery by squeezing tissue of the subject against a portion of the apparatus.

For some applications, the uterine artery compression device is configured to compress the uterine artery by squeezing the subject's tissue against the fornix-engaging structure.

For some applications:

the rod includes first and second rods configured to be inserted into the subject's body, such that distal ends of the rods are at sites outside of but in a vicinity of left and right uterine arteries of the subject, respectively, and

the uterine artery compression device includes left and right uterine artery compression devices disposed, respectively, on the distal end of the first and second rods, and configured to compress, respectively, the left and right uterine arteries by each uterine artery compression device squeezing tissue against the other uterine artery compression device.

For some applications, the apparatus further includes a positioning-anchoring rod and a positioning-anchoring balloon disposed at a distal end of the positioning-anchoring rod, and:

the distal end of the positioning-anchoring rod is configured to be inserted into the subject's uterus,

the positioning-anchoring balloon is configured to anchor the uterine artery compression device while the uterine artery compression device is outside of the uterus, by the positioning-anchoring balloon being inflated while the distal end of the rod is inside the subject's uterus, and

the uterine artery compression device is configured to compress the uterine artery by squeezing the subject's tissue against the positioning-anchoring rod.

For some applications, the apparatus further includes a positioning-anchoring rod and a positioning-anchoring balloon disposed at a distal end of the positioning-anchoring rod, and:

the distal end of the positioning-anchoring rod is configured to be inserted into the subject's uterus,

the positioning-anchoring balloon is configured to anchor the uterine artery compression device while the uterine artery compression device is outside of the uterus, by

the positioning-anchoring balloon being inflated while the distal end of the rod is inside the subject's uterus, and

the uterine artery compression device is configured to compress the uterine artery by squeezing the subject's tissue against the positioning-anchoring balloon.

For some applications:

the rod includes first and second rods configured to be inserted into the subject's body, such that distal ends of the rods are at left and right first extrauterine sites outside of but in a vicinity of left and right uterine arteries of the subject, respectively, and

the uterine artery compression device includes left and right uterine artery compression devices disposed, respectively, on the distal end of the first and second rods, and configured to compress, respectively, the left and right uterine arteries.

For some applications, the rod-guide is shaped to define at least one guiding portion at approximately a six o'clock position with respect to the subject's uterus, and is configured to guide the first and second rods through vaginal tissue at approximately the six o'clock position, via the at least one guiding portion.

For some applications, the rod-guide includes a first guiding portion configured to guide the distal end of the first rod to the left first extrauterine site, and a second guiding portion configured to guide the distal end of the second rod to the right first extrauterine site, and an angle defined by the first guiding portion of the rod-guide, a longitudinal axis of the rod-guide, and the second guiding portion of the rod-guide is less than 10 degrees.

For some applications,

the rod-guide is shaped to define first and second guiding portions at approximately a three o'clock position and a nine o'clock position with respect to the uterus,

the rod-guide is configured to guide the first rod through vaginal tissue at approximately the three o'clock position, via the first guiding portion, and

the rod-guide is configured to guide the second rod through vaginal tissue at approximately the nine o'clock position, via the second guiding portion.

For some applications, the rod-guide includes a first guiding portion configured to guide the distal end of the first rod to the left first extrauterine site, and a second guiding portion configured to guide the distal end of the second rod to the right first extrauterine site, and an angle defined by the first guiding portion of the rod-guide, a longitudinal axis of the rod-guide, and the second guiding portion of the rod-guide is between 170 and 190 degrees.

For some applications,

the apparatus further includes a rigid structure configured to be inserted into the subject's body via the fornix-engaging structure, such that a distal end of the rigid structure passes through vaginal tissue at a second vaginal site, until the distal end of the rigid structure is at a second extrauterine site outside of the uterine artery,

and the uterine artery compression device is configured to compress the uterine artery by squeezing tissue of the subject against the rigid structure.

For some applications, the rigid structure includes a curved distal portion thereof.

For some applications, a distal portion of the rigid structure is substantially straight.

For some applications, the rod and the rigid structure are pivotally connected to each other.

For some applications:

the rod includes first and second rods configured to be inserted into the subject's body, such that distal ends of the rods are at a first pair of extrauterine sites outside of but in a vicinity of left and right uterine arteries of the subject, respectively,

the uterine artery compression device includes left and right uterine artery compression devices disposed, respectively, on the distal end of the first and second rods,

the rigid structure includes left and right rigid structures configured to be inserted into the subject's body via the fornix-engaging structure, such that the distal ends of the rigid structures are at a second pair of extrauterine sites outside of but in a vicinity of left and right uterine arteries of the subject, respectively,

the left uterine artery compression device is configured to compress the left uterine artery by squeezing tissue of the subject against the left rigid structure, and

the right uterine artery compression device is configured to compress the right uterine artery by squeezing tissue of the subject against the right rigid structure.

For some applications:

the rigid structure includes left and right rigid structures configured to be inserted into the subject's body via the fornix-engaging structure, such that the distal ends of the rigid structures are at second extrauterine sites outside of but in a vicinity of left and right uterine arteries of the subject, respectively, and

the uterine artery compression device includes a single uterine artery compression device that is configured to:
compress the left uterine artery by squeezing tissue of the subject against the left rigid structure, and
compress the right uterine artery by squeezing tissue of the subject against the right rigid structure.

For some applications, the rod-guide includes a first guiding portion configured to guide the distal end of the rod to the first extrauterine site, and a second guiding portion configured to guide the distal end of the rigid structure to the second extrauterine site, and an angle defined by the first guiding portion of the rod-guide, a longitudinal axis of the rod-guide, and the second guiding portion of the rod-guide is greater than 100 degrees.

For some applications, the rod-guide includes a first guiding portion configured to guide the distal end of the rod to a first extrauterine site that is posterior to a broad ligament of the subject, and a second guiding portion configured to guide the distal end of the rigid structure to a second extrauterine site that is anterior to a perivascular adipose layer of the broad ligament.

For some applications, the rigid structure includes a rigid solid rod.

For some applications, the rigid structure includes a rigid hollow rod.

For some applications, the apparatus further includes a balloon disposed on the distal end of the rigid structure.

For some applications, the balloon is substantially not stretchable.

For some applications:

the rod-guide defines at least one guiding portion at approximately a position with respect to the subject's uterus, selected from the group consisting of: a 2 o'clock position, a 3 o'clock position, a 6 o'clock position, a 9 o'clock position, and a 10 o'clock position, and

the rod-guide is configured to guide the rod and the rigid structure through vaginal tissue, via the at least one guiding portion.

For some applications:

the rod-guide includes (a) a first guiding portion at approximately a 6 o'clock position with respect to the subject's uterus, and (b) a second guiding portion at approximately a position with respect to the subject's uterus selected from the group consisting of: a 2 o'clock position and a 10 o'clock position,

the rod-guide is configured to guide the rod through vaginal tissue at approximately the 6 o'clock position, via the first guiding portion, and

the rod-guide is configured to guide the rigid structure through vaginal tissue at approximately the selected position, via the second guiding portion.

For some applications:

the rod-guide defines a first guiding portion at approximately a 2 o'clock position with respect to the subject's uterus, a second guiding portion at approximately a 10 o'clock position with respect to the subject's uterus, and a third guiding portion at approximately a 6 o'clock position with respect to the subject's uterus,

the rigid structure includes left and right rigid structures,

the rod-guide is configured to guide the left rigid structure through vaginal tissue at approximately the 2 o'clock position, via the first guiding portion,

the rod-guide is configured to guide the right rigid structure through vaginal tissue at approximately the 10 o'clock position, via the second guiding portion, and

the rod-guide is configured to guide the rod through vaginal tissue at approximately the 6 o'clock position, via the third guiding portion.

There is further provided, in accordance with some applications of the present invention, apparatus, including:

a rod having a telescoping distal portion thereof, and a longitudinal axis thereof; and

a balloon disposed around the telescoping portion,

the apparatus being such that when the balloon is in an inflated state thereof, the rod is configured to increase a diameter of the balloon in a direction that is perpendicular to the longitudinal axis of the rod, by the telescoping portion of the rod being shortened.

For some applications, the rod is a hollow rod.

For some applications, the rod is a solid rod.

For some applications, the rod is configured to be inserted into a subject's body, such that a distal end of the rod passes through vaginal tissue, until the distal portion of the rod is at a site outside of a uterine artery of the subject, but in a vicinity of a portion of the uterine artery that supplies a uterine fibroid.

For some applications, the balloon is configured to at least partially occlude the uterine artery by being inflated while the distal portion is at the site.

For some applications, the balloon is configured to further occlude the uterine artery by the telescoping portion of the rod being shortened while the distal portion is at the site.

There is additionally provided, in accordance with some applications of the present invention, apparatus, including:

a measuring device configured to measure a parameter that is indicative of a level of blood-flow through blood vessels of a subject;

an output unit; and

a control unit configured to drive the output unit to generate an output that is (a) indicative of blood flow through a portion of one of the blood vessels having stopped due to (b) the parameter having changed and then plateaued at a value indicating non-zero blood flow in a vicinity of the blood vessels.

For some applications, the measuring device is configured to be placed in a vicinity of a uterine artery of the subject, and the control unit is configured to drive the output unit to generate an output that is (a) indicative of blood flow through a portion of the uterine artery having stopped due to (b) the parameter having changed and then plateaued at a value indicating non-zero blood flow in the vicinity of the uterine artery.

For some applications, the measuring device includes a pressure sensor configured to detect pressure in the vicinity of the blood vessels, and the control unit is configured to drive the output unit to generate an output that is indicative of a strength of a pulsating component of the detected pressure having decreased and then plateaued at a value indicating non-zero blood flow in the vicinity of the blood vessels.

For some applications, the measuring device includes a microphone configured to detect sound waves in the vicinity of the blood vessels, and the control unit is configured to drive the output unit to generate an output that is indicative of a strength of a pulsating component of the detected sound waves having decreased and then plateaued at a value indicating non-zero blood flow in the vicinity of the blood vessels.

For some applications, the measuring device includes a spectrometer.

For some applications, the measuring device includes an oximeter.

There is further provided, in accordance with some applications of the present invention, apparatus for use with a subject's uterus, including:

a rod, a distal end of which is configured to be inserted into the subject's uterus;

a balloon disposed on the distal end of the rod and configured to be inflated while the distal end of the rod is inside the subject's uterus; and

an oximeter disposed on the distal end of the rod and configured to detect a change in blood flow in a vicinity of the uterus by measuring a parameter in the vicinity while the distal end of the rod is inside the subject's uterus.

For some applications, the oximeter is configured to measure a level of oxyhemoglobin in the vicinity.

For some applications, the oximeter is configured to measure a pulse in the vicinity.

For some applications, the rod is hollow.

For some applications, the rod is solid.

There is further provided, in accordance with some applications of the present invention, apparatus, including:

a medical tool configured to be placed inside a body of a subject, outside of a reproductive tract of the subject; and

a positioning-anchoring balloon coupled to the tool, and configured to stabilize the tool by being inserted into a uterus of the subject and engaging the subject's uterus by the balloon being inflated.

For some applications, the medical tool includes a uterine artery compression device.

There is additionally provided, in accordance with some applications of the present invention, apparatus, for use with a subject's body, uterus, vagina and uterine artery, the apparatus including:

a guide structure having a first guiding portion at a site that is at approximately a 6 o'clock position with respect to the subject's uterus, and a second guiding portion that is approximately at a position with respect to the subject's uterus selected from the group consisting of: a 2 o'clock position and a 10 o'clock position,

a first compression device, the guide structure being configured to guide the first compression device into the subject's body, via a vaginal site of the vagina that is at approximately the 6 o'clock position, via the first guiding portion, and

a second compression device, the guide structure being configured to guide the second compression device into the subject's body, via a vaginal site of the vagina that is at approximately at the selected position, via the second guiding portion

the first and second compression devices being configured to compress the uterine artery by each compression device squeezing tissue against the other compression device.

There is further provided, in accordance with some applications of the present invention, apparatus, for use with a subject's vagina and uterine artery, the apparatus including:

first and second balloons configured to:

be inserted into the subject's body, via at least one vaginal site of the vagina, and

compress the uterine artery by each balloon squeezing tissue against the other balloon.

For some applications, one of the first and second balloons is substantially not stretchable, and another one of the first and second balloons is substantially stretchable.

For some applications, the apparatus further includes a pressure sensor configured to detect pressure in a vicinity of the balloons by detecting pressure inside the balloon that is substantially not stretchable.

There is further provided, in accordance with some applications of the present invention, a method, for use with a subject's body, vagina, vaginal fornix, uterus, and uterine artery, the method including:

engaging the vaginal fornix with a fornix-engaging structure, by inserting the structure into the vagina;

inserting a rod into the subject's body via the fornix-engaging structure;

using a rod-guide, guiding the rod such that a distal end of the rod passes through vaginal tissue at a first vaginal site until the distal end of the rod is at a first extrauterine site outside of the uterine artery, but in a vicinity of a portion of the uterine artery that supplies a uterine fibroid; and

while the distal end of the rod is at the site, compressing the uterine artery using a uterine artery compression device disposed on the distal end of the rod.

For some applications, the method further includes positioning and anchoring the uterine artery compression device while the uterine artery compression device is at the site, by placing a positioning-anchoring balloon inside the uterus and inflating the positioning-anchoring balloon while the positioning-anchoring balloon is inside the uterus.

For some applications, inserting the rod into the subject's body includes inserting a rigid rod into the subject's body, and compressing the uterine artery using the uterine artery compression device disposed on the distal end of the rod, includes maintaining the position of the distal end of the rod at the site, using the rigidity of the rod.

For some applications:

the rod includes left and right rods, and the uterine artery compression device includes left and right uterine artery compression devices disposed, respectively, on distal ends of the left and right rods,

guiding the rod includes guiding the left and right rods, via left and right first vaginal sites, such that distal ends of the rods are at left and right first extrauterine sites outside of but in a vicinity of left and right uterine arteries of the subject, respectively, and

compressing the uterine artery includes compressing the left and right uterine arteries, using, respectively, the left and right uterine artery compression devices.

For some applications, guiding the left and right rods includes guiding the rods to the left and right first extrauterine sites via vaginal tissue at approximately a six o'clock position with respect to the subject's uterus.

For some applications,

guiding the left rod includes guiding the left rod to the left first extrauterine site via vaginal tissue at approximately a three o'clock position with respect to the subject's uterus, and

guiding the right rod includes guiding the right rod to the right first extrauterine site via vaginal tissue at approximately a nine o'clock position with respect to the subject's uterus.

For some applications,

the method further includes inserting a rigid structure into the subject's body via the fornix-engaging structure, such that a distal end of the rigid structure passes through vaginal tissue at a second vaginal site, until the distal end of the rigid structure is at a second extrauterine site outside of the uterine artery,

and compressing the uterine artery using the uterine artery compression device includes compressing the uterine artery by squeezing tissue of the subject against the rigid structure, using the uterine artery compression device.

For some applications,

guiding the rod such that the distal end of the rod is at the first extrauterine site includes guiding the rod such that the distal end of the rod is at an extrauterine site that is posterior to a broad ligament of the subject, and

guiding the rigid structure such that the distal end of the rigid structure is at the second extrauterine site includes guiding the distal end of the rigid structure to a second extrauterine site that is anterior to a perivascular adipose layer of the broad ligament of the subject.

There is further provided, in accordance with some applications of the present invention, a method, including:

providing a rod having a telescoping distal portion thereof, a longitudinal axis thereof, and a balloon disposed around the telescoping portion;

inflating the balloon; and

while the balloon is in a subject's body, shortening the telescoping portion of the rod.

There is additionally provided, in accordance with some applications of the present invention, a method, including:

placing a measuring device in a vicinity of a plurality of blood vessels of a subject;

using the measuring device, measuring a parameter that is indicative of a level of blood-flow through the blood vessels; and

determining that blood flow through one of the blood vessels has stopped by detecting that the parameter has changed and then plateaued at a value indicating non-zero blood flow in the vicinity of the blood vessels.

There is further provided, in accordance with some applications of the present invention, a method for use with a subject's uterus, including:

inserting a distal end of an elongate element into the subject's uterus;

inflating a balloon disposed on the distal end of the elongate element, while the distal end of the elongate element is inside the subject's uterus; and

detecting a change in blood flow in a vicinity of the uterus by measuring a parameter in the vicinity using an oximeter that is disposed on the distal end of the elongate element, while the distal end of the elongate element is inside the subject's cervix.

For some applications, measuring the parameter in the vicinity comprises measuring a level of oxyhemoglobin in the vicinity.

For some applications, the parameter in the vicinity comprises measuring a pulse in the vicinity.

There is additionally provided, in accordance with some applications of the present invention, a method, including:

inserting a device inside a body of a subject, and outside of a uterus of the subject; and

anchoring the device by inserting a balloon into the subject's uterus and engaging the subject's uterus with the balloon by inflating the balloon, the balloon being coupled to the device.

There is further provided, in accordance with some applications of the present invention, a method for compressing a uterine artery of a subject, including:

placing a first compression device at an extrauterine site that is posterior to a broad ligament of the subject;

placing a second compression device at an extrauterine site that is anterior to a perivascular adipose layer of the broad ligament of the subject; and

compressing the uterine artery by squeezing tissue between the first and second compression devices.

For some applications, compressing the uterine artery includes avoiding occluding ureters of the subject.

There is additionally provided, in accordance with some applications of the present invention, a method for compressing a uterine artery of a subject, including:

providing a balloon having a first side and a second side, the first side being more flexible than the second side; and

compressing the uterine artery by:
placing the balloon such that the first side of the balloon faces the uterine artery; and
causing the balloon to expand at least in the direction of the uterine artery, by inflating the balloon.

There is further provided, in accordance with some applications of the present invention, a method, including:

providing left and right uterine artery compression devices coupled to each other by a flexible material; and

occluding left and right uterine arteries of a subject using, respectively, the left and right uterine artery compression devices.

For some applications, the flexible material defines a maximum distance between respective centers of the left and right uterine artery compression devices, and providing the left and right uterine artery compression devices includes providing the devices such that that the maximum distance is 5-25 cm.

For some applications, occluding includes inflating the uterine artery compression devices.

For some applications, occluding includes squeezing at least one of the uterine arteries between (a) apparatus placed into the subject and (b) one of the compression devices.

There is further provided, in accordance with some applications of the present invention, apparatus, including:

a device for restricting uterine blood flow of a subject, the device including:
at least one elongated support having a proximal end and a distal end, the at least one support being configured for disposition with the distal end at a location within a subject's abdomen and with the proximal end external to the subject;
at least one inflatable connected to the at least one support, the inflatable being configured to enter the subject's abdomen in a substantially non-inflated condition and to be inflated after entering the subject's abdomen, the inflatable being further configured and sized to exert a compression force on an exterior of a uterine artery of the subject; and
a surface configured to be disposed in opposition to the inflatable, the surface and the inflatable being configured to sandwich the uterine artery therebetween.

For some applications, the support is substantially rigid.

For some applications, the at least one inflatable includes a balloon.

For some applications, the at least one inflatable is further configured and sized to exert a compression force on an exterior of two uterine arteries of the subject.

For some applications, the at least one inflatable is configured to exert the compression force by exerting a compression force on the exterior of the uterine artery that is sufficient to at least partially occlude blood flow through the uterine artery.

For some applications, the device further includes a pressure sensor, and the at least one inflatable is configured to cooperate with the pressure sensor to determine a level of occlusion of the subject's uterine artery.

For some applications, the at least one support includes a first support and a second support and the at least one inflatable includes a first inflatable and a second inflatable, the first inflatable is connected to the first support and the second inflatable is connected to the second support, and the first inflatable and the second inflatable are configured to enter the subject's abdomen through an incision in a vaginal wall of the subject.

For some applications, the device further includes a flexible connecting member extending between and connecting the first and the second inflatables to one another.

For some applications, the flexible connecting member is configured to maintain the positions of the first and second inflatables relative to one another.

There is further provided, in accordance with some applications of the present invention, apparatus including:

a device for restricting uterine blood flow, the device including:
a first elongated pressure-applying member having a proximal end and a distal end, and being configured for disposition with the distal end at a location within an abdomen of the subject, and extending through the subject's vagina, with the proximal end external to the subject; and
a second elongated pressure-applying member having a proximal end and a distal end, and being configured for disposition with the distal end at a location within the subject's abdomen, and extending through the subject's vagina, with the proximal end external to the subject;
the first and second pressure-applying members being independently manipulatable from a location external to the subject.
the first and second pressure-applying members being configured to cooperate with one another to compress a uterine artery of the subject therebetween.

For some applications, the distal ends of the first and second pressure-applying members are configured to be inserted into the subject's abdomen via respective incisions in the subject's vagina.

For some applications, the first and second pressure-applying members are substantially rigid.

For some applications, the device further includes at least one inflatable disposed at the distal end of at least one of the first and second pressure-applying members.

For some applications, the device further includes:

a third elongated pressure-applying member having a proximal end and a distal end, and being configured for disposition with the distal end at a location within the subject's abdomen, and extending through the subject's vagina, with the proximal end external to the subject; and

a fourth elongated pressure-applying member having a proximal end and a distal end, and being configured for disposition with the distal end at a location within the subject's abdomen, and extending through the subject's vagina, with the proximal end external to the subject,

the third and fourth pressure-applying members being configured to cooperate with one another to compress a subject's uterine artery therebetween.

For some applications, at least two of the first, second, third, and fourth pressure-applying members are configured to be inserted into the subject's abdomen through a single incision in the subject's vagina.

For some applications, the third and fourth pressure-applying members are independently manipulatable from a location external to the subject.

There is further provided, in accordance with some applications of the present invention, apparatus including:

a device for restricting uterine blood flow, the device including:
a pressure surface configured for orientation within an abdomen of a subject;
a pressure-conveying member configured to extend between the pressure surface and a location outside of the abdomen; and
an intravaginal structure configured for orientation outside of the abdomen and configured to cooperate with the pressure-conveying member such that when the pressure-conveying member is seated against the intravaginal structure and removably coupled thereto, force is exerted from the pressure surface, through the pressure-conveying member, to the intravaginal structure,
the intravaginal structure being configured to cooperate with the pressure-conveying member in order to bias the pressure surface toward an external surface of a uterine artery of the subject so as to at least partially occlude the uterine artery.

For some applications, the pressure-conveying member is configured to lean on the intravaginal structure so as to urge the pressure surface into engagement with a body portion in the subject's abdomen, the body portion selected from the group consisting of tissue and a blood vessel.

For some applications, the intravaginal structure defines a passage configured to receive the pressure-conveying member therethrough and guide the pressure surface into engagement with a body portion in the subject's abdomen, the body portion selected from the group consisting of tissue and a blood vessel.

There is additionally provided, in accordance with some applications of the present invention, apparatus for use with a plurality of elongate rigid tools, including:

a surgical tool guide, including:
an intravaginal support structure having a central axis and shaped for insertion into a vagina of a subject; and
the support structure defining a plurality of openings that are shaped to direct respective ones of the elongate rigid tools from a location exterior to the vagina into a portion of an abdomen of the subject.

For some applications, the support structure is configured to engage a vaginal fornix of the subject.

For some applications, the support structure defines the plurality of openings, each of the openings extending at a non-zero angle with respect to the central axis.

For some applications, the support structure defines the plurality of openings, angles defined by each of the openings being configured to guide a respective one of the tools to a predetermined region of the abdomen.

For some applications, the support structure defines the plurality of openings, at least one of the plurality of openings being shaped as a slot configured to permit an angle of one of the tools that is inserted through the slot to be varied with respect to the central axis.

For some applications, the support structure defines the plurality of openings, a shape of at least one of the openings corresponding to at least one of the tools.

For some applications, at least one of the elongate rigid tools includes an elongate rigid tool that defines an inflatable at a distal end thereof, the inflatable being configured for inflation inside the subject's abdomen, and the intravaginal support structure is shaped to define at least one recess at a distal end of the intravaginal support structure, the recess being shaped to reduce squeezing of tissue of a cervix of the subject that is between the inflatable and the intravaginal support structure, upon the inflatable being inflated inside the subject's abdomen.

For some applications, the intravaginal support structure is shaped to define two recesses at the distal end of the intravaginal support structure, each recess being shaped to reduce squeezing of tissue of the cervix.

For some applications, the surgical tool guide includes an oximetry transmitter coupled to the intravaginal support structure.

For some applications, the surgical tool guide further includes an oximetry receiver coupled to the intravaginal support structure.

For some applications, the oximetry transmitter is configured to transmit light toward a cervix of the subject.

For some applications, the surgical tool guide further includes an oximetry receiver configured to receive light that is reflected from the cervix, the apparatus further including a control unit configured to determine a level of occlusion of the uterine artery in response to the received light.

For some applications, the support structure is shaped to define a central through-hole.

For some applications, the apparatus further includes an elongate anchoring element configured to secure the support structure in the subject's vagina, and the central through-hole is configured to receive the anchoring element.

For some applications, the anchoring element includes an expandable member on a distal end of the anchoring element, the expandable member being configured to enter the uterus through the cervix and to be expanded once inside the uterus, for exerting a holding force against an interior uterine wall.

There is further provided, in accordance with some applications of the present invention, apparatus including:

a device for measuring intrauterine oxygen levels, the device comprising:
an elongated arm; and
an oximeter that comprises an oximeter transmitter and an oximeter receiver, at least a portion of the oximeter being disposed on the elongated arm, and shaped for insertion into a uterus of a subject through a cervix of the subject, such that when the oximeter is within the subject's uterus, the elongated arm extends out of a vagina of the subject, and the oximeter is configured to relay to a location external to the uterus a signal that is indicative of a parameter of a portion of the subject's body selected from the group consisting of: the subject's uterus and the subject's cervix.

For some applications, the oximeter is configured to relay a signal that is indicative of a level of oxyhemoglobin at the selected portion.

For some applications, the oximeter is configured to relay a signal that is indicative of a pulse at the selected portion.

For some applications, the transmitter of the oximeter is disposed on the elongated arm.

For some applications, the receiver of the oximeter is disposed on the elongated arm.

For some applications, the transmitter and the receiver of the oximeter are disposed on the elongated arm.

For some applications, the apparatus further includes an intra-vaginal structure configured to be placed in the subject's vagina, and a portion of the oximeter is disposed on the intra-vaginal structure.

For some applications, the apparatus further includes a control unit configured to detect blood flow in a uterine artery of the subject, in response to the signal relayed by the oximeter.

For some applications, in response to the signal relayed by the oximeter, the control unit is configured to determine a level of occlusion of a uterine artery of the subject.

For some applications, the control unit is further configured to modulate occlusion of the uterine artery in response to the detected blood flow in the uterine artery.

For some applications, the control unit is configured to automatically modulate occlusion of the uterine artery in response to the detected blood flow in the uterine artery.

There is additionally provided in accordance with some applications of the present invention, apparatus, including:

an elongated arm having a distal end configured for location within a uterus of a subject;

an expandable configured for location on the elongated arm; and

a fornix cap configured for location within a vagina of the subject connected to the elongated arm, such that when the expandable is inserted through a cervix of the subject and expanded within the uterus, the expandable cooperates with the fornix cap through the elongated arm to hold the fornix cap against a fornix of the subject.

For some applications, the expandable includes a balloon.

For some applications, the fornix cap is shaped to define at least one tool guide, and the fornix cap, the elongated arm, and the expandable are configured such that the cooperation between the expandable and the fornix cap substantially fixes the tool guide in place with respect to an abdomen of the subject.

For some applications, the apparatus further includes a tool, and the fornix cap is configured such that the tool guide guides the tool from a location exterior to the vagina, to a predetermined region of the abdomen.

For some applications, the apparatus further comprises an oximeter configured to detect a parameter of a portion of the subject's body selected from the group consisting of: the subject's uterus and the subject's cervix, and at least a portion of the oximeter is disposed on the elongated arm.

There is further provided, in accordance with some applications of the present invention, apparatus including:

a device for measuring inter-uterine oxygen levels, the device including:
an intravaginal structure having a central axis and shaped for insertion into a vagina of a subject;
an elongated arm configured to be inserted into the subject's uterus via the central axis of the intravaginal structure; and
an oximeter, at least a portion of the oximeter being disposed on the elongated arm, and at least a portion of the oximeter being disposed on the intravaginal structure, the oximeter being configured to relay, to a location external to the vagina, a signal that is indicative of parameter of a portion of the subject's body selected from the group consisting of: the subject's uterus and a cervix of the subject.

For some applications, the oximeter is configured to relay a signal that is indicative of a level of oxyhemoglobin at the selected portion.

For some applications, the oximeter is configured to relay a signal that is indicative of a pulse at the selected portion.

There is additionally provided, in accordance with some applications of the present invention, apparatus, including:

an elongated arm having a distal end configured for location within a uterus of a subject and a proximal portion configured for location within a vagina of the subject;

a fornix cap configured for location within the subject's vagina and couplable to the elongated arm; and

an expandable coupled to the elongate arm for location within the subject's vagina, such that when the expandable is expanded within the vagina, the expandable cooperates with the fornix cap through the elongated arm to hold the fornix cap against a fornix of the subject.

The present invention will be more fully understood from the following detailed description of embodiments thereof, taken together with the drawings, in which:

### BRIEF DESCRIPTION OF THE DRAWINGS

FIG. 1 is a schematic illustration of a device configured to at least partially occlude a subject's uterine arteries, and a bed-side system for use with the device, in accordance with some applications of the present invention;

FIG. 2 is a schematic illustration of the device, in accordance with some applications of the present invention;

FIGS. 3A-F are schematic illustrations of the device of FIG. 2, the device including rigid structures that are placed anterior to the subject's uterine arteries, via a rod-guide of the device, in accordance with some applications of the present invention;

FIGS. 4A-B are schematic illustrations of a tube-guide, in accordance with some applications of the present invention;

FIG. 5 is a schematic illustration of the device configured to at least partially occlude a subject's uterine arteries, in accordance with some applications of the present invention;

FIGS. 6A-C are schematic illustrations of a tube-guide, in accordance with some applications of the present invention;

FIG. 7 is a schematic illustration of a positioning-anchoring balloon, and a positioning-anchoring tube that includes an oximetry system, in accordance with some applications of the present invention;

FIGS. 8A-C are schematic illustrations of a balloon for occluding the uterine artery, in accordance with respective applications of the present invention;

FIGS. 9, 10, 11, 12, 13A-B, and 14A-B are schematic illustrations of steps of a procedure for occluding a subject's uterine arteries, in accordance with some applications of the present invention;

FIGS. 15-16 are schematic illustrations of non-penetrating rigid structures, in accordance with some applications of the present application;

FIGS. 17A-B are schematic illustrations of a locking mechanism for locking penetrating and non-penetrating rigid structures, in unlocked (17A) and locked (17B) states, in accordance with some applications of the present invention;

FIGS. 18A-G are schematic illustrations of non-penetrating rigid structures, in accordance with respective applications of the present invention;

FIGS. 19-23, and 24A-B are schematic illustrations of steps of a procedure for occluding a subject's uterine arteries using a two-portion fornix-engaging structure, in accordance with some applications of the present invention;

FIG. 25 is a schematic illustration of a three-portion fornix-engaging structure, in accordance with some applications of the present invention;

FIGS. 26-31, 32A-B, and 33-39 are schematic illustrations of steps of a procedure that is used with the three-portion fornix-engaging structure, in accordance with some applications of the present invention;

FIGS. 40A-H are schematic illustrations of decouplable rigid-structure-support-elements, in accordance with some applications of the present invention;

FIGS. 41A-H are schematic illustrations of left and right balloons that are used as a uterine artery compression device, in accordance with some applications of the present invention;

FIGS. 42A-E are schematic illustrations of a first portion of the three-portion fornix-engaging structure, in accordance with some applications of the present invention;

FIGS. 43A-C are schematic illustrations of the three-portion fornix-engaging structure, in accordance with some applications of the present invention;

FIGS. 44A-B are schematic illustrations of apparatus for occluding uterine arteries, including one or more double-chambered balloons, in accordance with some applications of the present invention;

FIGS. 45A-D are schematic illustrations of a double-chambered balloon, in accordance with some applications of the present invention;

FIGS. 46A-C are schematic illustrations of a double-chambered balloon, in accordance with some applications of the present invention; and

FIGS. 47A-B are schematic illustrations of left and right single-chambered balloons, in accordance with some applications of the present invention.

### DETAILED DESCRIPTION OF EMBODIMENTS

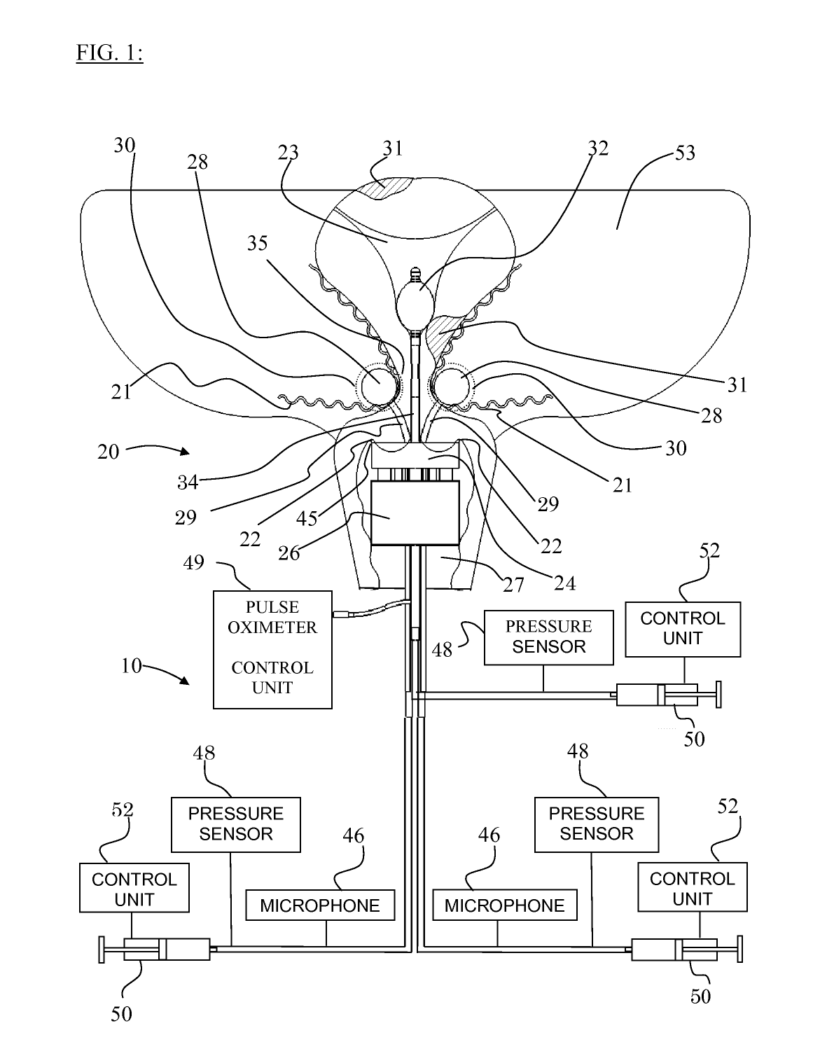

Reference is now made to FIG. 1, which is a schematic illustration of a device 20 configured to at least partially occlude a subject's uterine arteries 21, in accordance with some applications of the present invention. The occlusion of the uterine arteries is typically performed in order to reduce the supply of blood to uterine fibroids 31, thereby causing irreversible ischemic necrosis and death of the fibroids.

Typically, a fornix-engaging structure 24 (i.e., a fornix cap) is inserted into the subject's vagina 27, such that it engages the vaginal fornix 45. Subsequently, an incision 22 is made in the vaginal wall. (Although the incision in the vaginal wall is shown at 3 o'clock and 9 o'clock positions, for some applications, incision 22 is made at other positions on the vaginal wall, for example, at a 6 o'clock, a 2 o'clock, and/or a 10 o'clock position, as described in further detail hereinbelow.) A tube-guide 26 (which is described in further detail hereinbelow) is coupled to fornix-engaging structure 24. A uterine artery compression device, for example, an inflatable and/or an expandable element, such as a balloon 28 (as shown), is disposed on the distal end of a tube 29. (For some applications, as described hereinabove, a solid rod is used in place of tube 29 (i.e., a hollow rod), mutatis mutandis). The tube is inserted into the subject's body, and the tube-guide guides the tube such that the distal end of the tube passes through vaginal tissue until the distal end of the tube is at a site 30 that is adjacent to the subject's cervix 35, e.g., above the subject's fornix 45, and below the subject's uterus 23. Site 30 is typically outside of uterine artery 21, but in a vicinity of a portion of the uterine artery that supplies uterine fibroid 31. Site 30 is typically posterior to the subject's broad ligament 53. The tube is typically inserted via incision 22. While the distal end of the tube is at site 30, the uterine compression device is used to compress, and at least partially occlude the subject's uterine artery. For example, while the distal end of the tube is at site 30, the balloon is inflated in order to occlude the uterine artery.

(The terms “guide structure,” “tube-guide,” and “rod guide” are used in the present application. A “tube guide” is a type of “rod guide,” and a “rod guide” is a type of “guide structure.” The scope of the present invention includes using any rod guide (e.g., a solid rod guide) in place of a tube guide, for applications in which the use of a tube guide is described, mutatis mutandis. The scope of the present invention further includes using a different guide structure in place of a tube guide or a rod guide, for applications in which the use of a tube guide or a rod guide is described, mutatis mutandis.)

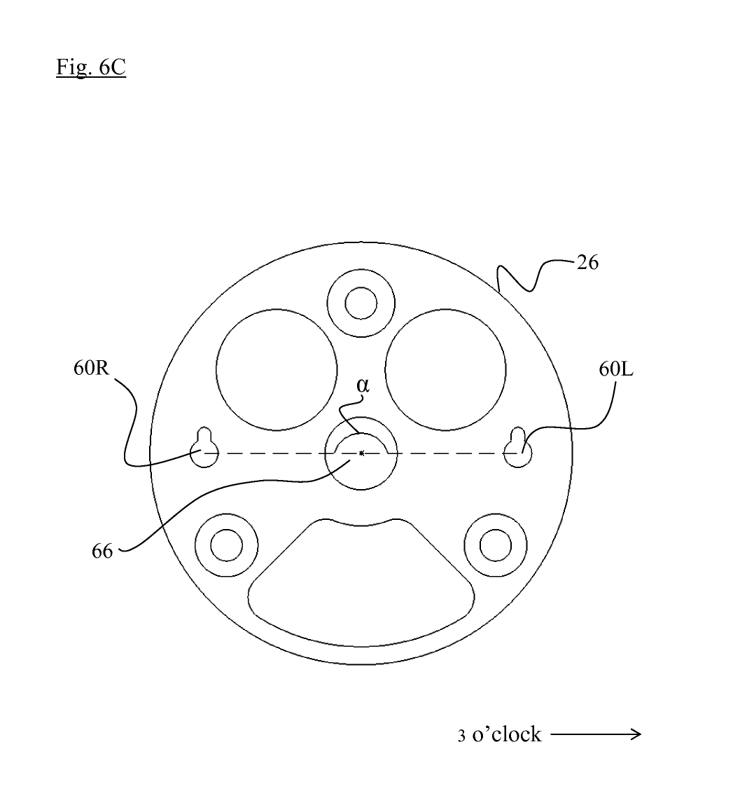

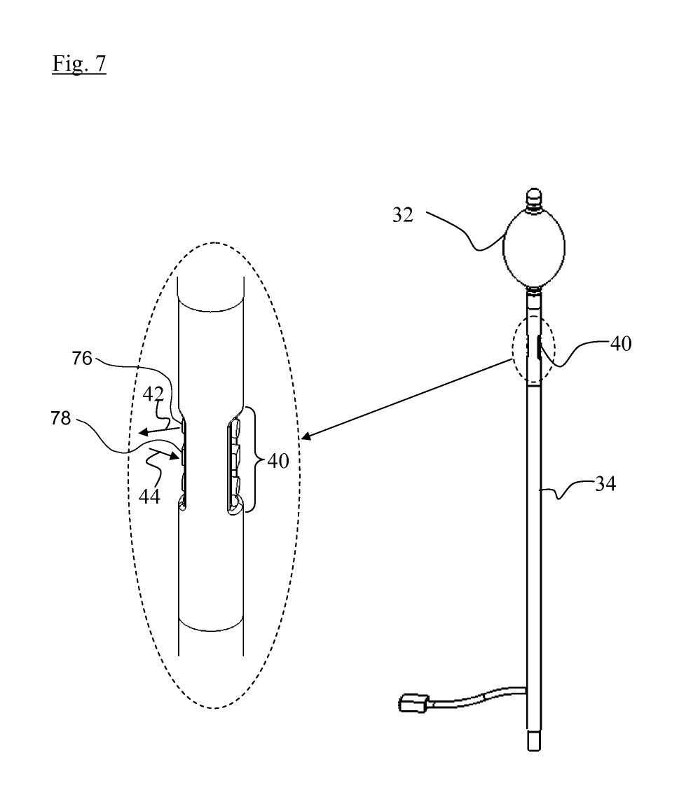

A bed-side system 10 of device 20 typically includes control units 52 for controlling inflation of balloons of device 20, and a monitor (not shown) that displays readings of sensors (e.g., pressure sensors 48, microphones 46, and/or oximeters 40 (shown in FIG. 7)) that sense parameters associated with the device, as described in further detail hereinbelow.

Typically, the distal end of tube 29 is blunt, for applications in which tube 29 is inserted via incisions that have previously been made in the vaginal tissue, as described hereinabove. For some applications, the distal end of tube 29 is sharp, and is used to penetrate the vaginal tissue by forming incisions in the vaginal tissue. For some applications, in order to prevent the sharp distal end of tube 29 from damaging tissue in the vicinity of the tube, the sharp distal end is folded subsequent to the penetration of the vaginal tissue by the distal end of the tube. For some applications, techniques that are known in the art (such as those that are used with laparoscopic tools) are used for folding the sharp distal end of the tube.

For some applications, tube-guide 26 and fornix-engaging structure 24 are coupled to each other, such that they form a single integrated unit at the time of manufacture, i.e., the fornix-engaging structure includes a guiding structure for guiding tube 29, and/or other portions of device 20. (For example, the fornix-engaging structure may define one or more holes that are configured to guide portions of device 20.) Alternatively, the tube-guide and the fornix-engaging structure are reversibly couplable to each other (e.g., as shown in FIG. 4A). For some applications, tube 29 is inserted into the subject's body, via the fornix-engaging structure, prior to the tube-guide being coupled to the fornix-engaging structure. Subsequently, the tube-guide is coupled to the fornix-engaging structure, thereby guiding the distal end of the tube-guide to site 30. Typically, for such applications, prior to the tube-guide being coupled to the fornix-engaging structure, tube 29 is able to rotate with respect to the fornix-engaging structure. For example, as seen in FIG. 4A, the size of hole 35 in the fornix-engaging structure (through which the tube is inserted) is substantially larger than the cross-section of the tube. After the tube-guide has subsequently been coupled to the fornix-engaging structure, the tube is inhibited by the tube-guide from rotating with respect to the fornix-engaging structure. Typically, the shape and size of hole 60 (shown in FIG. 4A) in the tube-guide through which the tube is inserted is substantially similar to the cross-section of the tube.

For some applications (as shown), left and right balloons are inserted into sites 30 on, respectively, the left and right side of the subject's cervix. The balloons are used to compress, and at least partially occlude, left and right uterine arteries of the subject, in accordance with the techniques described herein. Typically, the left and right arteries are compressed simultaneously. Alternatively the left and right arteries are compressed at separate times. For some applications, apparatus and methods that are described herein, as being applied to a single uterine artery are applied to left and right uterine arteries, at least partially, simultaneously.

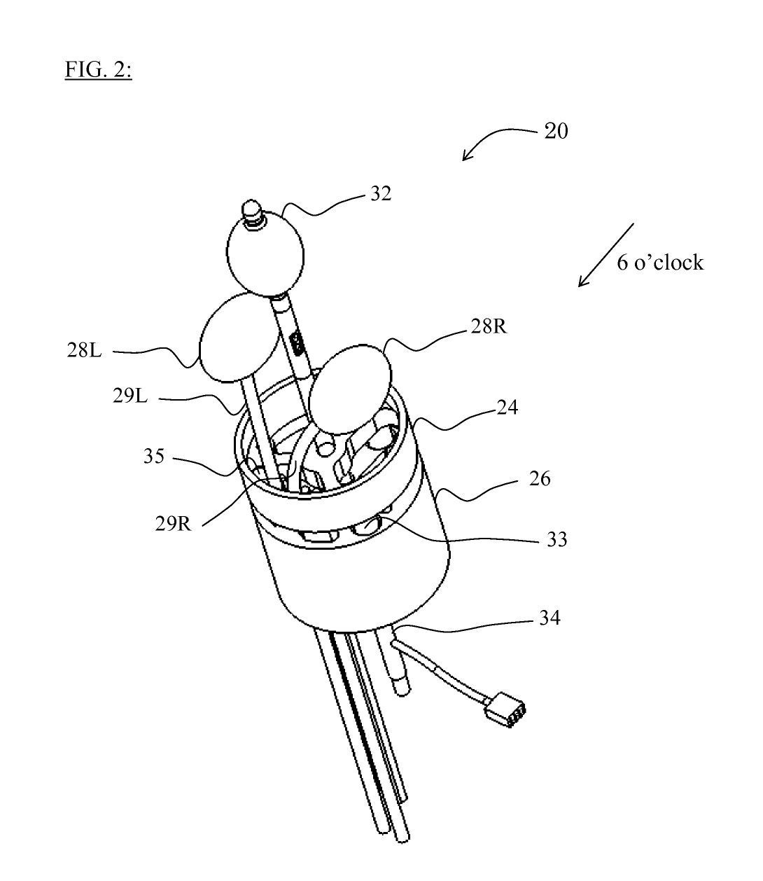

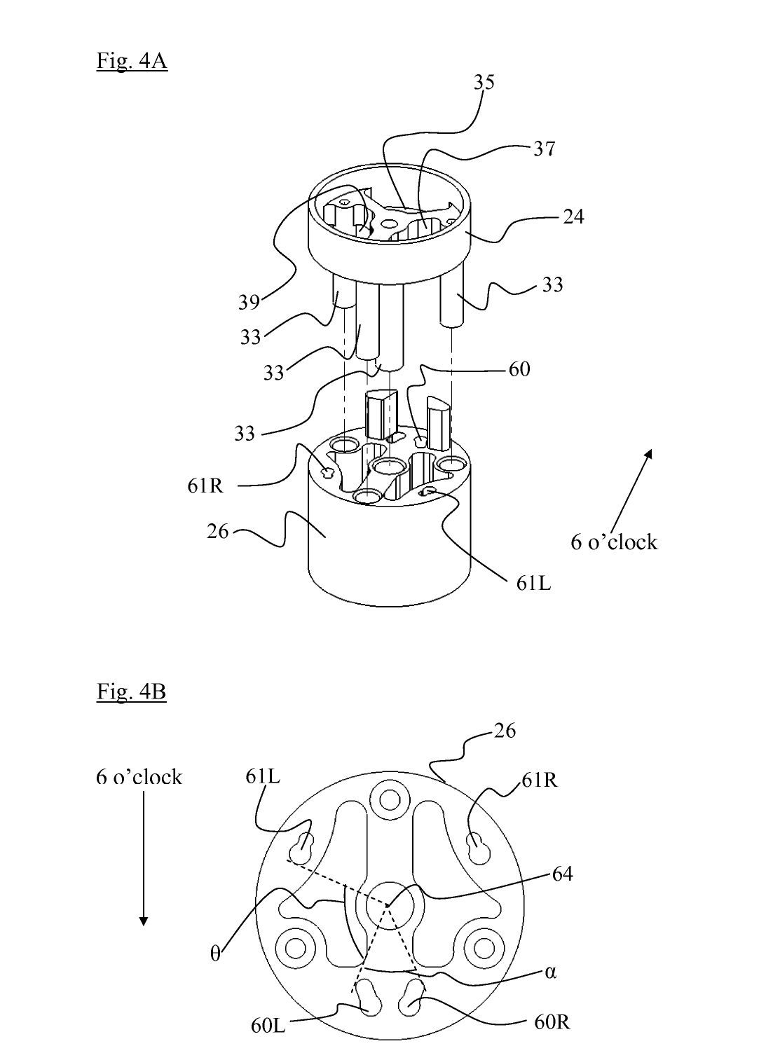

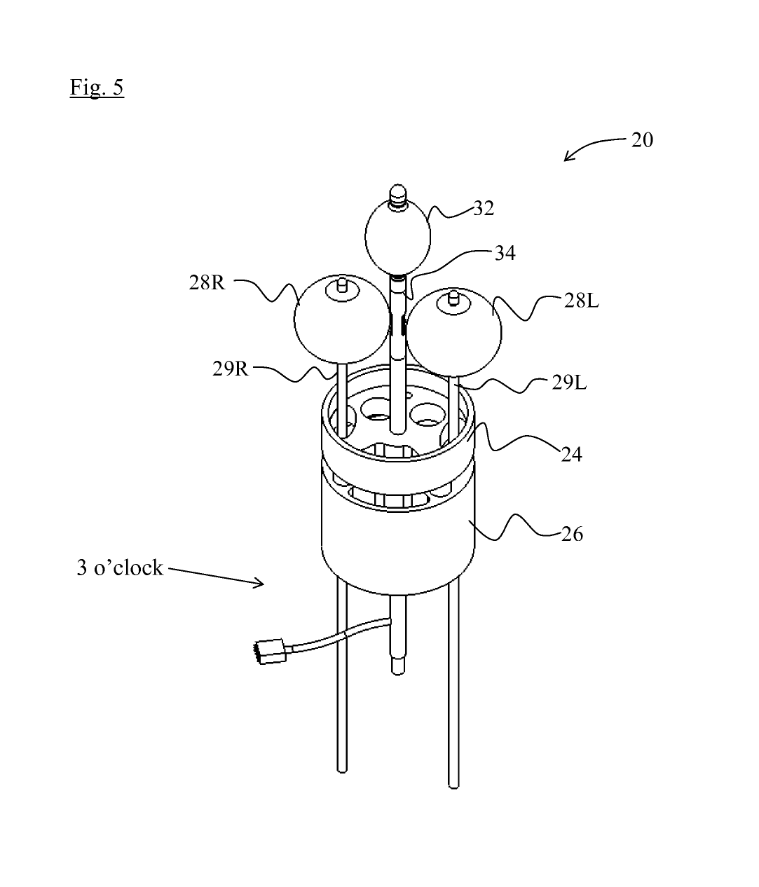

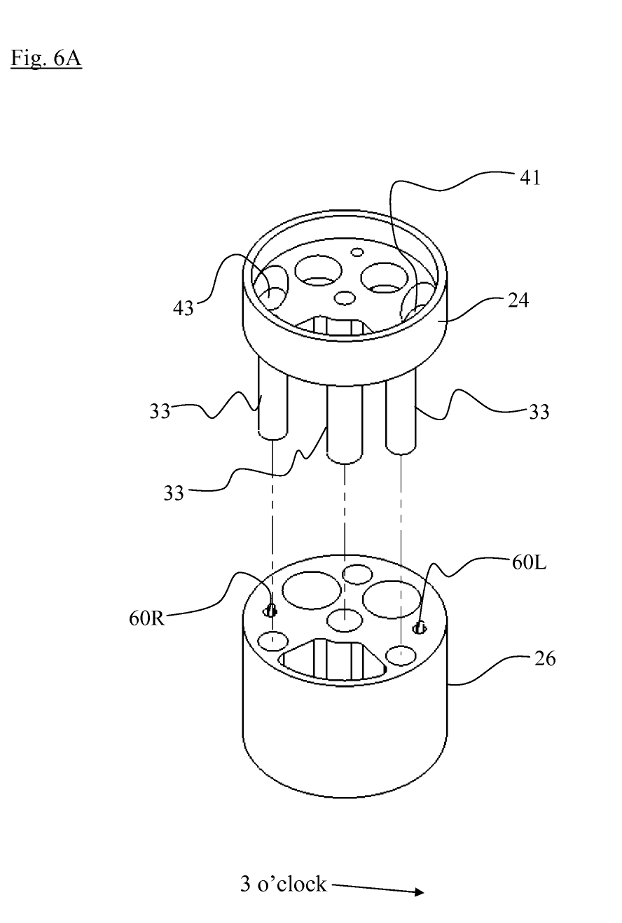

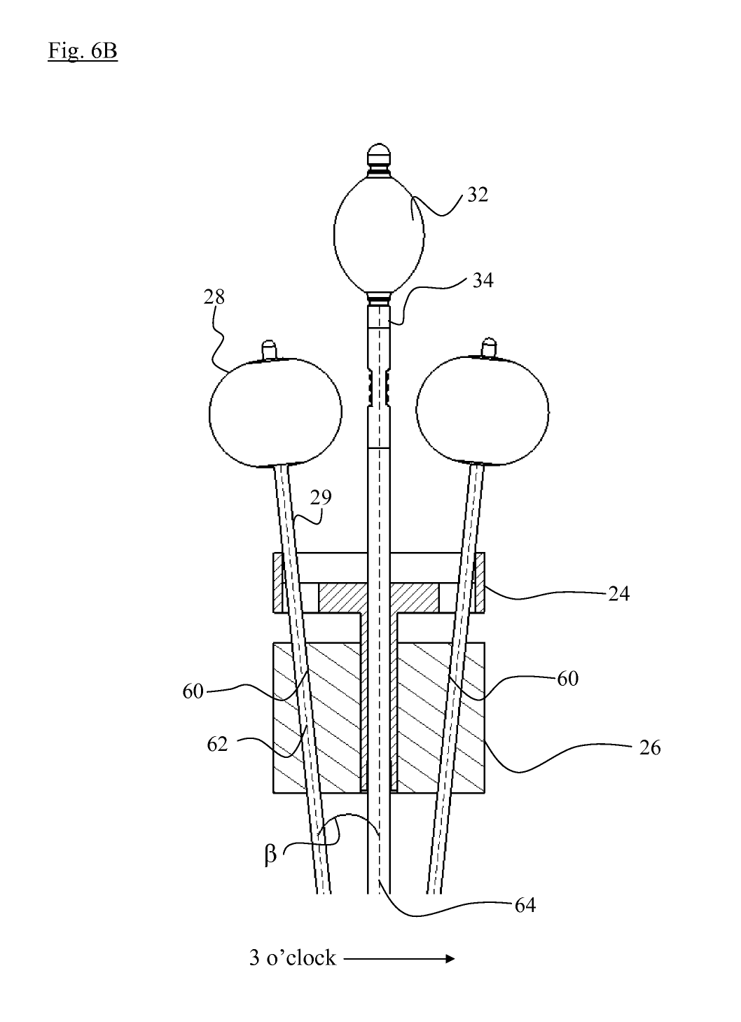

For some applications, a single (posterior) incision is made at the 6 o'clock position of the fornix (i.e., a posterior colpotomy is performed at the posterior fornix), and left and right balloons 28 are inserted via the single incision. Device 20, as used for such applications, is shown in FIGS. 2-4. Alternatively, left and right balloons 28 are inserted into sites 30 on the left and right side of the subject's cervix via incisions 22 at approximately 3 o'clock and 9 o'clock positions of the fornix (i.e., the lateral fornix) with respect to the subject's uterus. Device 20, as used for such applications, is shown in FIGS. 5 and 6A-C.

(It is noted that in the context of the present application, the term “colpotomy” refers to an incision in a subject's vaginal tissue. The terms “anterior colpotomy” and “posterior colpotomy” refer respectively to incisions in the vaginal tissue at approximately 12 o'clock and 6 o'clock positions.)

For some applications, a positioning-anchoring balloon 32 (i.e., an expandable), disposed at the distal end of a positioning-anchoring tube 34 (i.e., an elongated arm) is inserted into the subject's uterus, via the subject's cervix. (For some applications, a solid rod is used in place of tube 34 (which is a hollow rod), mutatis mutandis.) The intrauterine positioning-anchoring balloon is inflated while it is disposed in the uterus, in order to position and then anchor device 20. For example, balloon 32 may be used to position and anchor extrauterine compression balloons 28 with respect to the subject's cervix and uterine arteries. For some applications, a balloon that is similar to the silicone balloon of The Rumi System® is used as the intrauterine balloon. For some applications, the length of the positioning-anchoring tube with respect to the fornix-engaging structure is adjustable. For example, the positioning-anchoring tube may be threadedly coupled to the fornix-engaging structure and/or the tube-guide, such that the height of the positioning-anchoring tube with respect to the fornix-engaging structure is controlled by screwing the positioning-anchoring tube through the fornix-engaging structure and/or the tube-guide.

(Included within the scope of the present invention is a positioning-anchoring balloon that is inserted into a subject's uterus and which, by engaging the subject's uterus by being inflated, positions, and then anchors a medical tool that is in an extrauterine location within the subject's body.)

For some applications, uterine artery 21 is compressed due to each of left and right balloons 28 squeezing (i.e., forcing) tissue toward the other balloon. Alternatively or additionally, one or both of balloons 28 squeeze tissue against positioning-anchoring tube 34, and/or positioning-anchoring balloon 32. For some applications, one or both of balloons 28 squeeze tissue against fornix-engaging structure 24. For some applications, balloon 28 compresses the uterine artery by generally causing compression of tissue in the vicinity of the balloon, and without squeezing the subject's tissue against another portion of device 20.

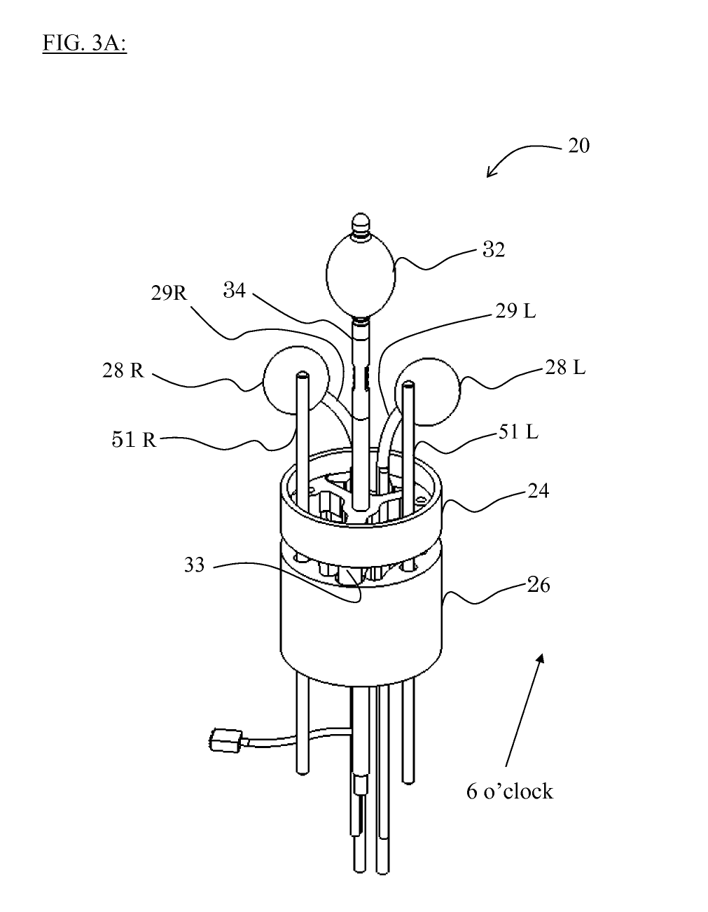

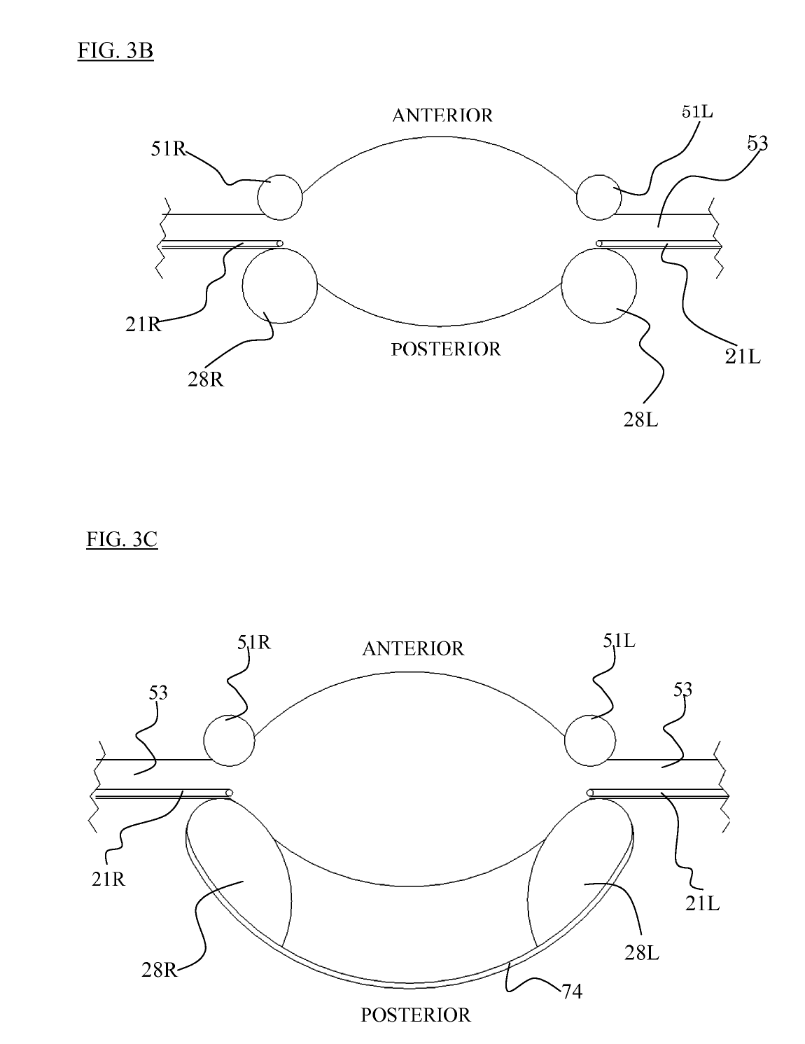

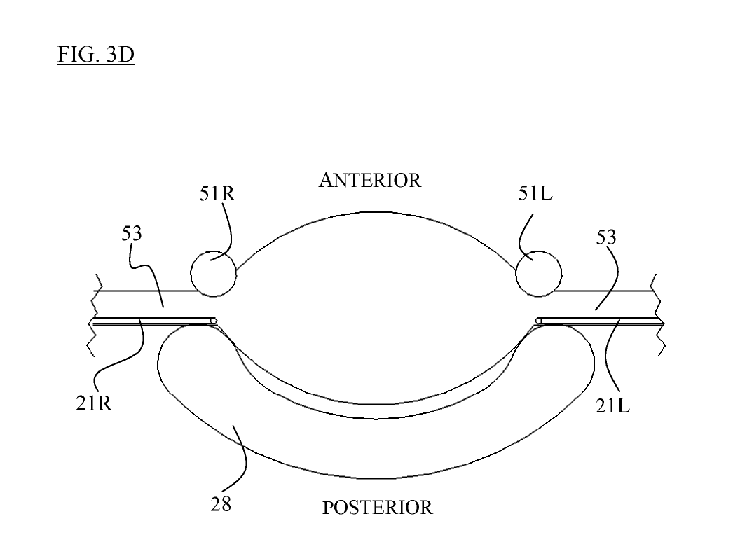

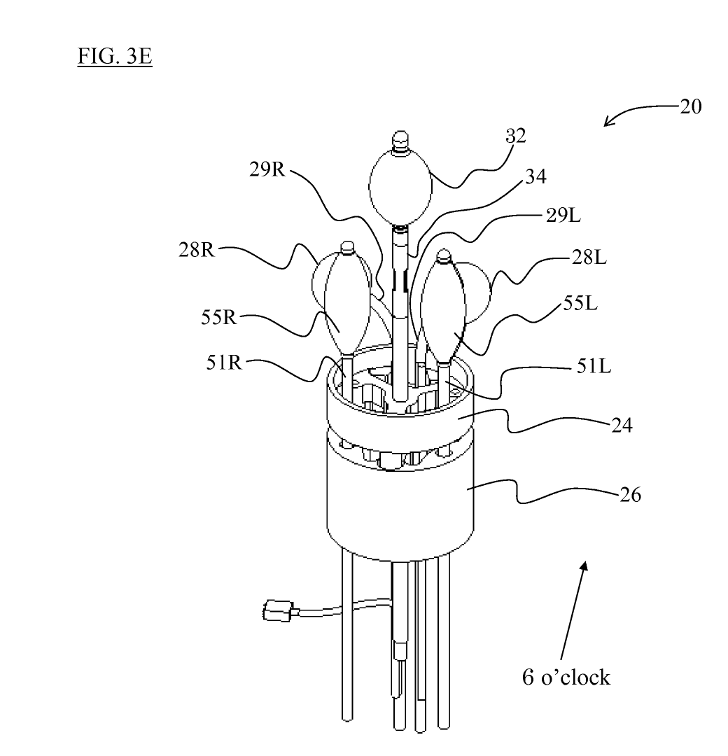

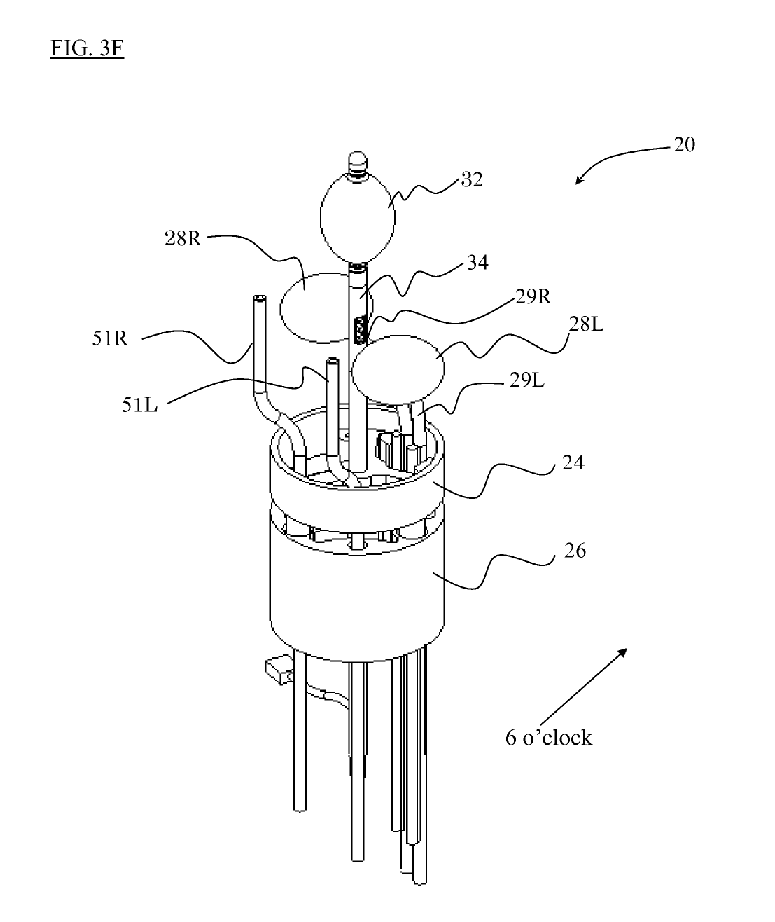

For some applications, first and second rigid structures 51 are inserted into left and right extrauterine positions anterior to the left and right broad ligaments (as shown in FIG. 3B). For some applications, rigid structures 51 are uterine artery compression devices. The left and right uterine artery compression devices 28 are inserted into left and right extrauterine positions posterior to the left and right broad ligaments. The left and right uterine artery compression devices 28 compress the left and right uterine arteries by squeezing tissue against, respectively, the first and second rigid structures. For some applications, a single uterine artery compression device is placed at a site that is posterior to the broad ligament and the single uterine artery compression device squeezes both the left and right uterine arteries, respectively, against the left and right rigid structures (as shown in FIG. 3C).

Typically, tube 29 is a rigid tube. The rigidity of the tube maintains the position of the distal end of the tube at site 30, while balloon 28 is inflated and compresses the uterine artery. (If tube 29 were not rigid, then some of the inflation of the balloon would be in effect wasted. In some cases, this might cause the tube to bend away from the uterine artery, such that the balloon does not have sufficient compressive effect to occlude the uterine arteries.) For some applications, tube 29 is flexible, for example, during insertion of the tube through the vagina, but becomes rigid for compressing the uterine artery. Typically, fornix-engaging structure 24, tube-guide 26, and positioning-anchoring tube 34 are also rigid.

Reference is now made to FIG. 2, which is a schematic illustration of device 20, in accordance with some applications of the present invention. For some applications, a single incision is made in the vaginal tissue at approximately the 6 o'clock position, and left and right tubes 29 and balloons 28 are inserted via the single incision. Tube-guide 26 defines two holes 60 (shown in FIG. 4A) at approximately the 6 o'clock position. Tubes 29L and 29R are both inserted through the incision in the vaginal tissue at the 6 o'clock position by fornix-engaging structure 24 being pushed against the subject's fornix, and the tubes being inserted through a hole 35 in structure 24.

For some applications, subsequent to the fornix-engaging structure being pushed against the fornix, and/or subsequent to tubes 29 having been inserted through hole 35 in structure 24, tube-guide 26 is coupled to structure 24 via coupling elements 33. The tube-guide guides left and right tubes 29L and 29R to their respective positions, by being coupled to the fornix-engaging structure. As shown in FIG. 3B, the distal ends of tubes 29L and 29R are appropriately shaped for balloons 28L and 28R to be positioned adjacent to left and right uterine arteries 21L and 21R posterior to broad ligaments 53 by being inserted through the incision at the 6 o'clock position.

Reference is now made to FIGS. 3A-F, which are schematic illustrations of device 20 including rigid structures 51, in accordance with some applications of the invention. For some applications, rigid structures 51 are solid rods, or hollow rods (i.e., tubes). For some applications, in addition to an incision being made in the vaginal tissue at approximately the 6 o'clock position (as described with reference to FIG. 2), incisions are made at approximately the 2 o'clock and 10 o'clock positions of the vaginal fornix. Left rigid structure 51L is inserted to an extrauterine site that is anterior to the broad ligament in the vicinity of left uterine artery 21L (shown in FIG. 3B), via the 2 o'clock incision. Alternatively or additionally, right rigid structure 51R is inserted to an extrauterine site that is anterior to the broad ligament in the vicinity of right uterine artery 21R (also shown in FIG. 3B), via the 10 o'clock incision. Left and right extrauterine sites of the left and right rigid structures are typically anterior to, respectively, the subject's left and right broad ligaments 53. Typically, as with tube 29, left and right rigid structures 51L and 51R are inserted into the subject's body via fornix-engaging structure 24. Subsequently, tube-guide 26 is coupled to the fornix-engaging structure and guides the rigid structures to their respective sites.

For some applications, uterine artery compression device 28 includes left and right uterine artery compression devices 28L and 28R. The left uterine artery compression device compresses left uterine artery 21L, by squeezing the subject's broad ligament and the uterine artery against left rigid structure 51L, as shown in FIG. 3B. The right uterine artery compression device compresses right uterine artery 21R, by squeezing the subject's broad ligament and the uterine artery against right rigid structure 51R, also as shown in FIG. 3B. The compression of the uterine arteries is thus performed in an anterior-posterior direction, and not in a lateral direction. For some applications, by performing the uterine artery compression in the anterior-posterior direction, occlusion of the subject's ureters is reduced or avoided. Alternatively, for some applications, the subject's uterine arteries are compressed in a lateral direction. Typically, using rigid structures 51L and 51R in addition to the uterine artery compression device facilitates compression of the uterine arteries, as the rigid structures provide resistance against which to push the uterine arteries. Typically, inserting the rigid structures at the 2 o'clock and 10 o'clock positions ensures that the rigid structures are inserted to sites that are anterior to the broad ligaments.

For some applications, left and right uterine artery compression devices 28 are balloons that are coupled to at least one flexible connecting member 74, as shown in FIG. 3C, and as described in detail hereinbelow. For some applications, uterine artery compression device 28 is a single uterine artery compression device, for example, a curved balloon, as shown in FIG. 3D. The uterine artery compression device compresses left and right uterine arteries by squeezing the broad ligament and the uterine arteries against, respectively, left and right rigid structure 51L and 51R.

For some applications, balloons 55L and 55R are disposed on the distal ends of, respectively, rigid structure 51L and rigid structure 51R, as shown in FIG. 3E. Typically the balloons facilitate compression of the uterine arteries, and/or facilitate measurement of the pressure in the vicinity of the uterine arteries, as described in further detail hereinbelow. For some applications, balloons 55 are uterine artery compression devices. For some applications, distal portions of rigid structures 51L and 51R are curved, as shown in FIG. 3F, to facilitate insertion of the structures to suitable extrauterine sites. Alternatively, the distal portions of the rigid structures are substantially straight, as shown in FIG. 3A. For some applications, left rigid structure 51L and left tube 29L are pivotally connected to each other, e.g., structure 51L and tube 29L may comprise a clamp (pivot not shown). Similarly, for some applications, right rigid structure 51R and right tube 29R are pivotally connected to each other. For example, left rigid structure 51L and left tube 29L, and/or right rigid structure 51R and right tube 29R, act as a hemostat.

It is noted that left rigid structure 51L and left tube 29L (and, similarly, right rigid structure 51R and right tube 29R) may be viewed as, respectively, first and second elongated pressure-applying members having proximal and distal ends, and being configured for disposition with the distal end at a location within the subject's abdomen, and extending through the subject's vagina, with the proximal end external to the subject. The first and second pressure-applying members are independently manipulatable from a location external to the subject, and are configured to cooperate with one another to compress the subject's uterine artery therebetween.

Reference is now made to FIGS. 3A-F and to FIG. 4A, FIG. 4A being a schematic illustration of fornix-engaging structure 24 and tube-guide 26 in a decoupled state, in accordance with an application of the present invention. For some applications, device 20 is placed inside the subject's body in accordance with the following procedure:

(1) Positioning-anchoring tube 34 and positioning-anchoring balloon 32 are coupled to fornix-engaging structure 24.

(2) Before or after step (1), fornix-engaging structure 24 is inserted into the subject's vagina such that it engages the subject's fornix, and such that the distal end of positioning-anchoring tube 34 and the balloon 32 are placed inside the subject's uterus, via the subject's cervix.

(3) Positioning-anchoring balloon 32 is inflated so as to position and anchor the fornix-engaging structure.

(4) Incisions are made in the fornix (a) at the 6 o'clock position, via hole 35, (b) at the 2 o'clock position, via a hole 37, and (c) at the 10 o'clock position, via a hole 39.

(5) Left and right tubes 29 are inserted into the extrauterine space inside the subject's body, via the 6 o'clock incision in the fornix.

(6) Left and right rigid structures 51 are inserted, respectively, via the 2 o'clock and 10 o'clock incisions in the fornix.

(7) Tube-guide 26 is coupled to the fornix-engaging structure, via coupling elements 33. During the coupling of the tube-guide to the fornix-engaging structure, tubes 29 are inserted into holes 60 of the tube-guide, and rigid structures 51 are inserted into holes 61 of the tube-guide. Thus, by being coupled to the fornix-engaging structure, the tube-guide guides the tubes and the rigid structures to their respective intra-procedural sites.

(8) Balloons 28 on the distal ends of tubes 29 are inflated such that the left and right uterine arteries are compressed, by the broad ligaments and uterine arteries being compressed against the rigid structures.

Reference is now made to FIG. 4B, which is a schematic top-view illustration of tube-guide 26, in accordance with some applications of the invention. For some applications, an angle theta, defined by hole 61L (through which rigid structure 51L is guided), longitudinal axis 64 of the tube-guide, and hole 60L (through which tube 29L is guided), is greater than 100 degrees. Similarly, the angle defined by hole 61R (through which rigid structure 51R is guided), longitudinal axis 64 of the tube-guide, and hole 60R (through which tube 29R is guided), is greater than 100 degrees. For some applications, an angle alpha defined by left hole 60L, longitudinal axis 64 of the tube-guide, and right hole 60R, is less than 10 degrees.

Reference is now made to FIG. 5, which is a schematic illustration of device 20, in accordance with some applications of the present invention. As stated hereinabove, for some applications, left and right balloons 28L and 28R are inserted into sites on both the left and right side of the subject's cervix via incisions 22 at approximately 3 o'clock and 9 o'clock positions with respect to the subject's uterus. Device 20, as used for such applications, is shown in FIG. 5. Fornix-engaging structure 24 and tube-guide 26 each define holes at the 3 o'clock and 9 o'clock positions. Tubes 29L and 29R are inserted through incisions in the vaginal tissue at the 3 o'clock and 9 o'clock positions by fornix-engaging structure 24 being pushed against the subject's fornix, and the tubes being inserted through the holes in the structure. For some applications, subsequent to the fornix-engaging structure 24 being pushed against the fornix, and/or subsequent to the tubes having been inserted through the holes in structure 24, tube-guide 26 is coupled to structure 24, via coupling elements 33. The tube-guide guides left and right tubes 29L and 29R to their respective positions, by being coupled to the fornix-engaging structure.

Reference is now made to FIGS. 6A-C, which are schematic illustrations of components of device 20, in accordance with some applications of the present invention. The fornix-engaging structure 24 and tube-guide 26, shown in FIGS. 6A-C, are typically used in techniques in which incisions 22 are made in the vaginal tissue at approximately 3 o'clock and 9 o'clock positions with respect to the subject's uterus (i.e., as shown in FIG. 5). However, some of the elements of device 20 described with reference to FIGS. 6A-C are also used with the fornix-engaging structure and tube-guide as described elsewhere in this application.

For some applications, device 20 is placed inside the subject's body in accordance with the following procedure:

(1) Positioning-anchoring tube 34 and positioning-anchoring balloon 32 are coupled to fornix-engaging structure 24.

(2) Before or after step (1), fornix-engaging structure 24, is inserted into the subject's vagina such that it engages the subject's fornix, and such that the distal end of positioning-anchoring tube 34 and balloon 32 are placed inside the subject's uterus, via the subject's cervix.

(3) Positioning-anchoring balloon 32 is inflated so as to position and anchor the fornix-engaging structure.

(4) Incisions are made in the fornix (a) at the 3 o'clock position, via a hole 41 in structure 24, and (b) at the 9 o'clock position, via a hole 43 in structure 24.

(5) Left and right tubes 29 are inserted into the extrauterine space inside the subject's body, via, respectively, the 3 o'clock and 9 o'clock incisions in the fornix.

(6) Tube-guide 26 is coupled to the fornix-engaging structure, via coupling elements 33. During the coupling of the tube-guide to the fornix-engaging structure, tubes 29 are inserted into holes 60 of the tube-guide. Thus, by being coupled to the fornix-engaging structure, the tube-guide guides the tubes to their respective intra-procedural sites.

(8) Balloons 28 on the distal ends of tubes 29 are inflated such that the left and right uterine arteries are compressed, typically, by tissue being compressed against a portion of device 20, e.g., positioning-anchoring tube 34.

For some applications, as shown in FIG. 6B, longitudinal axis 62 of hole 60 is not parallel to longitudinal axis 64 of the fornix-engaging structure. For example, axis 62 may be at an angle beta of even as large as 60 degrees, although beta is typically less than 60 degrees, e.g., 10 degrees to 45 degrees from axis 64, for example, 15 degrees to 30 degrees from axis 64. For some applications, due to the orientation of hole 60, tube 29 is inserted into the subject's body at an angle to axis 64. For some applications, this ensures that the distal end of tube 29 becomes positioned in a suitable position for balloon 28 to occlude the subject's uterine artery.

For some applications, the length of tube 29 that protrudes from structure 24 into the subject's body is between 1 cm and 6 cm. For some applications, tube 29 includes a stopper (not shown) at its distal end, in order to prevent the tube from being inserted too far through the tube-guide. For example, a stopper may be used as described in PCT

Publication WO 08/012,802 to Gross, which is incorporated herein by reference, mutatis mutandis.

For some applications, tube-guide 26 defines a further hole 66 (shown in FIG. 6C), through which positioning-anchoring tube 34 is inserted into the subject's uterus. For some applications, positioning-anchoring tube 34 includes a stop at its distal end in order to prevent the positioning-anchoring tube from being inserted too far through the tube-guide.

For some applications, as shown in FIG. 6C, hole 60 is not circular. (Alternatively, hole 60 is circular.) For some applications, tube 29 is shaped such that the cross-section of tube 29 has the same shape as that of hole 60. Further typically, tube 29 and hole 60 are shaped so as to prevent tube 29 from rotating with respect to tube-guide 26.

For some applications, angle alpha defined by left hole 60L in tube-guide 26, longitudinal axis 64 of the tube-guide, and right hole 60R in tube-guide 26, is between 170 and 190 degrees.

Reference is now made to FIG. 1, and to FIG. 7, FIG. 7 being an enlarged schematic illustration of positioning-anchoring tube 34 and positioning-anchoring balloon 32, in accordance with some applications of the present invention. For some applications, device 20 includes one or more sensors for monitoring blood flow, in order to evaluate the occlusion of the uterine artery. For example, a microphone 46 detects sound waves that are generated from the uterine artery at site 30 (shown in FIG. 1), and/or at balloon 55 (application not shown), and/or a pressure sensor 48 detects the pressure inside balloon 28 (shown in FIG. 1), balloon 32 (shown in FIG. 1), and/or balloons 55 (application not shown).

For some applications, one or more of the sensors are coupled to positioning-anchoring tube 34. For example, device 20 may include one or more oximeters 40, which are coupled to positioning-anchoring tube 34, as shown. The oximeters emit light (e.g., red and infrared light) toward the cervix, or toward tissue in the vicinity of the cervix, and detect the light that is reflected from the cervix, or from the tissue. For example, light is emitted from an oximeter transmitter 76 in the direction of arrows 42, and light that is reflected in the direction of arrows 44 is detected by an oximeter receiver 78. For some applications, the emitted and reflected beams of light are parallel to each other. Alternatively, the light is emitted in generally all directions, and a portion of the reflected light is detected. A pulse oximeter control unit 49 (shown in FIG. 1) is typically configured to detect capillary flow in the uterine arteries, the uterus, and/or the cervix by the oximeter detecting a level of oxyhemoglobin and/or deoxyhemoglobin in the uterine arteries, the uterus, and/or the cervix, and/or by detecting a pulse caused by blood flow in one or more of the aforementioned locations. In response thereto, the pulse oximeter control unit typically generates an output that is displayed on a monitor (not shown) of bed-side system 10 (shown in FIG. 1).

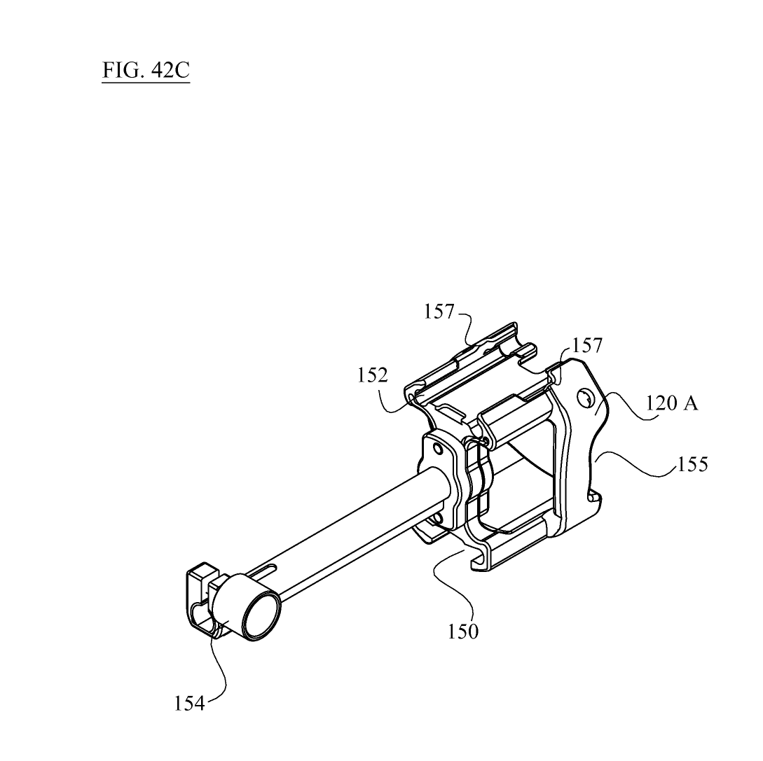

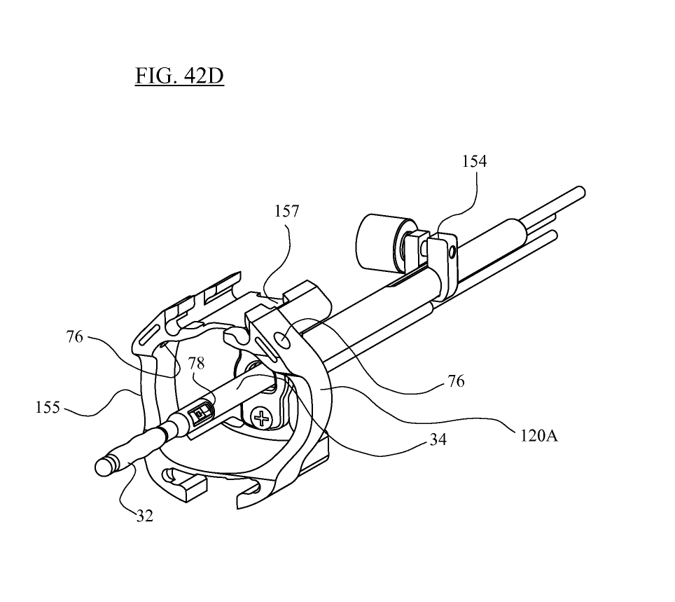

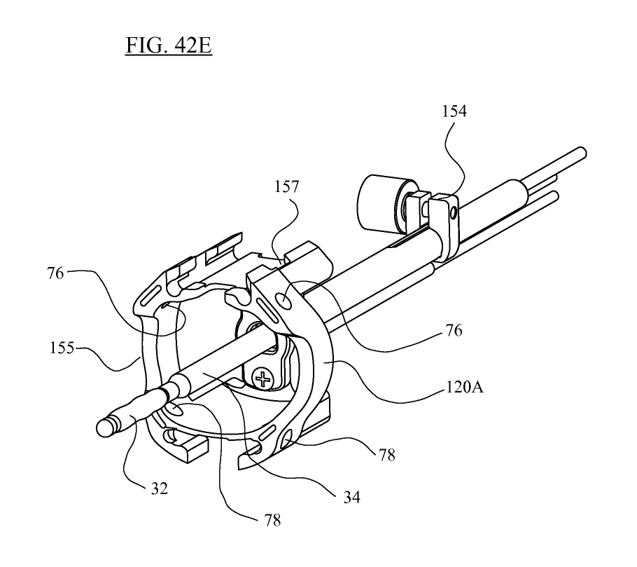

For some applications, at least a portion of oximeter 40 is disposed on fornix-engaging structure 24 (and/or a different fornix-engaging structure described herein, such as two-portion fornix-engaging structure 110, and/or three-portion fornix-engaging structure 120, both of which are described hereinbelow), e.g., as described hereinbelow with reference to FIGS. 42D-E.

For some applications, a physician modulates the pressure inside balloon 28 responsively to one or more of the parameters detected by the sensors. For example, the physician may modulate the pressure using a pump 50 (shown in FIG. 1) that is in fluid communication with balloon 28, in response to one or more of the parameters detected by the sensors (or a parameter derived therefrom) being displayed on an output unit. Alternatively, a control unit 52 (shown in FIG. 1) modulates the pressure of balloon 28 responsively to the detected parameters (for example, using an automatic pumping system), in order to achieve occlusion of the uterine artery. For some applications, the techniques described herein for monitoring and modulating the pressure of balloon 28 are used for monitoring and modulating the pressure of positioning-anchoring balloon 32. (It is noted that, although separate control units are shown in FIG. 1 for controlling the pressure in each of the balloons, for some applications a single control unit is used to monitor and/or modulate the pressure in two or more of the balloons.) For some applications, the techniques described herein for monitoring and modulating the pressure of balloon 28 are used for monitoring and modulating the pressure of balloons 55L and 55R (application not shown).

For some applications, a manual pump (e.g., a syringe, bellows, and/or a different type of pump) is used to pump each of left and right balloons 28L and 28R, and positioning-anchoring balloon 32 with a fluid (such as air, gas, or saline). An operator inflates the balloons manually, for example, in response to an output that is displayed on a monitor (not shown) of bed-side system 10. For some applications, each of left and right balloons 28L and 28R, and positioning-anchoring balloon 32 is in fluid communication with a single housing that provides separate access for the pump to each of left and right balloons 28L and 28R, and positioning-anchoring balloon 32. For some applications, the pump and the housing are configured for single use. Alternatively, the pump and housing are configured for multiple uses.

For some applications, control unit 52 determines that blood flow through the uterine artery has stopped by detecting that there is zero blood flow in the vicinity of a sensor. For some applications, the sensors described herein detect non-zero blood flow in the vicinity of the sensor, even when the portion of the uterine artery that is downstream of the occlusion is fully occluded. This is due to blood flow through other blood vessels in the vicinity, and/or due to blood motion in the portion of the artery that is upstream of the occlusion and that continues to empty and fill, even when the artery is occluded. For some applications, control unit 52 determines that blood flow through the uterine artery has stopped by detecting that a value that (a) is associated with the parameter detected by one of the sensors, and (b) is indicative of blood flow in the vicinity, has changed and then plateaued at a value indicating non-zero blood flow through at least one other of the blood vessels and/or due to blood flow through the portion of the uterine artery that is upstream of the occlusion.

For some applications, even when the value of the parameter plateaus, the value of the parameter still includes a cyclical time-varying component. As such, in the context of the present application, the meaning of the term “plateau” should be interpreted as including a value that may include a cyclical time-varying component, but that has changed and plateaued relative to an original value.

For some applications, the control unit determines that the uterine artery is occluded by determining that light detected by oximeter 40 indicates that a level of oxyhemoglobin in the vicinity of the subject's uterine artery has decreased and then plateaued at a non-zero value. This is indicative of the fact that blood-flow through the uterine artery has stopped, but there is non-zero blood flow through other blood vessels in the vicinity of the uterine artery and/or due to blood motion through the portion of the uterine artery that is upstream of the occlusion.

Alternatively, the control unit may determine that the uterine artery is occluded in response to a pulsating component of the balloon pressure that is detected by pressure sensor 48. Typically, when balloon 28 is inflated at site 30, or balloon 55 is inflated, initially a pulsating component of the pressure signal begins to be detected, and then the strength of the pulsating component increases, as the balloon makes contact with the uterine artery, or tissue that is adjacent to the uterine artery. Subsequently, the strength of the pulsating component decreases as the uterine artery becomes occluded. For some applications, when the uterine artery becomes fully occluded, pressure sensor 48 detects a non-zero pulsating component, due to blood flow through other blood vessels in the vicinity of the uterine artery and/or due to blood motion through the portion of the uterine artery that is upstream of the occlusion.

For some applications, balloon 28, balloon 32, and/or balloon 55 is made of a non-stretchable material, such as reinforced nylon, polyurethane, and/or a similar material, in order to facilitate accurate pressure measurements of the balloon by pressure sensor 48. During use, the non-stretchable balloon is configured not to reach its maximum volume, but rather to be volume restricted by the tissue in the vicinity of the balloon. Therefore, the pressure required to inflate the balloon is equal to the pressure in the volume-restricting surrounding tissue. Thus, the non-stretchable balloon facilitates monitoring the pressure of the tissue surrounding the balloon. For some applications, the occlusion of the uterine artery is monitored utilizing an electronic system similar to systems utilized in common blood pressure monitoring systems, in which a non-stretchable balloon is inflated while being volume restricted between the arm and an outer fabric sleeve. Typically, the non-stretchable balloon is inserted into the subject's body in a deflated state. Further typically, the non-stretchable balloon is folded during insertion of the balloon into the subject's body, in order to enable passage of the balloon through small incisions. It is noted that, typically, the deflated volume and longitudinal footprint of a non-stretchable balloon are larger than those of a stretchable balloon that has the same maximum volume. It is further noted that before a non-stretchable balloon is inflated, the tissue-contacting surface of the balloon is typically not smooth. Rather, the surface typically includes unopened folds of the material.

Alternatively, balloon 28, balloon 32, and/or balloon 55 is made of a stretchable material, such as latex, silicone, and/or similar materials. Typically, during the inflation of a stretchable balloon, a first portion of the inflation pressure is used to stretch the stretchable material. A second portion of the inflation pressure is associated with overcoming the pressure exerted on the balloon by the surrounding tissue that contacts the balloon. Thus, a stretchable balloon typically does not facilitate accurate blood pressure monitoring of the arteries that are in the vicinity of the balloon (although the measurements may in any case be sufficiently accurate for identifying occlusion). Using a stretchable balloon typically facilitates insertion of the balloon via a small incision, since the deflated volume and longitudinal footprint of the deflated stretchable balloon are small compared with those of a non-stretchable balloon. A stretchable balloon typically inflates in a generally smooth and uniform manner

For some applications, balloon 28, balloon 32, and/or balloon 55 is made from two materials. For example, a non-stretchable sheet may be welded to a stretchable sheet, so as to enable stretching of the balloon in a first direction (e.g., toward the uterine arteries), while inhibiting stretching of the balloon in a second direction (embodiment not shown). The scope of the present invention includes using for balloon 28, balloon 32, and/or balloon 55 any combination of stretchable, non-stretchable, stiff, and/or other types of materials, as would be apparent to one skilled in the art.

For some applications, one or more of balloons 28, 32, and 55 is made of a non-stretchable material, in order to facilitate accurate pressure measurements of the balloon by pressure sensor 48, and one or more of the other balloons of balloons 28, 32, and 55 is made of a stretchable material, such as latex.

For some applications, the control unit determines that the uterine artery is occluded, in response to a pulsating component of sound waves detected by microphone 46 decreasing in value and plateauing at a non-zero-value. For some applications, when the uterine artery becomes fully occluded, the microphone detects a non-zero pulsating component of the sound waves due to blood flow through other blood vessels in the vicinity of the uterine artery and/or due to blood motion through the portion of the uterine artery that is upstream of the occlusion.

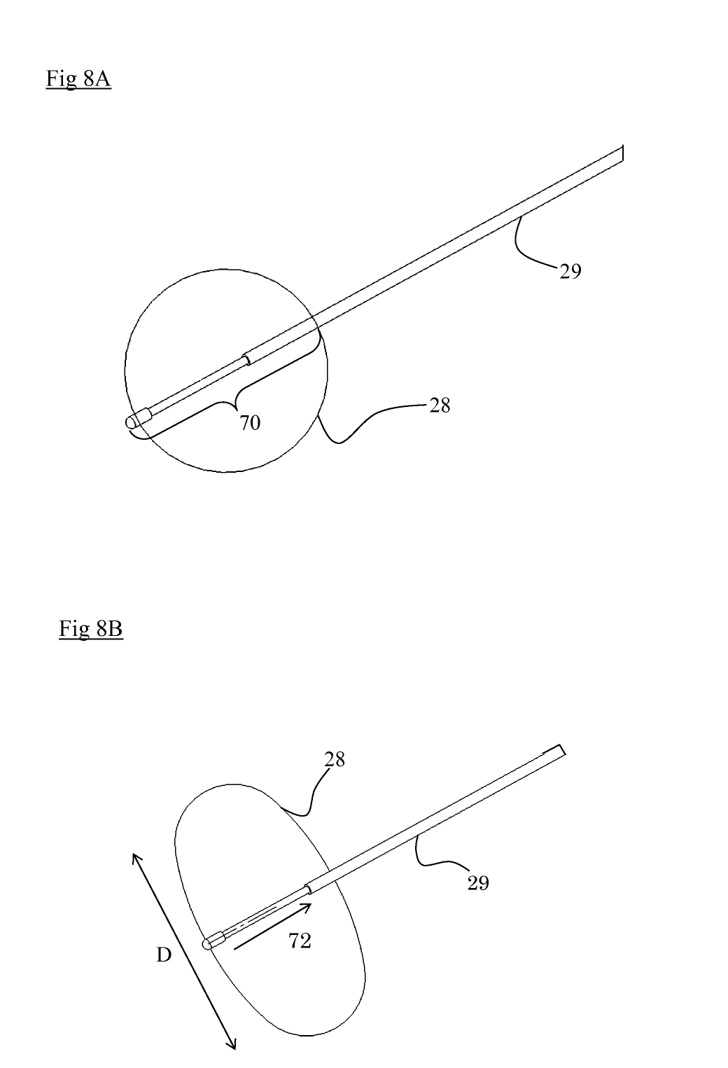

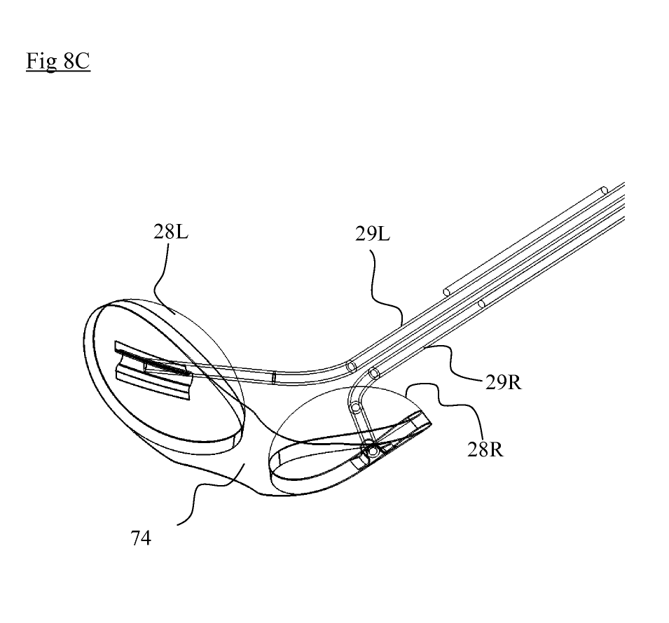

Reference is now made to FIGS. 8A-C, which are schematic illustrations of a balloon 28 for occluding the uterine artery, in accordance with an application of the present invention. For some applications, a balloon as is known in the prior art is used as uterine artery compression device 28 of device 20. In general, the scope of the present invention is not limited to using balloons as described in FIGS. 8A-C for the uterine artery compression device of device 20. It is further noted that the scope of the present invention includes using a balloon that is disposed in a non-coaxial position at the distal end of tube 29, to occlude that uterine artery.

For some applications, as shown in FIGS. 8A-B, tube 29 includes a telescoping portion 70 at a distal end thereof. As shown in FIG. 8A, balloon 28 is inflated. Then, as shown in FIG. 8B, the telescoping portion is shortened, for example, by the distal end of the tube being retracted proximally, in the direction of arrow 72. Typically, shortening the telescoping portion causes the balloon to become more elliptical, and for the diameter D of the balloon to increase in a direction that is perpendicular to the longitudinal axis of tube 29. For some applications, increasing the diameter of the balloon in this direction is performed in this manner, in order to further compress the uterine artery.

Reference is now made to FIG. 8C, which is a schematic illustration of left and right balloons 28, in accordance with some applications of the present invention. For some applications, the left and right balloons are inserted into the subject's body via a single vaginal incision, for example, at the 6 o'clock position, as described with reference to FIG. 3A-E. For some applications, the balloons are coupled to a flexible connecting member 74 that is non-stretchable, and that couples the two balloons to each other, as shown in FIG. 8C. For some applications, connecting member 74 defines a maximum distance between respective centers of the left and right uterine balloons of, for example 5-25 cm, e.g., 12 cm. Alternatively, connecting member 74 is stretchable, or comprises a combination of stretchable and non-stretchable materials. For some applications, connecting member 74 is made of reinforced nylon, polyurethane, and/or a similar material. The connecting member typically ensures that the inflation of the balloons is substantially on the other side of each of the balloons to the side that is coupled to the connecting member. Furthermore, the connecting member typically correctly positions each of the balloons with respect to the other balloon, as described in further detail hereinbelow. For some applications, a plurality of pieces of connecting member 74 (e.g. 2-10 strips of connecting member 74) connect the left and right balloons. Alternatively, a single connecting member connects the left and right balloons.

Although applications are described herein relating to using a balloon on a telescoping pole for uterine artery compression, the scope of the present invention includes using a balloon on a telescoping pole, as described herein, for a different purpose, e.g., to compress a different artery, or organ.

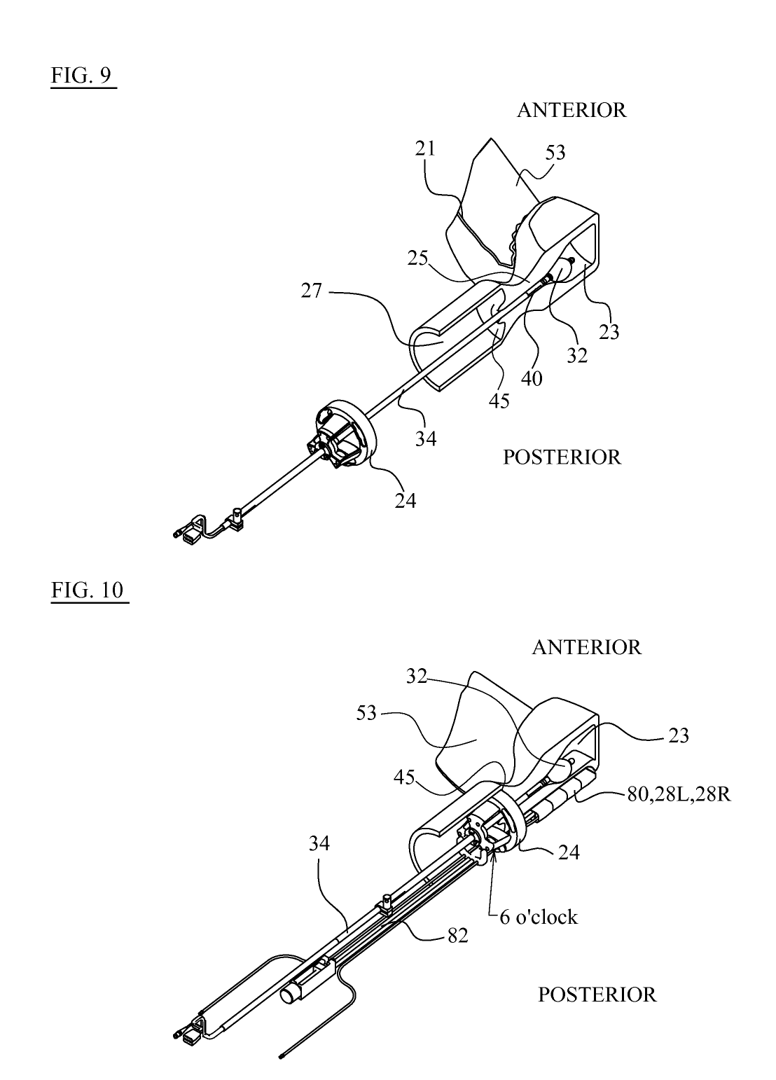

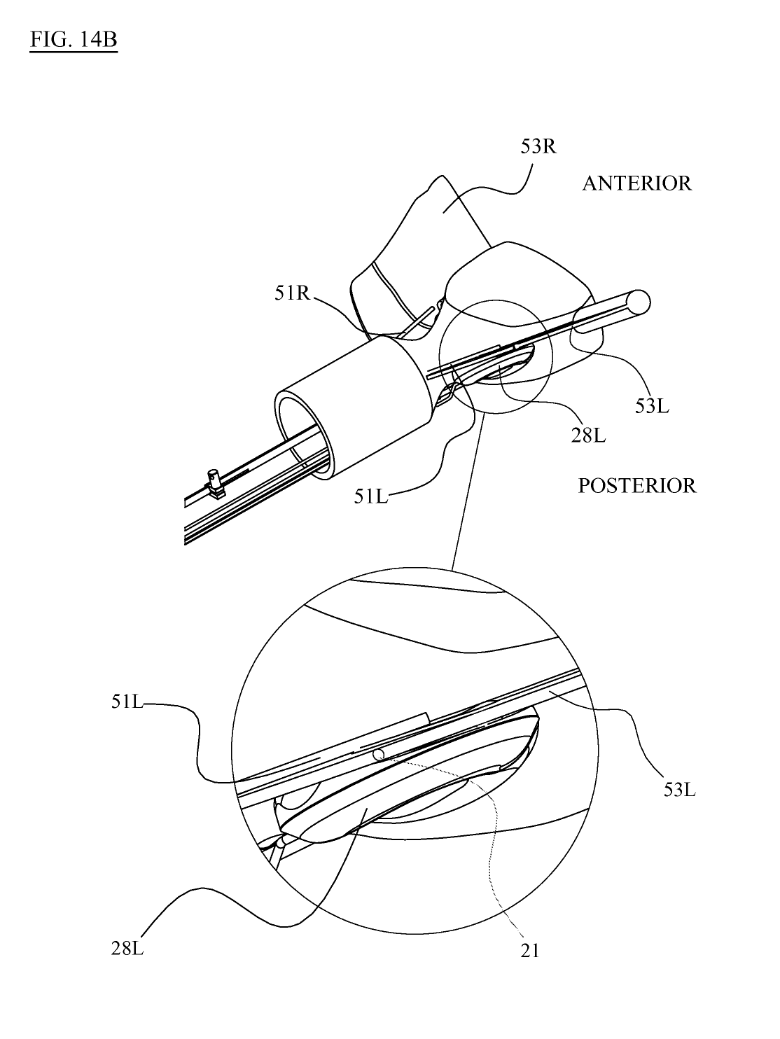

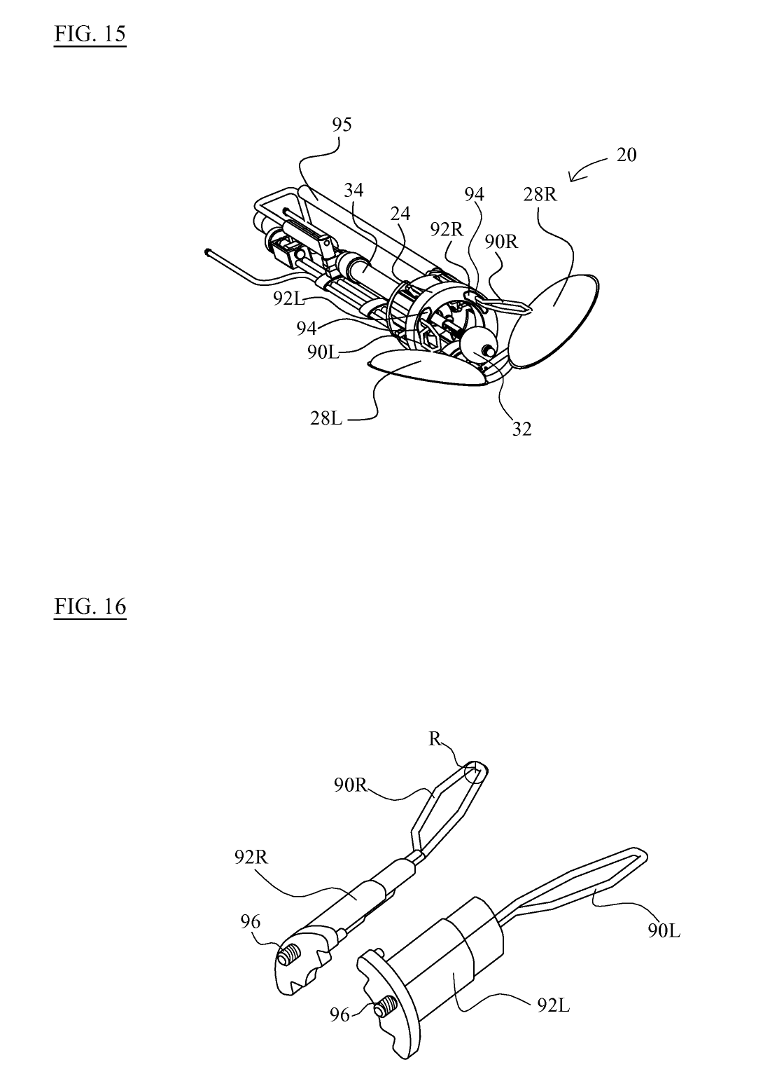

Reference is now made to FIGS. 9-14, which are schematic illustrations of respective steps of a procedure for occluding a subject's uterine arteries 21 using device 20 (shown in an assembled configuration thereof in FIG. 15), in accordance with some applications of the present invention. Fornix-engaging structure 24 of device 20, as shown in FIGS. 9-14, is generally similar to fornix-engaging structure 24 described hereinabove. However, fornix-engaging structure 24, as shown in FIGS. 9-14, comprises a guiding structure, for example, for guiding an insertion structure 82 of the device.

In a first step, shown in FIG. 9, a positioning-anchoring balloon 32, which is disposed at the distal end of positioning-anchoring tube 34, is inserted into the subject's uterus 23, via the subject's cervix 25 and vagina 27. The positioning-anchoring balloon and tube are used to stabilize the fornix-engaging structure, in accordance with the techniques described herein and in US Patent Application 2011/0022073 to Gross, which is incorporated herein by reference. Typically, positioning-anchoring tube 34 includes at least one oximeter 40 (described with reference to FIG. 7, for example) for monitoring the level of oxyhemoglobin in the vicinity of the subject's uterine arteries, uterus, and/or cervix, in accordance with the techniques described herein and in US Patent Application 2011/0022073 to Gross, which is incorporated herein by reference. Positioning-anchoring balloon 32 is inflated inside the subject's uterus, as shown.

In a second step, shown in FIG. 10, and subsequent to the inflation of positioning-anchoring balloon 32, fornix-engaging structure 24 is advanced over positioning-anchoring tube 34 into the subject's fornix 45. When the fornix-engaging structure is positioned in a desired intra-procedural position (i.e., inside and abutting the subject's fornix), the position of the fornix cap is typically locked (longitudinally and angularly), with respect to positioning-anchoring tube 34. An incision is made in the subject's fornix, at approximately a 6 o'clock position, i.e., on the posterior side of fornix 45. For some applications, incisions are additionally made at approximately 2 o'clock and 10 o'clock positions in the subject's fornix, for reasons described hereinbelow.

Left and right balloons 28L and 28R are inserted through tissue into a position posterior to cervix 25, via the 6 o'clock incision in the subject's fornix. Typically, the balloons are inserted while the balloons are (a) in deflated states, and (b) both inside of a cover 80. Further typically, the balloons are disposed adjacent to one another, inside the cover, or at another suitable relative disposition with respect to one another, such that the balloons can be inserted simultaneously through a single 6 o'clock incision in the fornix. An insertion assembly 82 is used to insert the balloons, the insertion assembly being guided by a guide. In accordance with respective applications, the guide for guiding insertion assembly 82 is defined by the fornix-engaging structure (as shown), or is coupled to the fornix-engaging structure. When balloons 28L and 28R are positioned at a desired position, posterior to the subject's cervix, insertion assembly 82 is locked with respect to positioning-anchoring tube 34. For some applications, insertion assembly 82 is locked with respect to fornix-engaging structure 24.

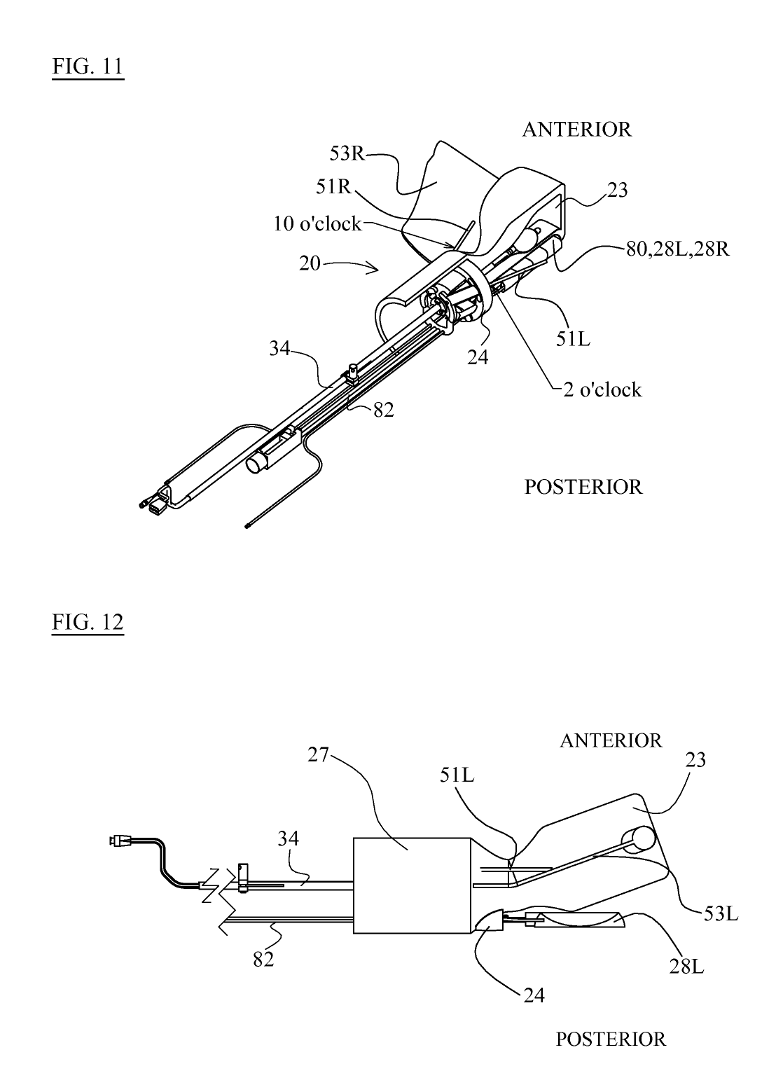

In a third step, shown in FIG. 11, left and right rigid structures 51L and 51R are inserted through, respectively, the 2 o'clock incision and the 10 o'clock incision in the subject's fornix. As an alternative to incising the fornix at approximately 2 o'clock and 10 o'clock positions in a separate step, for some applications, the distal ends of left and right rigid structures 51L and 51R are sharp, and are used to penetrate the vaginal tissue by forming incisions in the vaginal tissue. The left and right rigid structures are inserted through the subject's vaginal tissue to positions, respectively, anterior to left broad ligament 53L (shown in FIG. 12) and right broad ligament 53R.

In a fourth step, shown in FIG. 12, cover 80 (shown covering the balloons in FIG. 11) is withdrawn from balloons 28L and 28R. For example, the cover may be withdrawn by pulling a string that is coupled to the cover, as described hereinbelow. For some applications, instead of cover 80 being withdrawn from balloons 28L and 28R, the cover is torn along its length, for example, along a pre-perforated line. Alternatively, the cover is removed from the balloons by another mechanism. Typically, the cover is flexible, and may be, for example, a sheath.

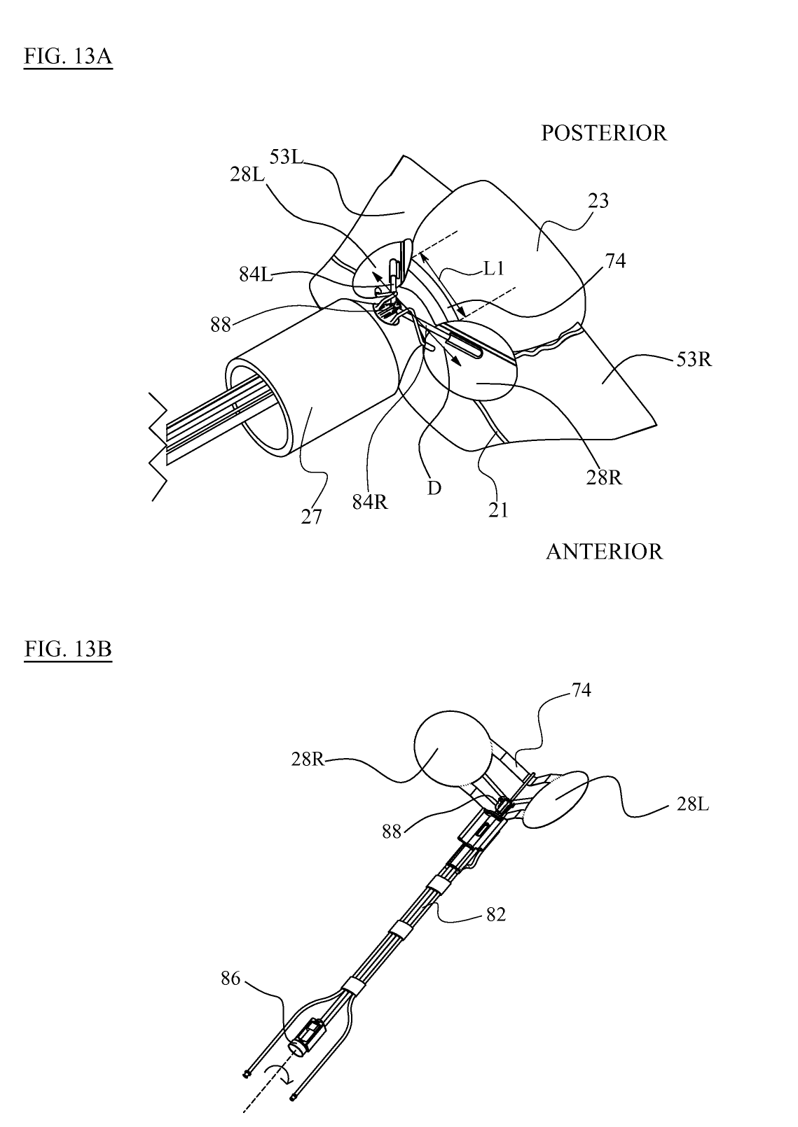

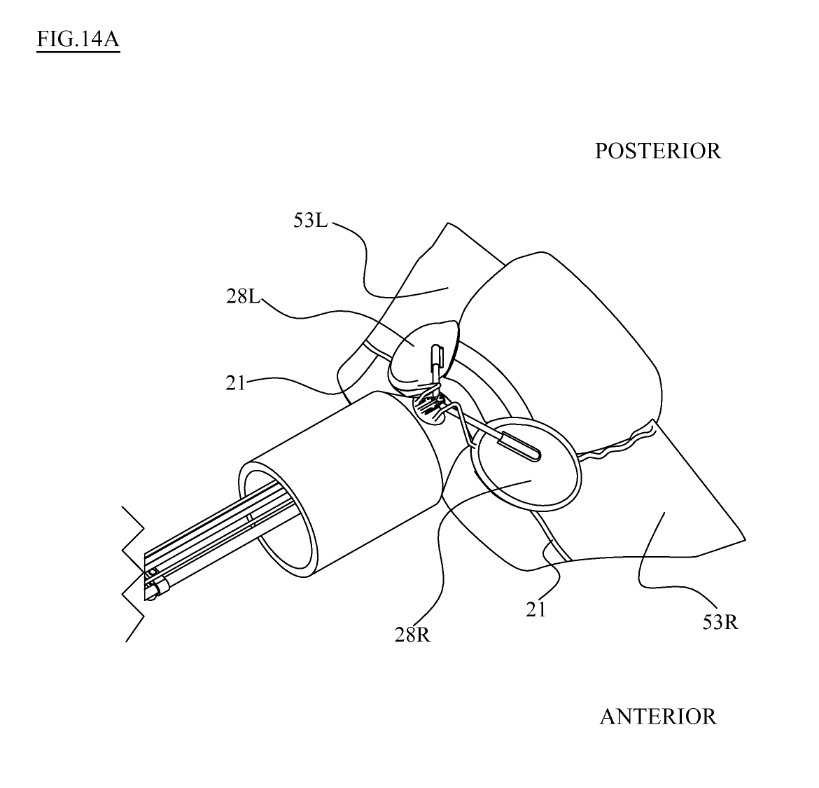

In a fifth step shown in FIGS. 13A-B, left and right rods 84L and 84R, to which balloons 28L and 28R are connected, are lifted anteriorly and are separated from each other by being moved respectively to left and right sides of the cervix. For some applications, in order to separate the rods, a knob 86 (shown in FIG. 13B) is turned, the knob being disposed outside the subject's body. The turning of the knob actuates a mechanism 88 to separate the rods 84L and 84R from one another by moving the rods sideways, and to lift the rods anteriorly. The lifting and separation of rods 84L and 84R is such that, subsequent to the lifting and separation, balloons 28L and 28R are positioned posterior to, respectively, left and right broad ligaments 53L and 53R.

As can be observed in FIG. 13A, left and right balloons 28L and 28R are typically coupled to each other by a flexible connecting member 74, for example, as described hereinabove. Typically, the flexible material defines the maximum distance D between the centers of the left and right balloons. For some applications, the flexible material is sized such that the maximum distance between the centers is 25-200 mm, e.g., 40-80 mm. For example, the length L1 of the flexible material may be 5-60 mm. As described hereinabove, for some applications, one or a plurality of pieces of connecting member 74 connect the left and right balloons. The connecting member typically stabilizes the positions of the balloons with respect to one another.

For some applications, connecting member 74 is stretchable, or comprises a combination of stretchable and non-stretchable materials. Typically, connecting member 74 is made of polyurethane, reinforced nylon, and/or a similar material. Typically, the connecting member facilitates correct positioning of each of the balloons with respect to the other balloon and restricts the balloons from rotating around rods 84 during inflation of the balloons, as described in further detail hereinbelow.

In a sixth step, respective views of which are shown in FIGS. 14A and 14B, balloons 28L and 28R are inflated. The inflated balloons press the subject's broad ligaments 53L, 53R against rigid structures 51L, 51R (shown in FIG. 14B), thereby compressing and occluding the subject's uterine arteries.

Reference is now made to FIGS. 15-16, which are schematic illustrations of device 20 including non-penetrating rigid structures 90L and 90R for facilitating the occlusion of a subject's uterine arteries, in accordance with some applications of the present application. Typically, the non-penetrating rigid structures perform a generally similar function to rigid structures 51L and 51R, described hereinabove. That is to say that the non-penetrating rigid structures are positioned anterior to the subject's broad ligaments and provide resistance and/or clamping support against which a uterine artery compression device (e.g., balloons 28L and 28R described hereinabove) presses the subject's broad ligaments. However, the non-penetrating rigid structures differ from rigid structures 51L and 51R in that the non-penetrating rigid structures are positioned anterior to the subject's broad ligaments, without penetrating (or otherwise passing through) vaginal tissue. Rather the non-penetrating rigid structures stretch the fornix into positions anterior to the subject's broad ligaments.

For some applications, non-penetrating rigid structures 90L and 90R have distal portions that are curved. For example, the radius of curvature R of the distal portions may be 1-20 mm, e.g., 2-10 mm.

Typically, in procedures in which non-penetrating rigid structures 90L and 90R are used, the incisions in the vaginal fornix at the 2 o'clock and 10 o'clock positions, described with reference to FIG. 10, are not made. Rather, the non-penetrating rigid structures are pushed distally into the vaginal fornix at the approximately 2 o'clock and 10 o'clock positions, such that they are positioned anterior to the left and right broad ligaments, while still within the vaginal fornix, by stretching the vaginal fornix.

Typically, a guide is used to guide non-penetrating rigid structures 90L and 90R to the 2 o'clock and 10 o'clock positions. For some applications, the non-penetrating rigid structures are supported by support structures 92. The support structures are shaped to match portions of fornix-engaging structure 24. For example, fornix-engaging structure 24 may define guides 94 that are shaped to guide the support structures, and thereby guide the non-penetrating rigid structures into the 2 o'clock and 10 o'clock positions. Alternatively, a guiding structure may be couplable to the fornix-engaging structure for guiding the non-penetrating rigid structures into position, as described hereinabove.

For some applications, a rigid-structure-insertion-rod 95 is used to insert non-penetrating rigid structures 90L and 90R into the vaginal fornix. For example, as shown in FIG. 16, a threaded portion 96 of non-penetrating rigid structures 90L and 90R may protrude from support structures 92. During insertion of each of the non-penetrating rigid structures into the vaginal fornix, rigid-structure-insertion-rod 95 is screwed onto threaded portion 96. The rigid-structure-insertion-rod is used to push support structure 92 through guide 94, thereby placing the non-penetrating rigid structure in its intra-procedural position. Subsequent to the placement of the non-penetrating rigid structure, the rigid-structure-insertion-rod is unscrewed from threaded portion 96 of the non-penetrating rigid structure, and removed from the subject's body.

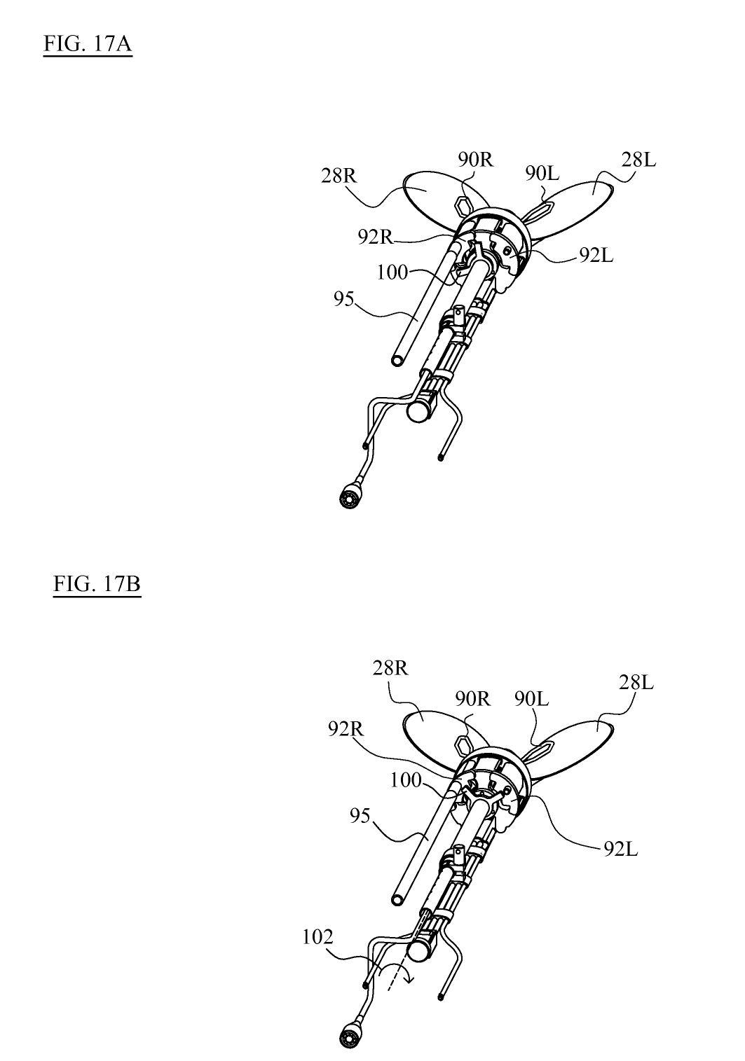

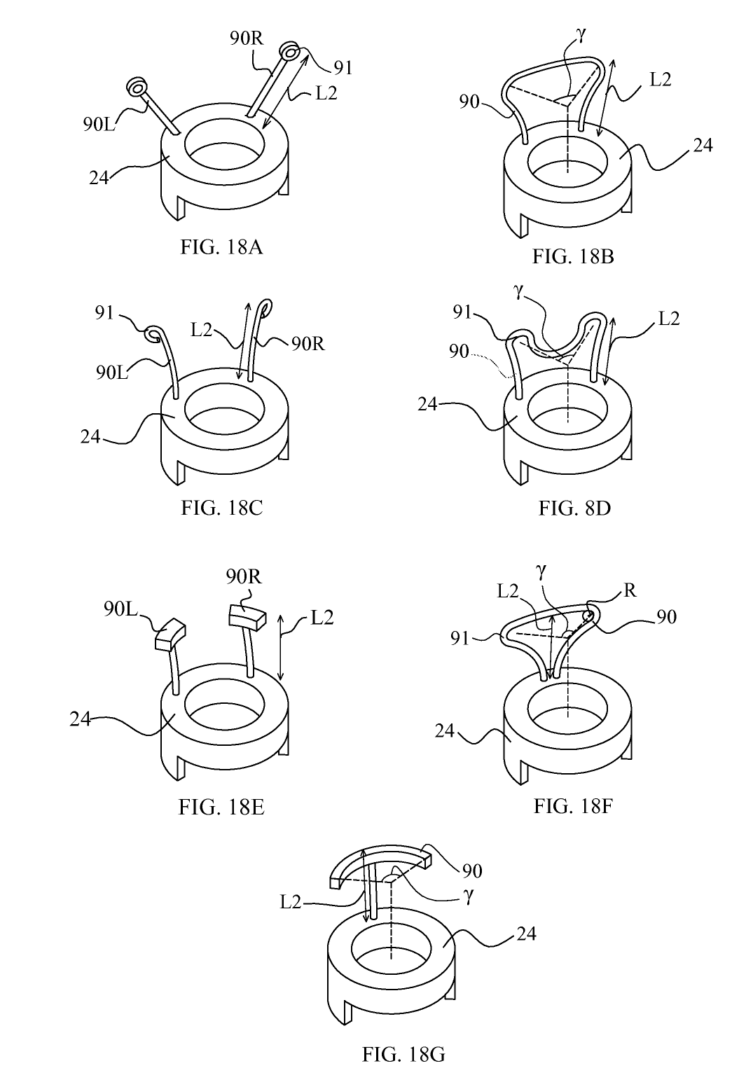

Reference is now made to FIGS. 17A-B, which are schematic illustrations of a locking mechanism 100, respectively, in unlocked and locked states, in accordance with some applications of the present invention. Typically, during insertion of non-penetrating rigid structures 90L and 90R, via fornix-engaging structure 24, locking mechanism 100 is in an unlocked state thereof, as shown in FIG. 17A. When both of the non-penetrating rigid structures 90L and 90R are positioned within the vaginal fornix (e.g., at the 2 o'clock and 10 o'clock positions), locking mechanism 100 is rotated in the direction of arrow 102, such that the locking mechanism locks support structures 92 into place, thereby locking non-penetrating rigid structures 90L and 90R into place.

For some applications, the locking procedure described above (or another procedure) is performed in order to lock non-penetrating rigid structures 90L and 90R. This is because non-penetrating rigid structures 90L and 90R are pushed distally into the vaginal fornix such that structures 90L and 90R are positioned anterior to the subject's broad ligaments, as described hereinabove. Due to the elasticity of the vaginal fornix, the fornix has a tendency to push non-penetrating rigid structures 90L and 90R proximally. Therefore, for some applications, locking mechanism 100 is used to maintain the position of non-penetrating rigid structures 90L and 90R inside the vaginal fornix, as described hereinabove.

For some applications, locking mechanism 100, or a similar mechanism, is used to lock penetrating rigid structures 51L and 51R, described hereinabove, into position.

For some applications, penetrating rigid structures 51L and 51R, and/or non-penetrating rigid structures 90L and 90R are locked into position with respect to fornix-engaging structure 24 (e.g., using locking mechanism 100), before being inserted into the subject's vagina. (Optionally, the rigid structures are originally assembled in one integral piece with the fornix-engaging structure.) In such applications, the fornix-engaging structure is inserted into the fornix with the rigid structures coupled thereto. Subsequent to being inserted into the fornix, the fornix-engaging structure is locked with respect to positioning-anchoring tube 34, as described hereinabove.

Reference is now made to FIGS. 18A-G, which are schematic illustrations of non-penetrating rigid structures 90, in accordance with respective applications of the present invention. For some applications, instead of using non-penetrating rigid structures that are shaped as shown in FIGS. 15-16, one or more of the non-penetrating rigid structures shown in FIGS. 18A-G are used, mutatis mutandis.

Although non-penetrating rigid structures are shown having given shapes in FIGS. 15-16 and FIGS. 18A-G, the scope of the present invention includes non-penetrating rigid structures having different shapes that would perform a similar function to the non-penetrating rigid structures that are shown in the aforementioned figures.

For some applications the distal portions of the non-penetrating rigid structures shown in FIGS. 15-16 and/or FIGS. 18A-G are coated with a soft coating, in order to prevent the distal portions from penetrating tissue of the subject.

The dimensions of the non-penetrating rigid structures that are shown in FIGS. 18A-G are typically as follows:

Length L2 of non-penetrating rigid structures 90L and 90R, shown in FIG. 18A, is typically up to 80 mm. The radius of curvature of curved distal portions 91 of each of the structures is typically 5 mm to 15 mm.

Length L2 of non-penetrating rigid structure 90, shown in FIG. 18B, is typically up to 80 mm It is noted that for some applications, as shown in FIG. 18B, a single rigid structure is used as both the left and right rigid structures. That is to say that a single rigid structure is placed anterior to both the subject's left and right broad ligaments. The rigid structure typically defines an angle gamma, with respect to the longitudinal axis of fornix-engaging structure 24, of 70 degrees to 160 degrees. The angle gamma is the angle about the longitudinal axis that is encompassed by the angular extremities of rigid structure 90.

Length L2 of non-penetrating rigid structures 90L and 90R, shown in FIG. 18C, is typically up to 80 mm. The radius of curvature of curved distal portions 91 of each of the structures is typically 5 mm to 15 mm.

Length L2 of non-penetrating rigid structure 90, shown in FIG. 18D, is typically up to 80 mm. The radius of curvature of curved distal portions 91 of the structure is typically 5 mm to 25 mm. The rigid structure typically defines an angle gamma, with respect to the longitudinal axis of fornix-engaging structure 24, of 70 degrees to 160 degrees. The angle gamma is the angle about the longitudinal axis that is encompassed by the angular extremities of rigid structure 90.

Length L2 of non-penetrating rigid structures 90L and 90R, shown in FIG. 18E, is typically up to 80 mm

Length L2 of non-penetrating rigid structure 90, shown in FIG. 18F, is typically up to 80 mm. The radius of curvature R of curved distal portions 91 of the structure is typically 5 mm to 25 mm. The rigid structure typically defines an angle gamma, with respect to the longitudinal axis of fornix-engaging structure 24, of 70 degrees to 160 degrees. The angle gamma is the angle about the longitudinal axis that is encompassed by the angular extremities of rigid structure 90.

Length L2 of non-penetrating rigid structure 90, shown in FIG. 18G, is typically up to 80 mm. The rigid structure typically defines an angle gamma, with respect to the longitudinal axis of fornix-engaging structure 24, of 70 degrees to 160 degrees. The angle gamma is the angle about the longitudinal axis that is encompassed by the angular extremities of rigid structure 90.

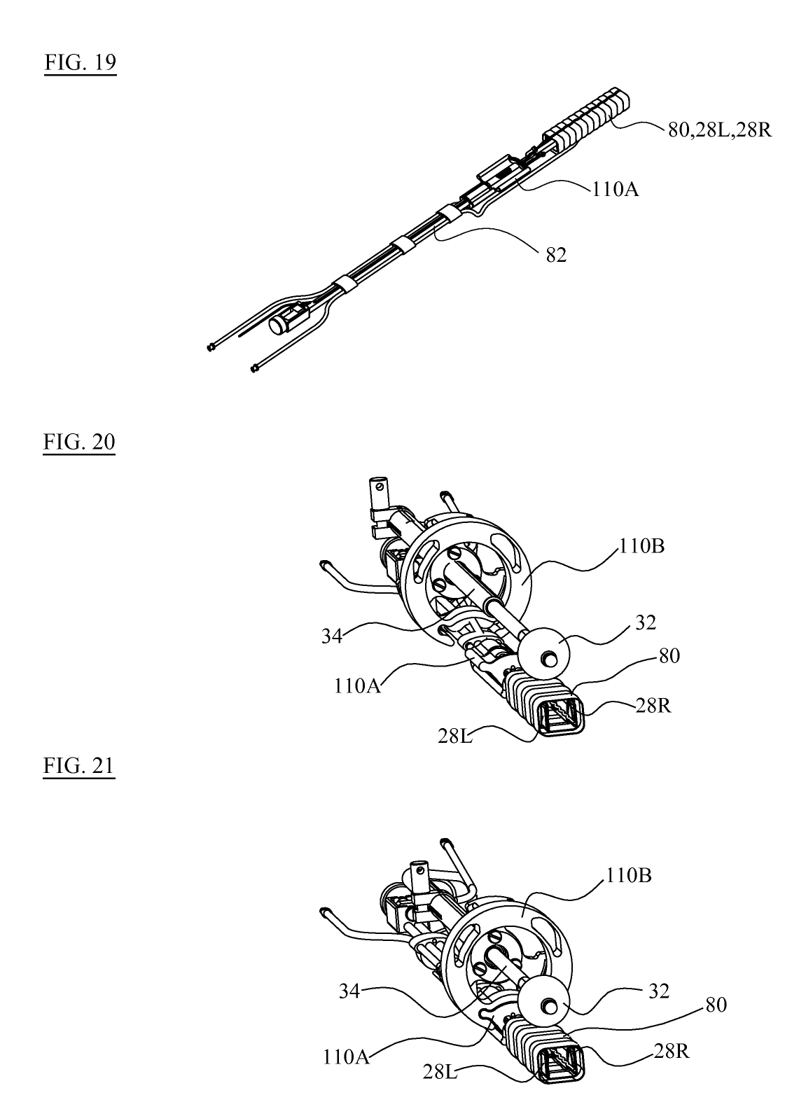

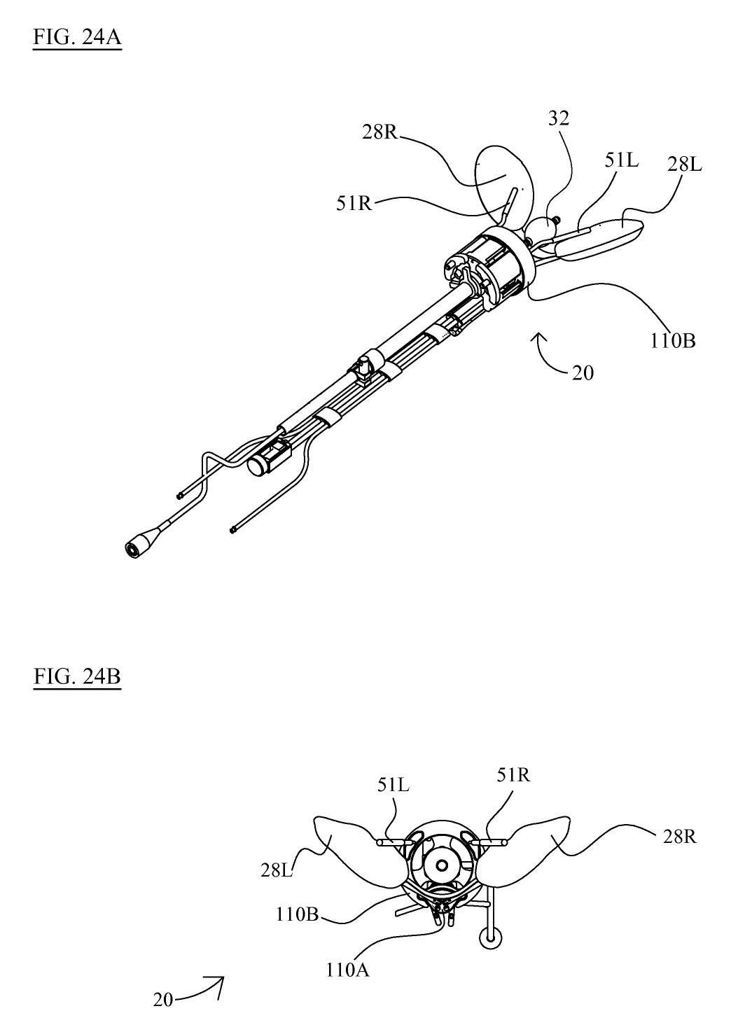

Reference is now made to FIGS. 19-24, which are schematic illustrations of respective steps of a procedure for occluding a subject's uterine arteries using device 20 (shown in an assembled configuration thereof in FIG. 24A, for example) that includes a two-portion fornix-engaging structure 110, in accordance with some applications of the present invention. It is noted that, for clarity, FIGS. 19-24 show the steps of the procedure without showing the portions of the subject's anatomy with respect to which the procedure takes place. Fornix-engaging structure 110 includes a first portion 110A and a second portion 110B. First and second portions 110A and 110B are inserted into the subject's fornix, in accordance with the techniques described hereinbelow, such that, in combination with one another, the first and second portions engage the vaginal fornix. The two-portion fornix-engaging structure and techniques for use therewith are generally similar to fornix-engaging structure 24 and the techniques for use therewith described hereinabove, mutatis mutandis. However, fornix-engaging structure 24 typically includes a single portion that engages the vaginal fornix.

In a first step, shown in FIG. 19, first portion 110A of the fornix-engaging structure is inserted into the subject's fornix (fornix not shown), through an incision, as described hereinbelow. First portion 110A of the fornix-engaging structure is typically coupled to insertion assembly 82, which is generally similar to insertion assembly 82, described with reference to FIG. 10. Further typically, left and right balloons 28L and 28R and cover 80, described hereinabove with reference to FIG. 10, are disposed on the distal end of insertion assembly 82. Alternatively or additionally, a different uterine artery compression device is disposed on the distal end of insertion assembly 82.

In a typical application, prior to first portion 110A of the fornix-engaging structure being inserted, an incision is made at approximately the 6 o'clock position in the vaginal fornix. Subsequently, insertion assembly 82 is inserted into the vaginal fornix, such that (a) balloons 28L and 28R pass through the incision into a position posterior to the subject's cervix, and (b) first portion 110A of the fornix-engaging structure engages the posterior side of the vaginal fornix.

Subsequently, positioning-anchoring balloon 32 is inserted into the subject's uterus and inflated. Positioning-anchoring balloon 32 is disposed at the distal end of positioning-anchoring tube 34. Positioning-anchoring balloon 32 and positioning-anchoring tube 34 are generally as described hereinabove. The positioning-anchoring balloon is typically inserted into the subject's uterus via second portion 110B of the fornix-engaging structure, prior to second portion 110B engaging the fornix. Further subsequently, portion 110B of the fornix-engaging structure is advanced over positioning-anchoring tube 34, into contact with the vaginal fornix (shown in FIGS. 20 and 21, without the fornix). Portions 110A and 110B become coupled to each other, as shown in FIG. 21, inside the vaginal fornix (fornix not shown). When the two-portion fornix-engaging structure is positioned in a desired intra-procedural position, i.e., inside the subject's fornix, the position of the fornix cap is typically locked with respect to positioning-anchoring tube 34, e.g., in accordance with the techniques described herein.

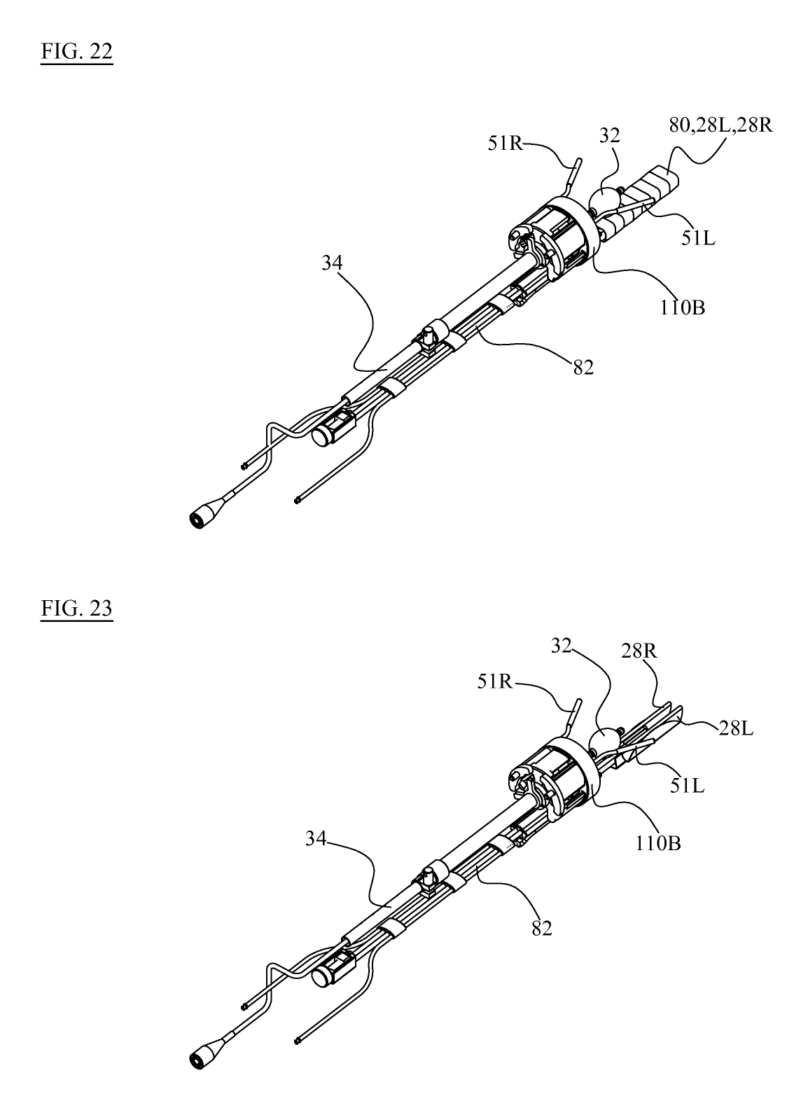

Subsequently, incisions are made at approximately 2 o'clock and 10 o'clock positions in the vaginal fornix. Rigid structures 51L and 51R are inserted through the 2 o'clock and 10 o'clock incisions to positions anterior to, respectively, the subject's left and right broad ligaments. For some applications the rigid structures are guided into the aforementioned positioned by portion 110B of the fornix-engaging structure, using generally similar techniques to those described hereinabove. FIG. 22 shows device 20 at this stage.

It is noted that although FIG. 22 shows rigid structures 51L and 51R, which pass through incisions in the subject's vaginal tissue, for some applications, non-penetrating rigid structures are inserted into the approximately 2 o'clock and 10 o'clock positions in the vaginal fornix. For example, non-penetrating rigid structures 90L and 90R, described hereinabove, may be used, using generally similar techniques to those described hereinabove.

As shown in FIG. 23, subsequent to rods 51L and 51R having been inserted into their positions anterior to the subject's broad ligaments, cover 80 is withdrawn from balloons 28L and 28R. This step is generally similar to the cover-withdrawal step described with reference to FIG. 12.

In a subsequent step, shown in FIGS. 24A-B, balloons 28L and 28R are (a) lifted anteriorly and separated sideways from one another, using a technique that is generally similar to that described with reference to FIGS. 13A-B, and (b) inflated such that the balloons press the left and right broad ligaments against rigid structures 51L and 51R (or non-penetrating rigid structures 90L and 90R).

FIG. 24B shows a posterior-cephalic view of the fornix-engaging structure, in which the coupling of first and second portions 110A and 110B of the fornix-engaging structure may be observed. As noted hereinabove, the first and second portions are typically coupled to each other inside the vaginal fornix.

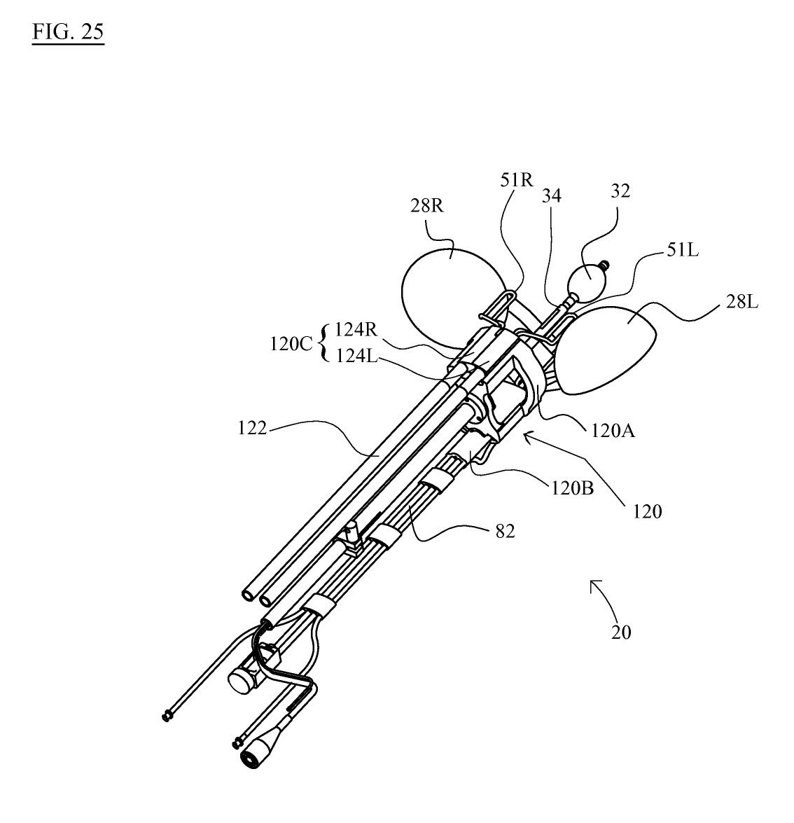

Reference is now made to FIG. 25, which is a schematic illustration of device 20 including a three-portion fornix-engaging structure 120, in accordance with some applications of the present invention. First, second, and third portions 120A, 120B, and 120C are inserted toward the subject's fornix, in accordance with the techniques described hereinbelow, such that, in combination with one another, the first, second, and third portions engage the vaginal fornix. The three-portion fornix-engaging structure and techniques for use therewith are generally similar to fornix-engaging structure 24 and the techniques for use therewith described hereinabove, mutatis mutandis. However, fornix-engaging structure 24 typically includes a single portion that engages the vaginal fornix. The three-portion fornix-engaging structure and techniques for use therewith are generally similar to fornix-engaging structure 110 and the techniques for use therewith described hereinabove, mutatis mutandis. However, fornix-engaging structure 110 typically includes two portions that engage the vaginal fornix.

As shown in FIG. 25, positioning-anchoring tube 34 and positioning-anchoring balloon 32 are inserted via first portion 120A of fornix-engaging structure 120. Left and right balloons 28L and 28R are coupled to second portion 120B of fornix-engaging structure 120. Second portion 120B is inserted into the fornix using insertion assembly 82, as described hereinbelow. Rigid structures (e.g., penetrating rigid-structures 51L and 51R) are coupled to third portion 120C of fornix-engaging structure 120. As described hereinbelow, the third portion of the fornix-engaging structure is typically inserted into the fornix using decouplable insertion rods 122. The third portion of the fornix-engaging structure typically comprises left and right support elements 124L and 124R that support rigid structures 51L and 51R.

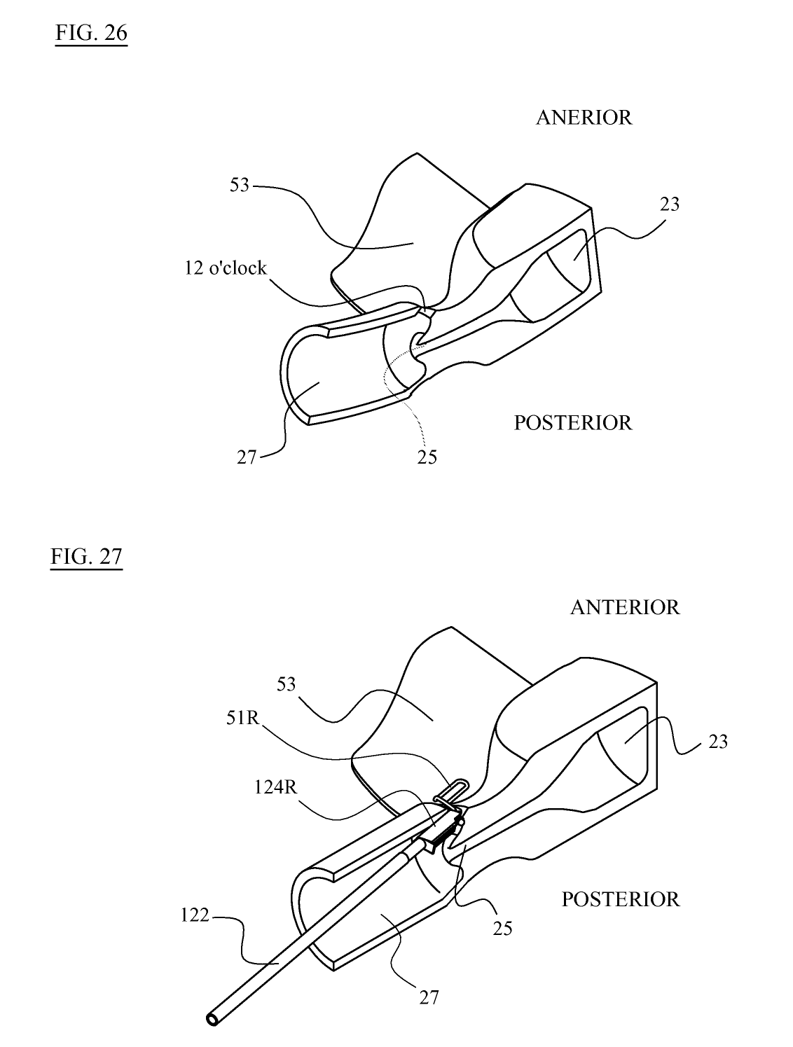

It is noted that although FIG. 25 shows rigid structures 51L and 51R that are shaped in shapes similar to non-penetrating rigid structures 90L and 90R (described hereinabove), in the technique described hereinbelow, rigid structures 51L and 51R do pass through an incision made in vaginal tissue into the paracervical space. Nevertheless, the scope of the present invention includes using non-penetrating rigid structures 90 for the technique described with reference to FIGS. 26-39, and/or using penetrating rigid structures 51 that have shapes that are generally similar to those shown in FIGS. 3A-F, mutatis mutandis.

Reference is now made to FIGS. 26-39, which are schematic illustrations of respective steps of a procedure that is used with three-portion fornix-engaging structure 120, in accordance with some applications of the present invention. The procedure typically includes one or more of the following steps:

(1) Bed-side system 10 (shown in FIG. 1) is prepared for the procedure. The bed-side system typically includes control unit 52, which typically controls inflation of balloons of device 20. Further typically, the bed-side system includes a monitor (not shown) that displays the readings of sensors of the system (e.g., pressure sensors 48, microphones 46, and/or oximeters 40, described hereinabove). For example, the preparation of the bed-side system may include the step of connecting sensors of the system (e.g., pressure sensors 48, microphones 46, and/or oximeters 40, as described hereinabove) to control unit 52 of the bed-side system, typically via cables. In addition, the preparation of the bed-side system may include the step of connecting inflation tubes of balloons of the system to a pressure-generating system, the pressure-generating system typically being under the control of control unit 52, and/or a healthcare provider.

(2) Measurements of the subject's anatomy are taken. For example, the subject's cervical width may be determined using either abdominal or transvaginal ultrasound. For some applications, dimensions of the rigid structures (e.g., penetrating rigid structures 51, and/or non-penetrating rigid structures 90) to be used during the procedure are selected based upon the measured cervical width. Alternatively or additionally, dimensions of the subject's fornix are measured, e.g., using a transvaginal sonogram. For some applications, dimensions of the fornix-engaging structure that is used during the procedure are selected based upon the measured dimensions of the subject's fornix.

(3) An anterior colpotomy (i.e., an incision at approximately the 12 o'clock position) is performed. FIG. 26 is a schematic illustration of a portion of the subject's fornix 45, an anterior colpotomy having been performed on the subject's fornix.

(4) Rigid structures are inserted into the paracervical space, via the anterior colpotomy. Typically, the rigid structures are temporarily inserted into the paracervical space at this stage (a) in order to develop and create paracervical space, and/or (b) in order to determine suitable dimensions of the rigid structures.

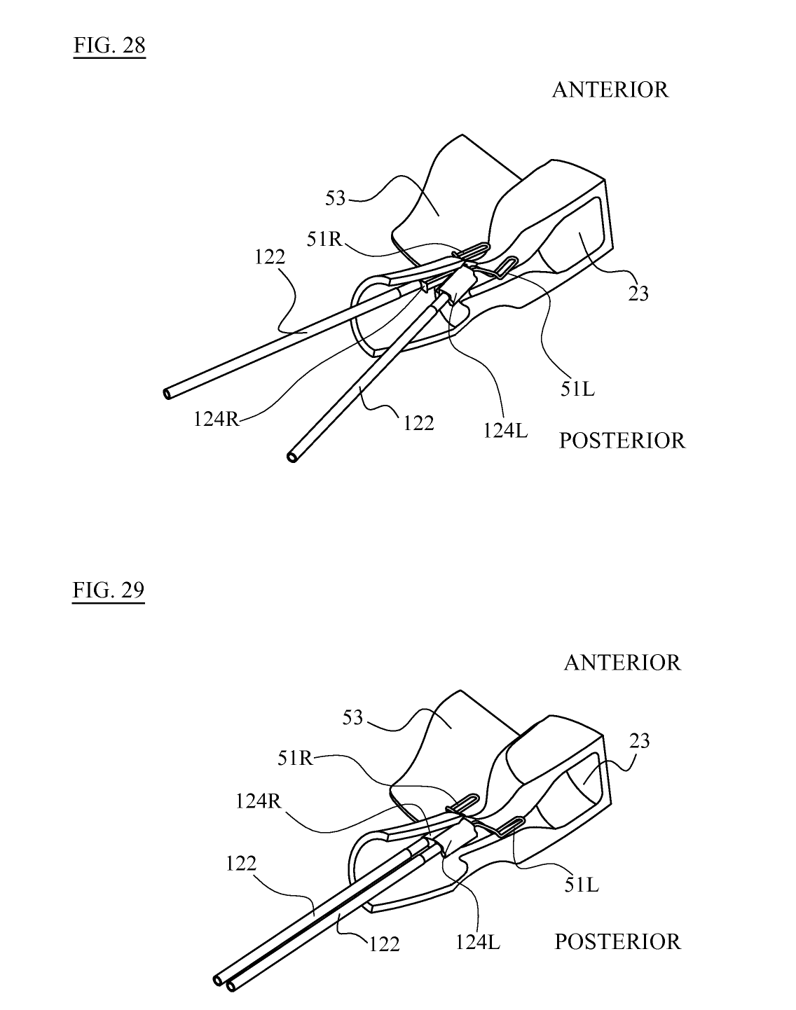

As shown in FIG. 27, a first one of the rigid structures (e.g. right rigid structure 51R, as shown) is inserted into the paracervical space via the anterior colpotomy, using insertion rod 122. Subsequently, a second one of the rigid structures (e.g. left rigid structure 51L) is inserted into the paracervical space, as shown in FIG. 28. Left and right support elements 124L and 124R are then coupled to each other, as shown in FIG. 29. Having developed the paracervical space, and/or having determined suitable dimensions of the rigid structure, the rigid structures are removed from the paracervical space using insertion rods 122.

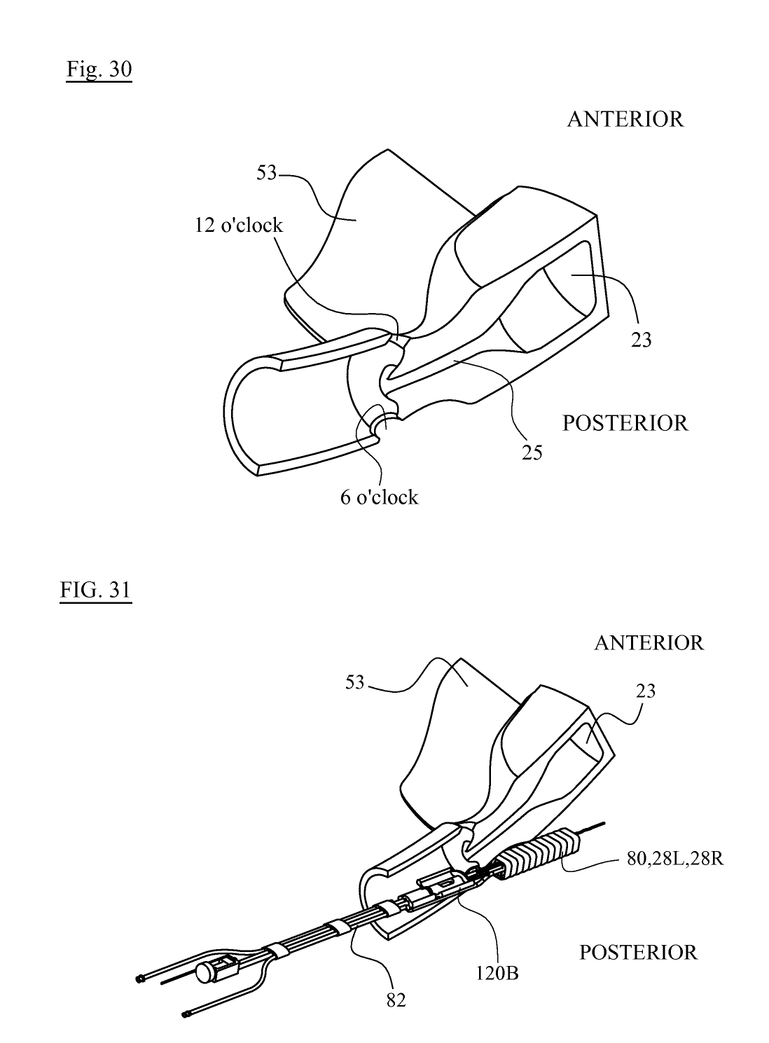

(5) A posterior colpotomy (i.e., an incision at approximately the 6 o'clock position) is performed, as shown in FIG. 30. For some applications, the posterior colpotomy site is identified by identifying the insertion of the uterosacral ligaments into the posterior aspect of the cervix. Responsive to the identification, an incision is made (e.g., using mayo scissors), thereby providing access to the peritoneal cavity.

(6) Balloons 28L and 28R are inserted through the posterior colpotomy, as shown in FIG. 31. At this stage, the balloons are typically positioned at the cul-de-sac, at the level of the uterosacral ligament insertion into the cervix, while the balloons are in a deflated, folded configuration, inside cover 80. The orientation of the balloons with respect to the subject's anatomy is typically adjusted at a later stage of the procedure, due to the insertion of first portion 120A of the fornix-engaging structure, and the coupling of first portion 120A with second portion 120B of the fornix-engaging structure.

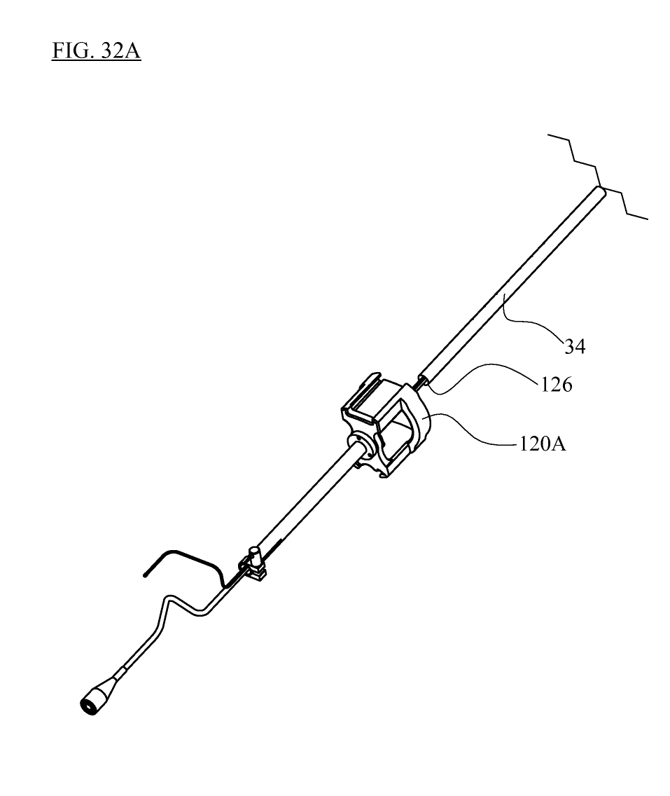

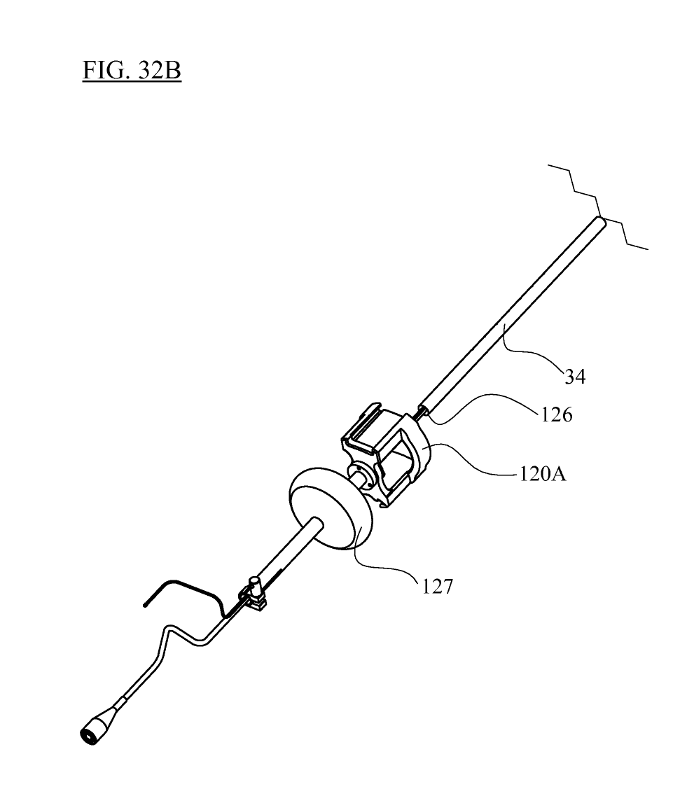

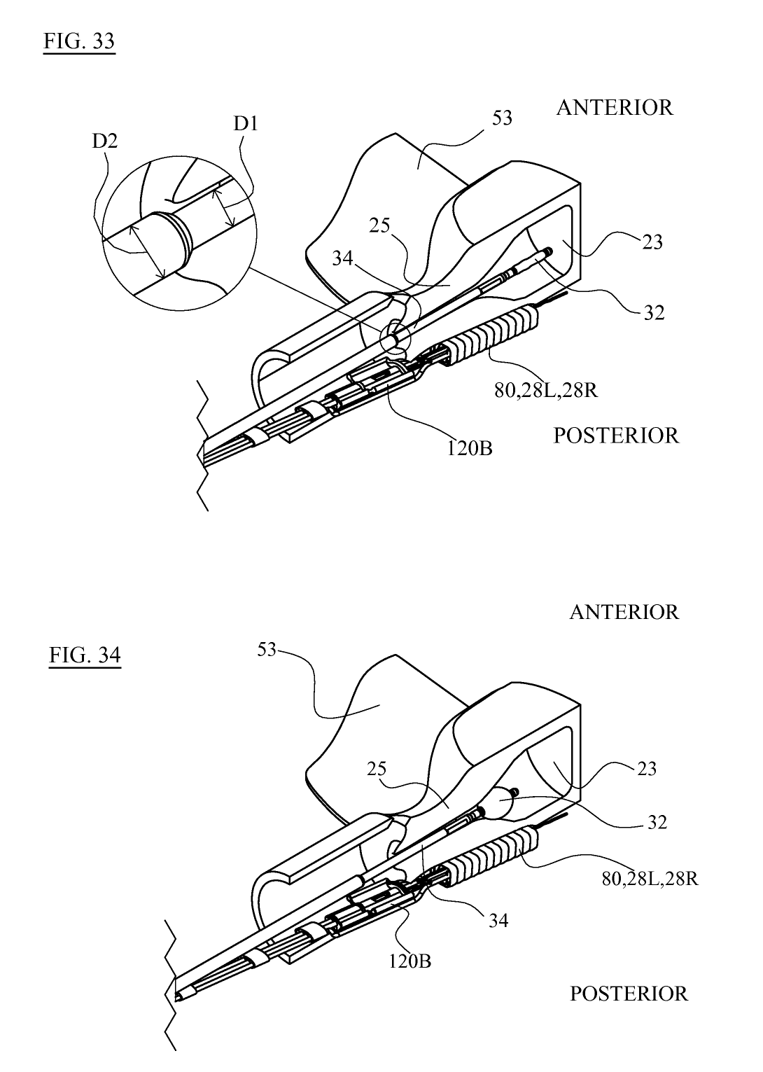

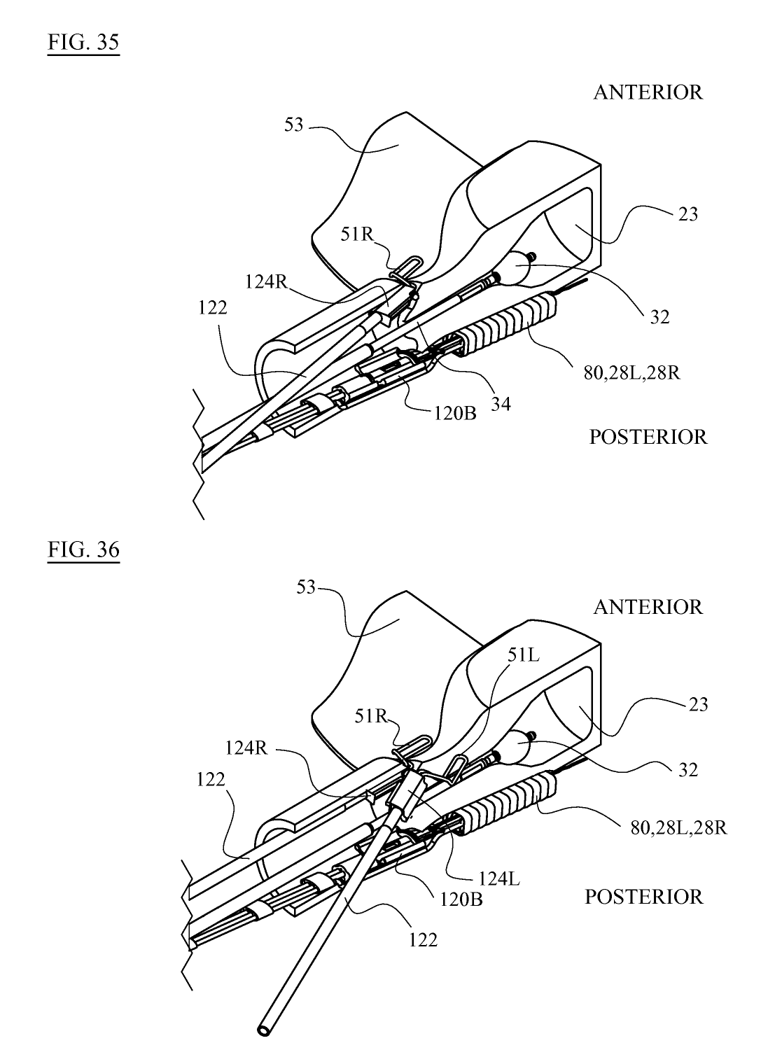

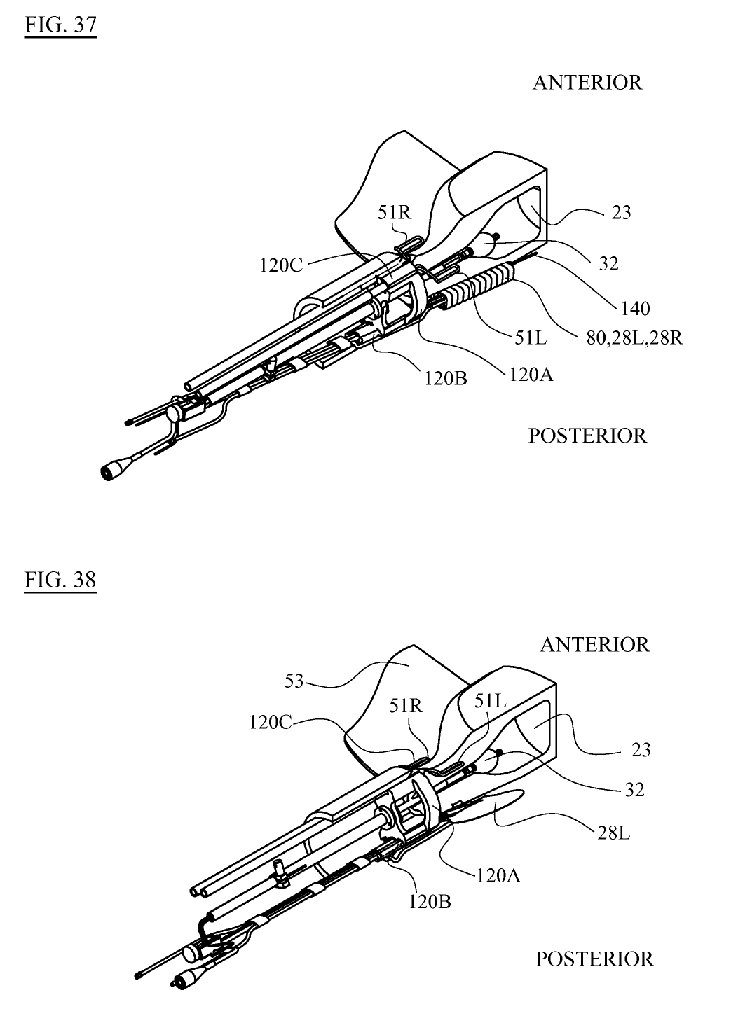

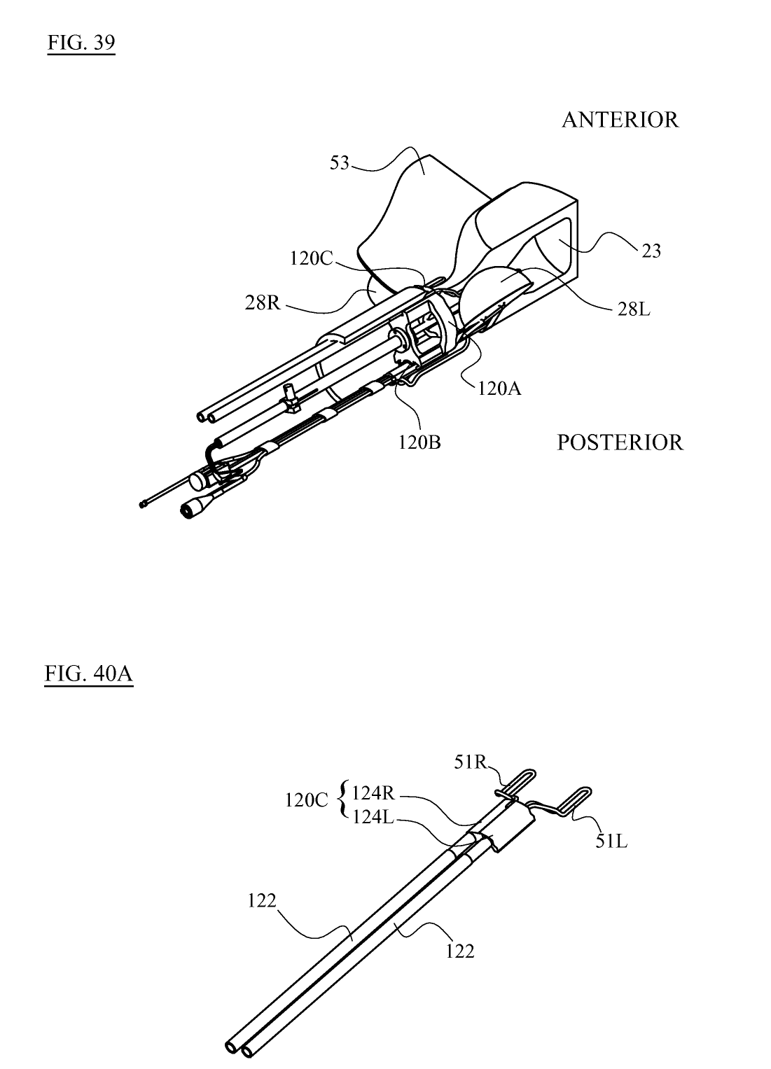

(7) Before inserting positioning-anchoring balloon 32 into the cervix, first portion 120A of fornix-engaging structure 120 is placed over positioning-anchoring tube 34, as shown in FIG. 32A. Typically, the aforementioned step is performed while portion 120A and positioning-anchoring balloon 32 are outside the subject's body. Subsequently, in order to facilitate insertion of the positioning-anchoring tube into the subject's vagina, portion 120A is slid proximally along the positioning-anchoring rod. For example, portion 120A may be slid to (or beyond) proximal end 126 of positioning-anchoring tube 34, for vaginal positioning at a later stage.

As shown in FIG. 32B, for some applications, an intravaginal balloon 127 is configured to stabilize portion 120A within the subject's vagina. In accordance with respective applications, intravaginal balloon 127 is used to stabilize portion 120A in addition to, or as an alternative to, positioning-anchoring balloon 34. Typically, the intravaginal balloon is coupled to positioning-anchoring tube 34, as shown. The intravaginal balloon is inflated inside the subject's vagina such as to expand against the walls of the vagina, thereby stabilizing portion 120A, which is coupled to the positioning-anchoring tube. In accordance with respective applications, intravaginal balloon 127 may be spherical, elliptical, and/or have a different shape, such as a figure-eight shape. For some applications, a plurality of (e.g., two) intravaginal balloons are used. In accordance with respective applications, the intravaginal balloon is composed of an elastic material, an inelastic material, or a combination of elastic and inelastic materials.

(8) Before inserting the positioning-anchoring tube inside the vagina, the length of the subject's uterus and cervical canal is determined, e.g., via ultrasound, or via sound.

(9) Positioning-anchoring tube 34 is inserted into the cervix (e.g., using a tenaculum), as shown in FIG. 33. It is noted that, as shown in FIG. 33, for some applications, a diameter D1 of a distal portion of positioning-anchoring tube 34 is less than a diameter D2 of a proximal portion of the positioning-anchoring tube. Similarly, the diameter of positioning-anchoring balloon 32, when positioning-anchoring balloon 32 is in a folded state thereof (as shown in FIG. 33), during insertion of the balloon through portion 120A of the fornix-engaging structure, is less than diameter D2 of the proximal portion of the positioning-anchoring tube. As noted hereinabove, for some applications, a balloon that is similar to the silicone balloon of The Rumi System® is used as the intrauterine balloon. Typically, the narrower diameter D1 of the distal portion of the tube 34 and of the balloon 32 facilitates insertion of the distal portion of the positioning-anchoring tube and the balloon through the subject's cervical canal. Further typically, the wider diameter D2 of the proximal portion of tube 34 facilitates accommodation of wiring and tubing by the proximal portion of the tube, and/or facilitates locking the proximal portion of the tube (e.g., via a clamp 154, shown in FIG. 42A) to portion 120A. For some applications, diameter D1 of the distal portion of tube 34 is more than 3 mm and/or less than 5 mm, e.g., 3-5 mm. For some applications, the diameter of positioning-anchoring balloon 32, when positioning-anchoring balloon 32 is in a folded state thereof, during insertion of the balloon through portion 120A of the fornix-engaging structure is more than 3 mm and/or less than 8 mm, e.g., 3-8 mm. For some applications, diameter D2 of the proximal portion of tube 34 is more than 4 mm, and/or less than 10 mm, e.g., 4-10 mm.

Subsequently, positioning-anchoring balloon 32 is inflated, as shown in FIG. 34. For some applications, the balloon is inflated to more than 200 mmHg, and/or less than 500 mmHg, e.g., 200-500 mmHg. Once the positioning-anchoring balloon has been inflated, the balloon is positioned in contact with the lower uterine segment, by pulling the positioning-anchoring tube 34 retrograde.

(10) Rigid structures 51L and 51R are inserted into the paracervical space via the anterior colpotomy, using insertion rods 122, as shown in FIGS. 35 and 36. Left and right support elements 124L and 124R are then coupled to each other. For some applications, insertion rods are subsequently decoupled from the support elements and removed from the subject's body. Alternatively, the insertion rods remain coupled to the support elements until the support elements are removed from the subject's body.

It is noted that as described with reference to FIGS. 35-36, for some applications, both left and right rigid structures 51 are inserted via a single vaginal incision at the 12 o'clock position. Alternatively, as described hereinabove (e.g., with reference to FIGS. 3A-F), for some applications incisions are made at approximately the 2 o'clock and 10 o'clock positions of the vaginal fornix, and the left and right rigid structures are inserted via respective, separate incisions. Typically, inserting both of the rigid structures via a single incision facilitates the creation of paracervical space above the cervix and the targeted arteries, with less risk to the arteries in the vicinity and/or to the ureters, relative to if the rigid structures are inserted via more than one incision. For some applications, this is because by making only one incision, the paracervical space may be created without the arteries and/or the ureters being repositioned.

For some applications, a blunt tool, and or a person's fingers are used to create the paracervical space. Typically, by using a blunt tool and/or a person's fingers, the risk of puncturing the subject's bladder, and/or damaging blood vessels of the subject is reduced, relative to if a sharp tool is used to create the paracervical space.

(11) First portion 120A of the fornix-engaging structure is inserted into the subject's fornix. The first portion is slid distally along the positioning-anchoring tube to the subject's fornix. When it is disposed at the subject's fornix, first portion 120A of the fornix-engaging structure is coupled to (a) second portion 120B and (b) third portion 120C of the fornix-engaging structure, as shown in FIG. 37. Typically, subsequent to coupling the portions of the fornix-engaging structure to each other, (a) positioning-anchoring tube 34 is pulled slightly retrograde, (b) fornix-engaging structure 120 is pushed such that the structure engages the fornix, and then (c) the position of the fornix-engaging structure with respect to the positioning-anchoring tube is locked, using a locking mechanism (e.g., a clamp, as described hereinbelow).

(12) At this stage, or at a different stage, monitoring of the sensor readings (e.g., readings of pressure sensors 48, microphones 46, and/or oximeters 40, as described hereinabove) is initiated.

(13) Cover 80 is removed from balloons 28L and 28R, e.g., by pulling string 140 (shown in FIG. 37), as described hereinbelow. The balloons are lifted anteriorly and are separated from each other by being moved respectively to left and right sides of the cervix (e.g., in accordance with the techniques described hereinabove), as shown in FIG. 38. Balloons 28L and 28R are inflated, in accordance with the techniques described hereinabove, as shown in FIG. 39. For some applications, the balloons are inflated to a pressure of more than 80 mmHg, and/or less than 300 mmHg, e.g., to 80-300 mmHg. The pressure of the balloons is typically displayed on a monitor of bed-side system 10.

(14) The output unit typically displays the following data, which are determined using the sensors described hereinabove:
(a) Pressure within balloons 28L and 28R, and pressure within positioning-anchoring balloon 32.
(b) Oxygen saturation, and/or blood pressure on right and left sides of the cervical canal.

The displayed data are typically monitored during the procedure. As described hereinabove, in response to the data, a healthcare professional, and/or control unit 52 modulates the pressure in balloons 28L, 28R, 55L, 55R, and/or 32.

(15) In response to the measured oxygen saturation, and/or the measured blood pressure reaching zero (or otherwise plateauing, as described hereinabove), balloons 28L and 28R are deflated, e.g., by opening stop cocks that are disposed in tubing that provides fluid communication between the balloons and the pressure-generating system. The apparatus is then typically removed from the subject's body in one or more of the following steps:
(a) Balloons 28L and 28R are folded and brought together, e.g., by turning knob 86, described hereinabove with reference to FIG. 13B.
(b) First portion 120A of the fornix-engaging structure is removed by unlocking the fornix-engaging structure from the positioning-anchoring tube, decoupling the first portion of the fornix-engaging structure from the second and third portions of the fornix-engaging structure, and pulling the first portion out of the subject's vagina.
(c) Balloons 28L and 28R are removed from the subject's body, using insertion assembly 82.
(d) Left and right support elements 124L and 124R of the rigid structures 51L and 51R are decoupled from one another and removed from the subject's body using insertion rods 122. The support elements are typically removed from the subject's body separately from one another.
(e) Positioning-anchoring balloon 32 is deflated (e.g., using a stop cock that is disposed in tubing that provides fluid communication between the balloon and the pressure-generating system). Subsequently, positioning-anchoring tube 34 is removed from the subject's body.

It is noted that, for some applications, not all of the procedure steps described with reference to FIGS. 26-39 are performed. It is further noted that the procedure steps described with reference to FIGS. 26-39 may be performed in an order different from that described hereinabove. It is still further noted that a generally similar procedure to that described with reference to FIGS. 26-39 may be performed using single portion fornix-engaging structure 24 or two-portion fornix-engaging structure 110, mutatis mutandis. In particular, it is noted that in the procedure shown in FIGS. 26-39, balloons 28 are shown as being uncovered and unfolded in the transition from FIG. 37 to FIG. 38. However, for some applications, the balloons are uncovered and unfolded subsequent to (e.g., immediately subsequently to) the step shown in FIG. 31, in which the balloons are inserted into the subject's abdomen through the posterior colpotomy. For such applications, the balloons typically remain uncovered, and unfolded, but uninflated, until after the step shown in FIG. 37 (in which the three portions of the fornix-engaging structure are coupled to one another) is performed, at which point the balloons are inflated, as shown in FIG. 39.

It is noted that rigid structure 51 may be viewed as defining pressure surfaces and support elements 124 may be viewed as pressure-conveying members configured to extend between the pressure surfaces and locations outside of the abdomen. First portion 120A of the fornix-engaging structure cooperates with the pressure-conveying members such that when the pressure-conveying members are seated against first portion 120A and removably coupled thereto, force is exerted from the pressure surface, through the pressure-conveying member, to first portion 120A. First portion 120A cooperates with the pressure-conveying member in order to bias the pressure surface toward an external surface of the uterine artery (and into engagement with tissue and/or a blood vessel) so as to at least partially occlude the uterine artery. Rods 84 to which balloons 28 are coupled may also be viewed as defining pressure surfaces and pressure-conveying members, which cooperate with first portion 120A in a similar manner.

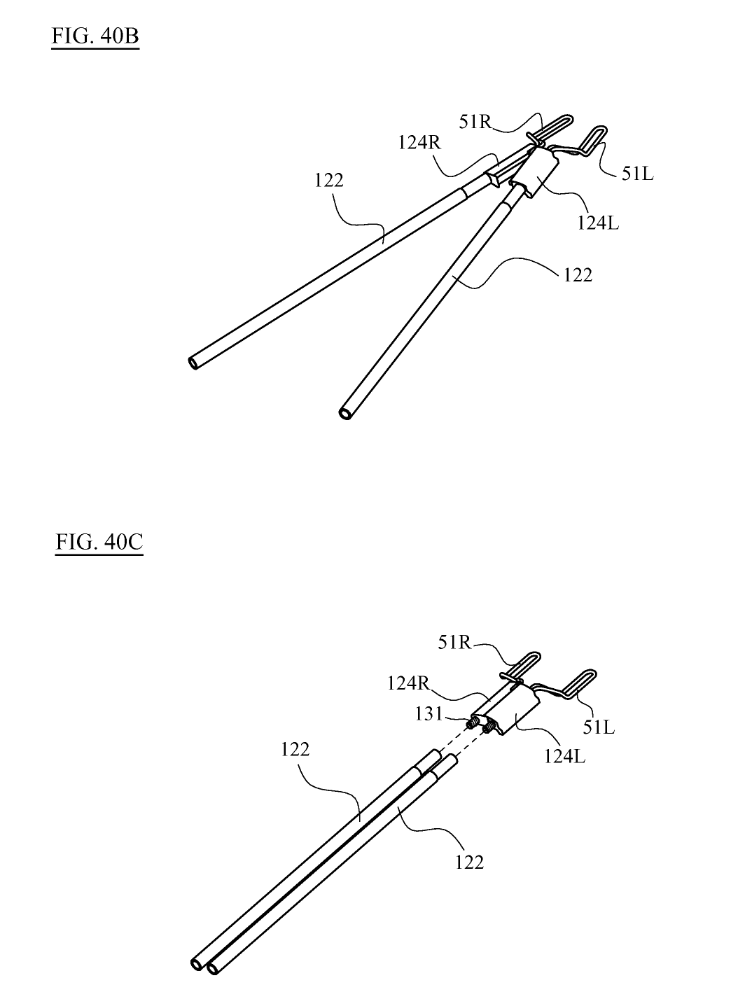

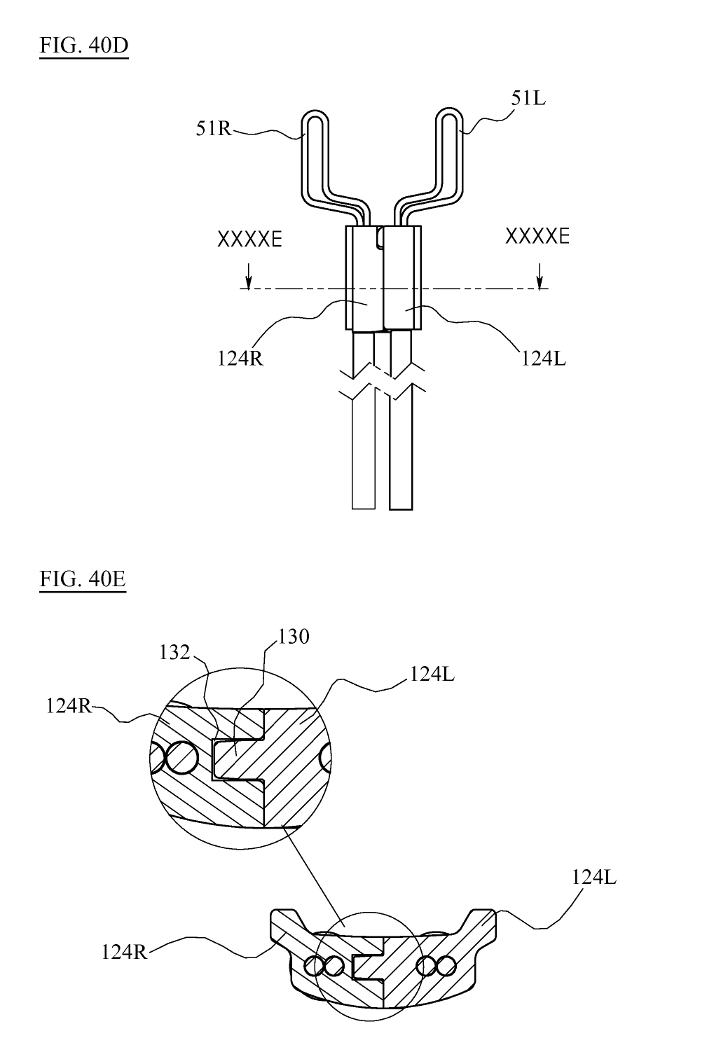

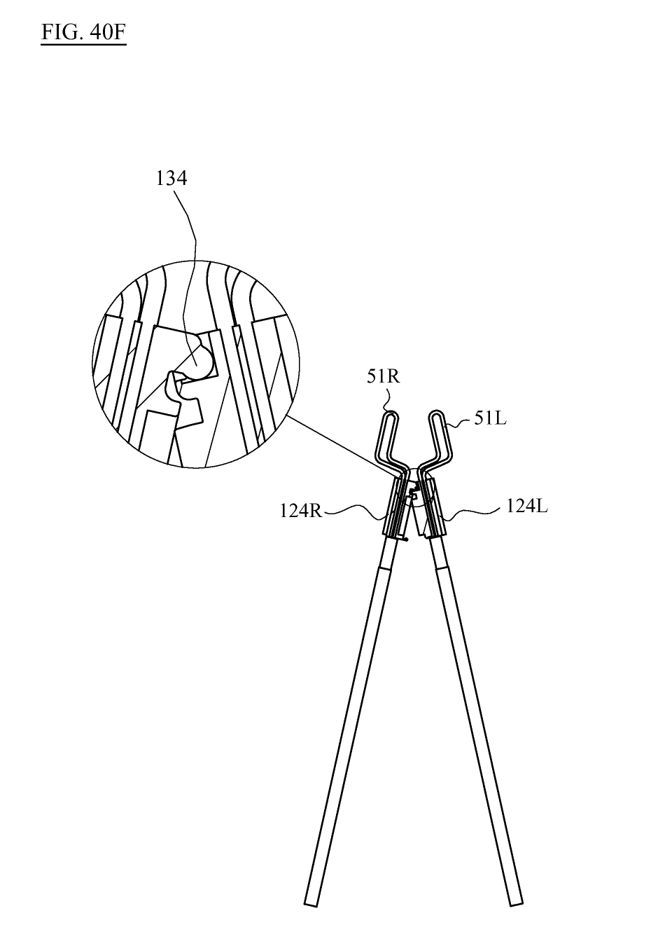

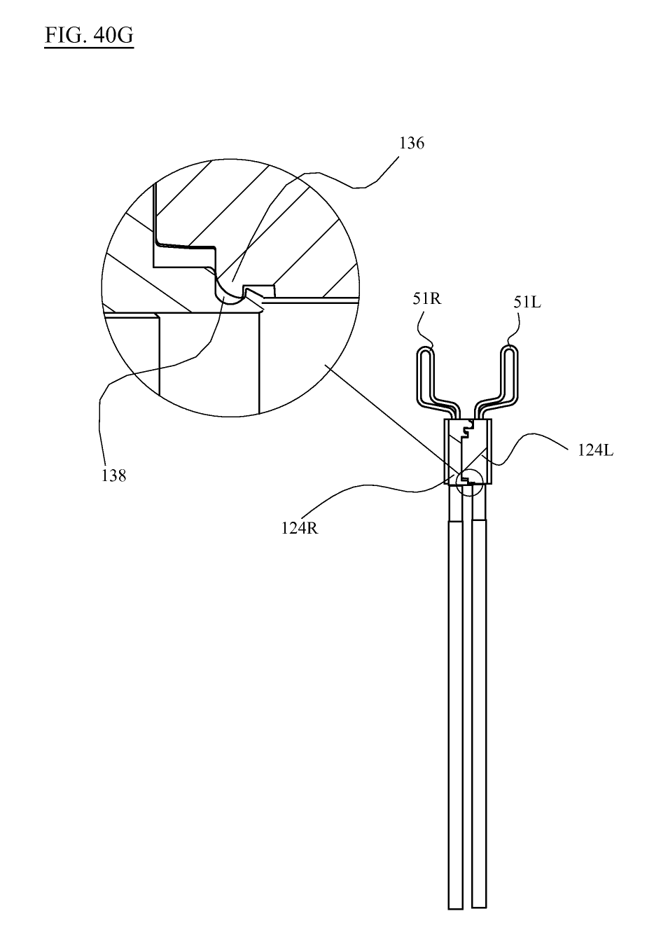

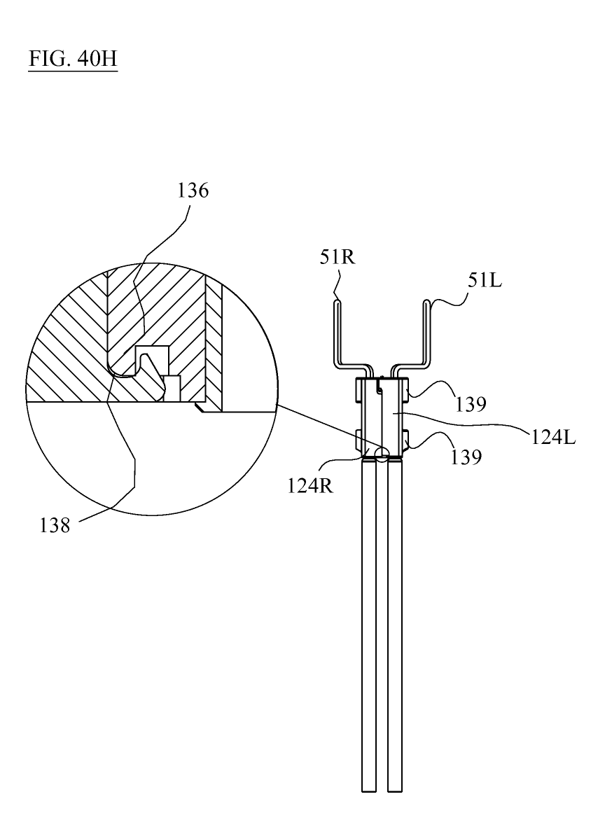

Reference is now made to FIGS. 40A-H, which are schematic illustrations of respective views of decouplable support elements 124L and 124R of non-penetrating rigid structures 51L and 51R, in accordance with some applications of the present invention. When coupled to each other, as shown in FIG. 40A, support elements 124L and 124R comprise third portion 120C of the three-portion fornix-engaging structure, described hereinabove with reference to FIG. 25. As shown in FIG. 40B, the support elements are decouplable from one another.

Insertion rods 122 are used to insert support elements 124L and 124R into the subject's fornix (and through the anterior incision in the fornix) and to remove the support elements from the subject's fornix. The insertion rods are typically decouplable from the support elements (e.g., via threading 131), as shown in FIG. 40C.

As shown in FIG. 40D-E, for some applications, left and right support elements 124L and 124R become coupled to each other (typically once they have passed through the anterior incision in the fornix) by a protrusion 130 on one of the support elements being inserted into a groove 132 on the other support element. Protrusion 130 is typically inserted into groove 132 by rotating one of the support elements with respect to the other support element on a hinge 134, as shown in FIG. 40F. When protrusion 130 is inserted inside groove 132, the support elements become held in place with respect to one another, for example, via a second protrusion 136 and a second groove 138, as shown in FIG. 40G.

For some applications, wings 139 protrude from the sides of left and right support elements 124L and 124R, as shown in FIG. 40H. Typically, a distal wing and a proximal wing is disposed on the side of each of the left and right support elements. The wings are used to couple the left and right support elements to first portion 120A of the fornix-engaging structure, as described hereinbelow with reference to FIG. 42C. Typically, the wings facilitate coupling of the left and right support elements to first portion 120A of the fornix-engaging structure, while reducing the relative movement between the support elements and the first portion of the fornix-engaging-structure that is required in order to perform the coupling. It is noted that although FIG. 40H shows two wings protruding from the sides of the support elements, for some applications a single wing protrudes from each one of the support elements, or more than two wings protrude from each of the support elements.

It is noted that the described coupling configuration is by way of illustration and not limitation. Alternative configurations include protrusions, grooves, or other coupling mechanisms being located at other locations with respect to left and right support elements 124L and 124R.

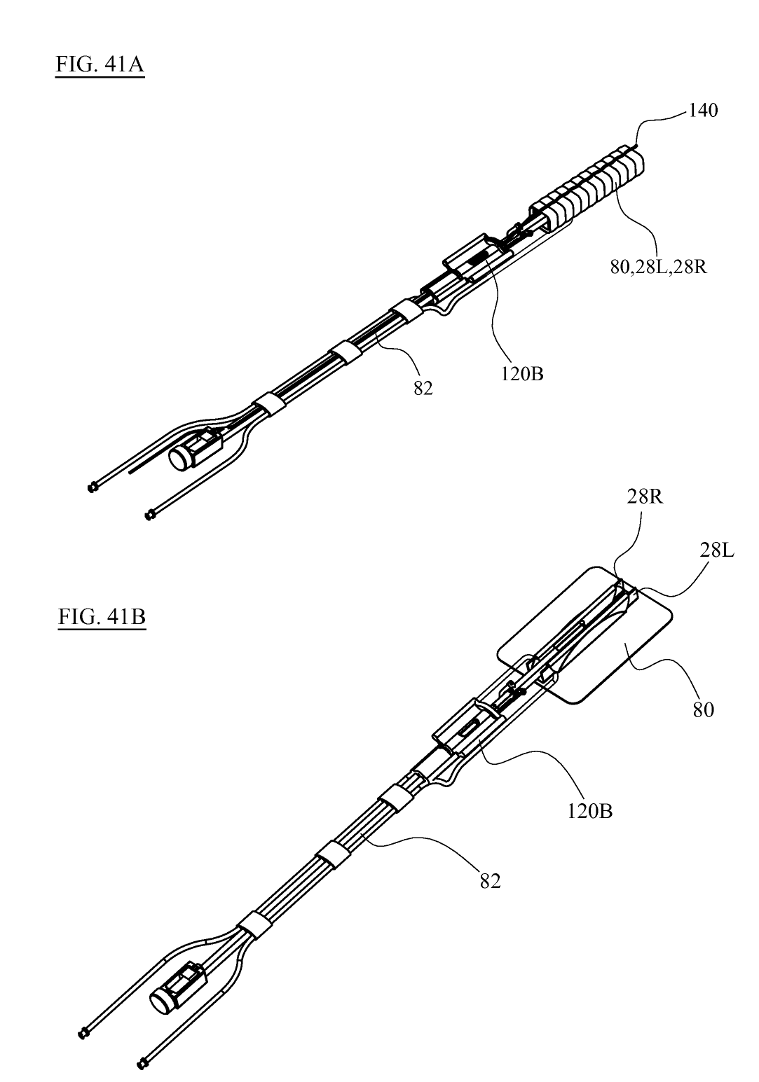

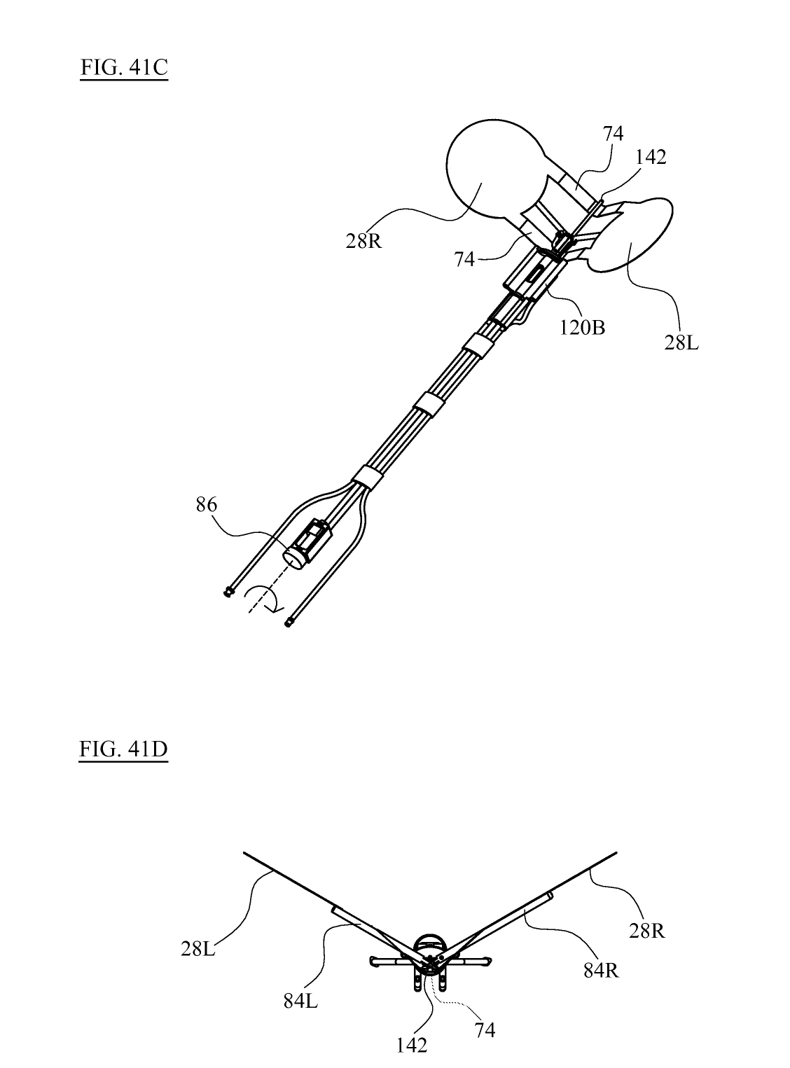

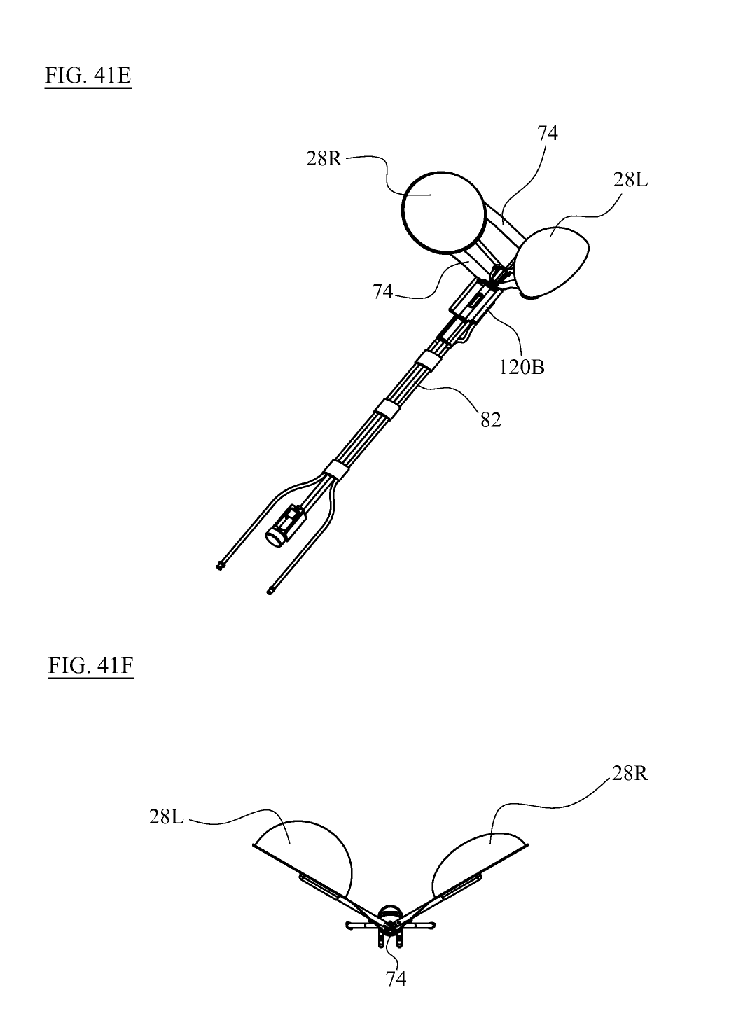

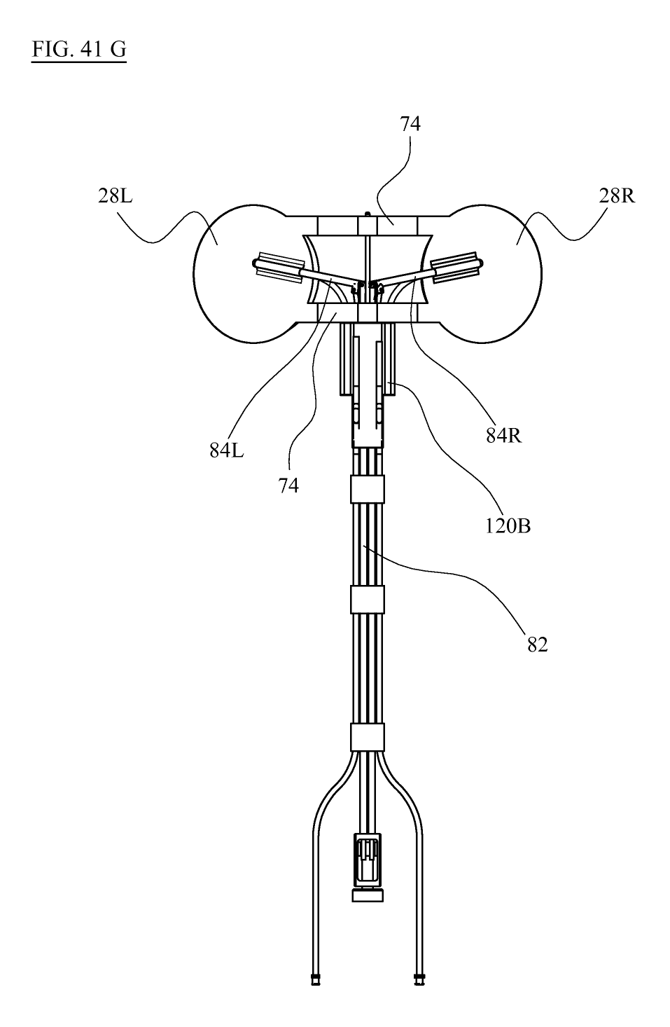

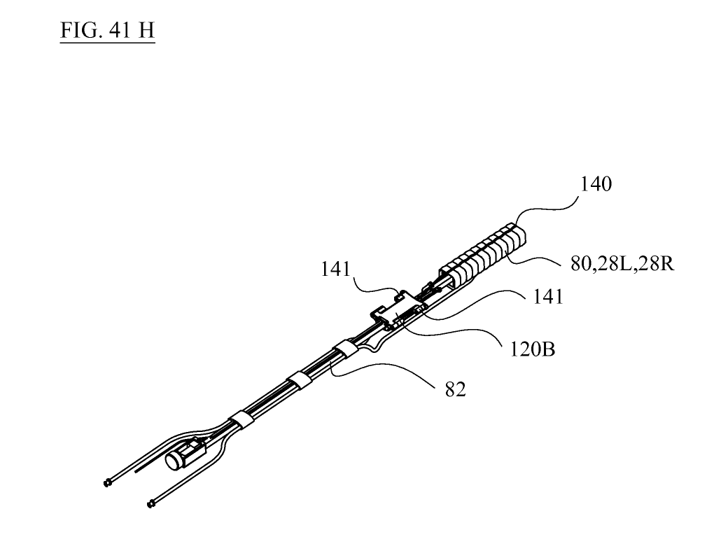

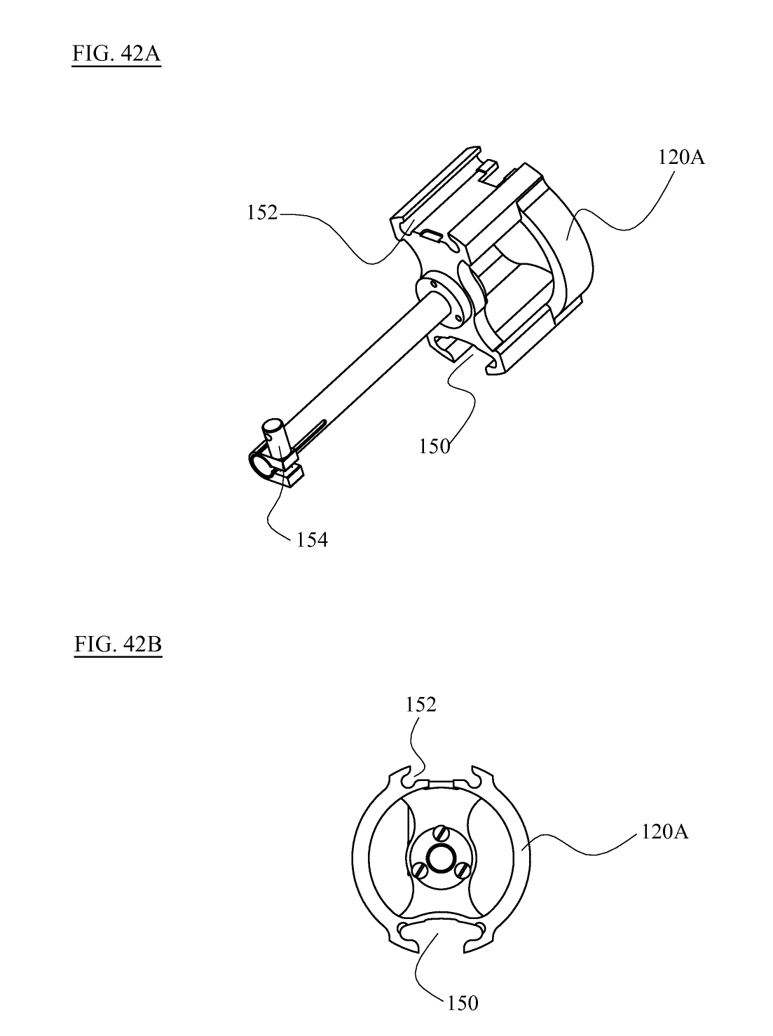

Reference is now made to FIGS. 41A-H, which are schematic illustrations of respective views of left and right balloons 28L and 28R that are used as at least a portion of a uterine artery compression device, in accordance with some applications of the present invention. Initially, the balloons are disposed inside cover 80, as shown in FIG. 41A. For some applications, the cover is removed from the balloons by pulling a thread 140, which causes the cover to open, as shown in FIG. 41B. Subsequent to the cover having been removed, the balloons are separated from one another and moved anteriorly, as described hereinabove. For some applications, the balloons are separated from one another and moved anteriorly by rotating knob 86 in a given direction, as shown in FIG. 41C, thereby moving left and right rods 84L, and 84R, as described hereinabove. (For some applications, in order to re-fold the balloons prior to the balloons being removed from the subject's body, knob 86 is rotated in the direction opposite to the given direction.)

As described hereinabove, the balloons are typically connected to one another via at least one flexible connecting member 74. As shown in FIG. 41C, for some applications, two or more connecting members connect the balloons. For some applications, the connecting members are longer than the distance between balloons 28L and 28R, even when the balloons are separated from one another. A rigid central rod 142 flexes the connecting members (FIG. 41C), causing the connecting members to maintain the balloons generally in the desired configuration shown in FIG. 41C, prior to inflation of the balloons. For some applications, rod 142 is coupled to the connecting members, for example, by an adhesive or via an additional portion (not shown) placed over rod 142. Typically, the disposition of the connecting members with respect to the rigid central rod additionally facilitates suitable positioning of the balloons with respect to one another, with respect to the subject's uterus, and/or with respect to other portions of device 20, during inflation of the balloons. FIG. 41D shows an end-view of the balloons in deflated states thereof, when the balloons have been lifted anteriorly and separated from one another.

Subsequent to balloons 28L and 28R being lifted anteriorly and separated from one another, the balloons are inflated. Respective views of the inflated balloons are shown in FIGS. 41E-G.

As shown in FIG. 41H, for some applications, wings 141 protrude from each side of second portion 120B of the fornix-engaging structure. Typically, a distal wing and a proximal wing is disposed on each side of the fornix-engaging structure. The wings are used to couple second portion 120B to first portion 120A of the fornix-engaging structure, as described hereinbelow with reference to FIG. 42C. Typically, the wings facilitate coupling of second portion of the fornix-engaging structure to first portion 120A of the fornix-engaging structure, while reducing the relative movement between the second portion and the first portion of the fornix-engaging-structure that is required in order to perform the coupling. It is noted that although FIG. 41H shows two wings protruding from each of the sides of the second portion of the fornix-engaging structure, for some applications a single wing protrudes from each of the sides of the second portion of the fornix-engaging structure, or more than two wings protrude from each of the sides of the second portion of the fornix-engaging structure.

Reference is now made to FIGS. 42A-C, which are schematic illustrations of a first portion 120A of three-portion fornix-engaging structure 120, in accordance with some applications of the present invention. First portion 120A defines a posterior slot 150, into which second portion 120B of the fornix-engaging structure is inserted. First portion 120A additionally defines an anterior slot 152, into which third portion 120C of the fornix-engaging structure is inserted. For some applications, portion 120A includes a clamp 154 at a proximal end thereof for locking positioning-anchoring tube 34 (not shown) with respect to portion 120A, in accordance with the techniques described hereinabove.

As shown in FIG. 42C, for some applications, first portion 120A of fornix-engaging structure 120 is shaped to define recesses 155. The recesses are shaped to accommodate the expansion of balloons 28L and 28R when the balloons are inflated, and to reduce any squeezing of cervical tissue and/or fornix tissue between first portion 120A and balloons 28L and 28R. Also, as shown in FIG. 42C, for some applications, first portion 120A of fornix-engaging structure 120 is shaped to define grooves 157 approximately midway along the length of anterior slot 152. The grooves are shaped to provide access to wings 139 of support elements 124L and 124 R (described hereinabove with reference to FIG. 40H). For some applications, in order to couple the support elements to the first portion of the fornix-engaging structure, the support elements are placed such that the proximal wings of the support elements are above grooves 157. The proximal wings are then inserted into the grooves and the support elements are slid proximally with respect to the first portion of the fornix-engaging structure (or the first portion of the fornix engaging structure is slid distally with respect to the support elements), such that the wings are held within anterior slot 152. Alternatively, in order to couple the support elements to the first portion of the fornix-engaging structure the support elements are placed such that the proximal wings of the support elements are distal to anterior slot 152. The proximal wings are then inserted into the distal end of slot 152 and the support elements are then slid proximally within the slot. It is noted that by inserting the proximal wings into grooves 157, the relative movement between the support elements and first-portion 120A that is required to couple the support elements to the first portion is reduced, relative to if the support elements are placed such that the proximal wings of the support elements are distal to anterior slot 152 in order to perform the coupling.

For some applications, first portion 120A of fornix-engaging structure 120 is shaped to define grooves (not shown) approximately midway along the length of posterior slot 150. The grooves are shaped to provide access to wings 141 of second portion 120B of the fornix-engaging structure (described with reference to FIG. 41H). The wings are used to couple second portion 120B to first portion 120A, in a similar manner to that described with reference to wings 139 of support elements 124L and 124R.

Reference is now made to FIGS. 42D-E, which are schematic illustrations of first portion 120A of three-portion fornix-engaging structure 120, in accordance with some applications of the present invention. For some applications, at least a portion of oximeter 40 (described hereinabove with reference to FIG. 7, for example) is disposed on fornix-engaging structure 120 (and/or a different fornix-engaging structure, as described herein). For example, as shown in FIG. 42D, one or more oximeter transmitters 76 (e.g., LED's) may be disposed on the inner surface of first portion 120A of the fornix-engaging structure, the LED's transmitting light to one or more oximeter receivers 78 that are disposed on positioning-anchoring tube 34. Alternatively, one or more oximeter transmitters (e.g., LED's) may be disposed on positioning-anchoring tube 34, the LED's transmitting light to one or more oximeter receivers that are disposed on the inner surface of the fornix-engaging structure (application not shown). In such configurations, the oximeter transmitters are configured to transmit light to the oximeter receivers via the fornix tissue and the cervical tissue, such that the receivers generate a signal indicative of capillary flow in the uterine arteries, the uterus, and/or the cervix by the oximeter detecting a level of oxyhemoglobin and/or deoxyhemoglobin in the uterine arteries, the uterus, and/or the cervix, and/or by detecting a pulse caused by blood flow in one or more of the aforementioned locations. As shown, for some applications, a first oximeter transmitter 76 (e.g., an LED) is disposed between the 12 o'clock and 3 o'clock positions (e.g., between the 1 o'clock and 2 o'clock positions) of the inner surface of the fornix-engaging structure; a second oximeter transmitter (e.g., an LED) is disposed between the 9 o'clock and 12 o'clock positions (e.g., between the 10 o'clock and 11 o'clock positions) of the inner surface of the fornix-engaging structure; and one or more oximeter receivers are disposed on positioning-anchoring tube 34.

For some applications, oximeter transmitters 76 and oximeter receivers 78 are disposed around the circumference of the inner surface of the fornix-engaging structure, as shown in FIG. 42E, for example. For example, oximeter transmitters and receivers may be disposed in a staggered configuration around the circumference of the inner surface of the fornix-engaging structure. By way of illustration and not limitation, a first oximeter transmitter 76 may be disposed between the 12 o'clock and 3 o'clock positions (e.g., between the 1 o'clock and 2 o'clock positions) of the inner surface of the fornix-engaging structure, and may transmit light to a first receiver 78 disposed between the 3 o'clock and 6 o'clock positions (e.g., between the 4 o'clock and 5 o'clock positions) of the inner surface of the fornix-engaging structure. Alternatively or additionally, a second oximeter transmitter 76 may be disposed between the 9 o'clock and 12 o'clock positions (e.g., between the 10 o'clock and 11 o'clock positions) of the inner surface of the fornix-engaging structure, and may transmit light to a second receiver 78 disposed between the 6 o'clock and 9 o'clock positions (e.g., between the 7 o'clock and 8 o'clock positions) of the inner surface of the fornix-engaging structure.

It is noted that although FIGS. 42D and 42E show oximeter transmitters 76 and oximeter receivers 78 disposed on first portion 120A of the fornix-engaging structure, for some applications at least one oximeter transmitter and/or oximeter receiver is disposed on second portion 120B, and/or third portion 120C of the fornix-engaging structure. Thus, in accordance with some applications, oximeter transmitter 76 may be disposed on positioning-anchoring tube 34, first portion 120A of the fornix engaging-structure, second portion 120B of the fornix engaging-structure, and/or third portion 120C of the fornix engaging-structure. Similarly, oximeter receiver 78 may be disposed on positioning-anchoring tube 34, first portion 120A of the fornix engaging-structure, second portion 120B of the fornix engaging-structure, and/or third portion 120C of the fornix engaging-structure.

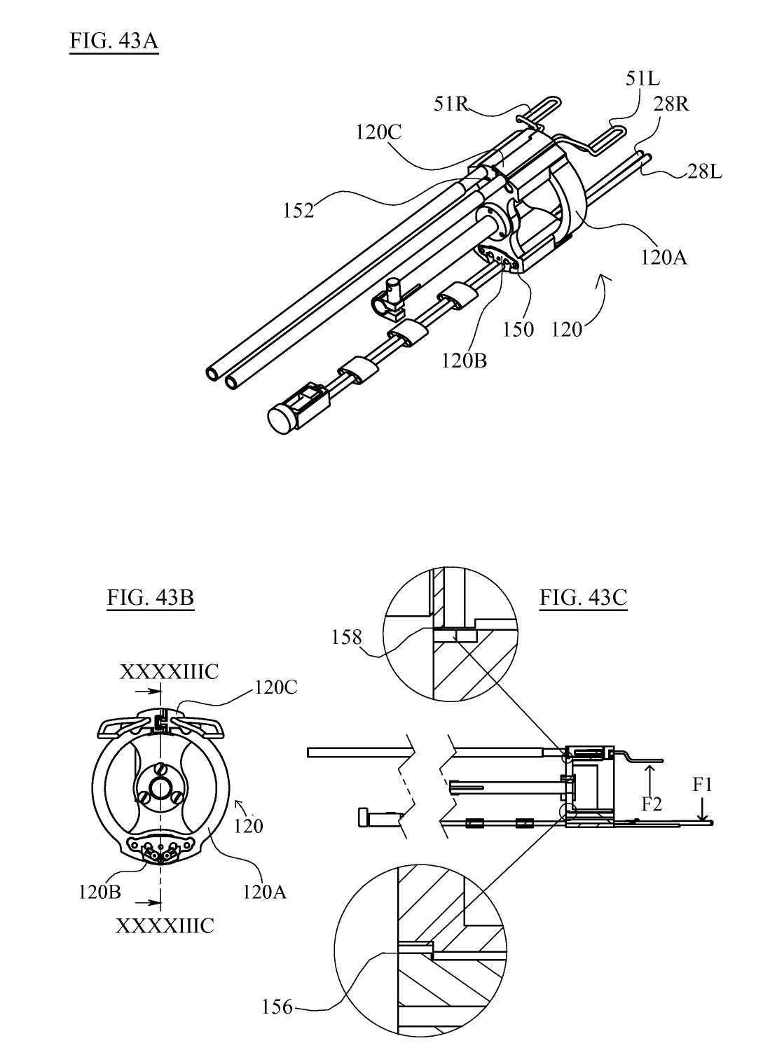

Reference is now made to FIGS. 43A-C, which are schematic illustrations of three-portion fornix-engaging structure 120, in accordance with some applications of the present invention. As shown, second portion 120B of the fornix-engaging structure is inserted into posterior slot 150 defined by first portion 120A of the fornix-engaging structure. The insertion of second portion 120B into posterior slot 150, typically couples second portion 120B to first portion 120A, such that the second portion is prevented from rotating with respect to the first portion. As shown in FIG. 43C, for some applications, a protrusion 156 from second portion 120B of the fornix-engaging structure prevents the second portion from sliding distally with respect to first portion 120A by the protrusion engaging with the first portion. Typically, protrusion 156 engages the first portion due to a downward force F1 that is applied to the second portion, causing the proximal end of the second portion to tilt upward. Force F1 is typically applied to the second portion during the procedure. Typically, when F1 is not applied to the second portion, the second portion of the fornix-engaging structure is able to slide distally with respect to the first portion.

As shown, third portion 120C of the fornix-engaging structure is inserted into anterior slot 152 defined by first portion 120A of the fornix-engaging structure. The insertion of second portion 120B into anterior slot 152, typically couples third portion 120C to first portion 120A, such that the third portion is prevented from rotating with respect to the first portion. As shown in FIG. 43C, for some applications, a protrusion 158 from third portion 120C of the fornix-engaging structure prevents the third portion from sliding distally with respect to first portion 120A by the protrusion engaging with the first portion. Typically, protrusion 158 engages the first portion due to an upward force F2 that is applied to the third portion, causing the proximal end of the third portion to tilt downward. Force F2 is typically applied to the third portion during the procedure. Typically, when F2 is not applied to the third portion, the third portion of the fornix-engaging structure is able to slide distally with respect to the first portion.

Reference is now made to FIGS. 44A-B, which are schematic illustrations of a device 20 for occluding uterine arteries, including one or more double-chambered balloons 160. Device 20 shown in FIGS. 44A-B is generally similar to the devices described hereinabove for occluding a subject's uterine arteries, except that the device includes double-chambered balloons 160 serving as left and right balloons 28L and 28R. Although balloons 160 are shown being used with three-portion fornix-engaging structure 120, the scope of the invention includes using balloons 160 with any of the fornix-engaging structures described herein. For some applications, using double-chambered balloons 160 facilitates expansion of the balloons in a desired direction during inflation of the balloons.

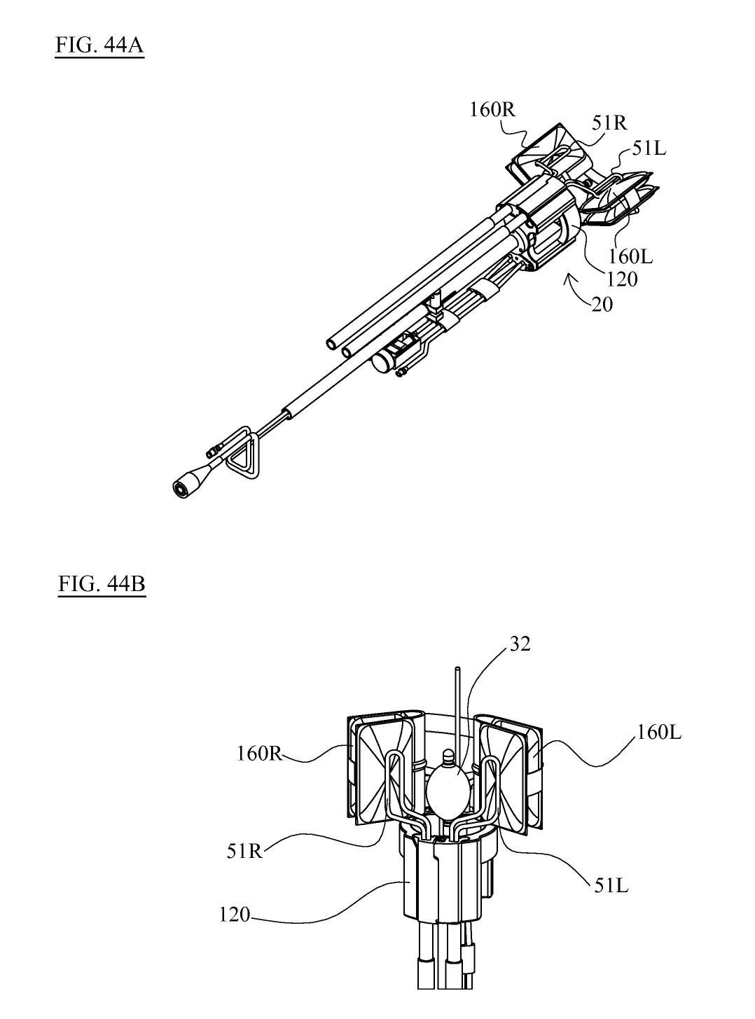

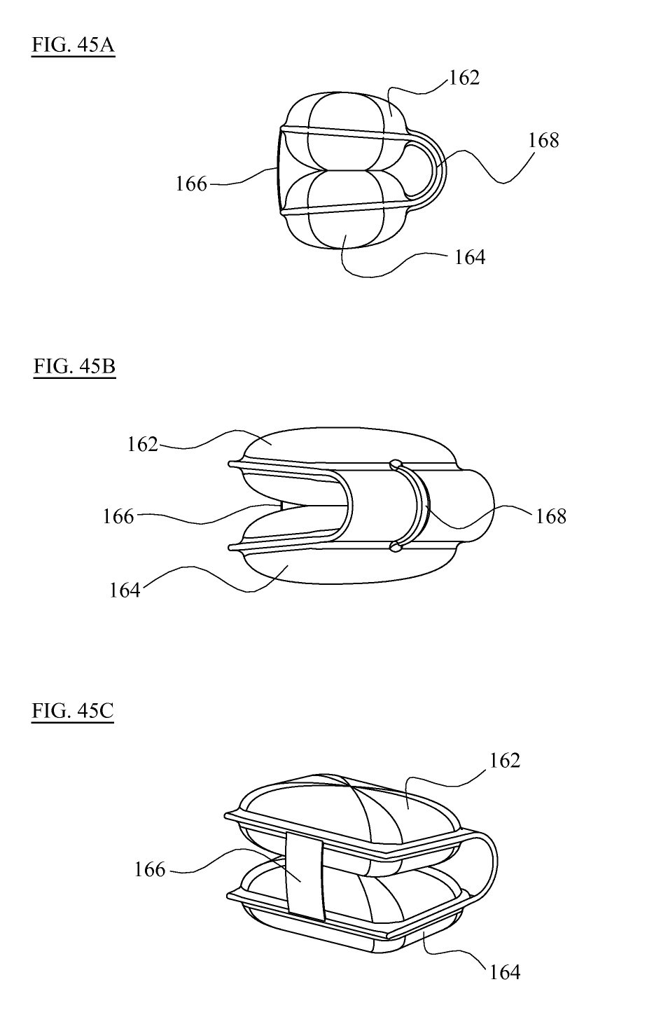

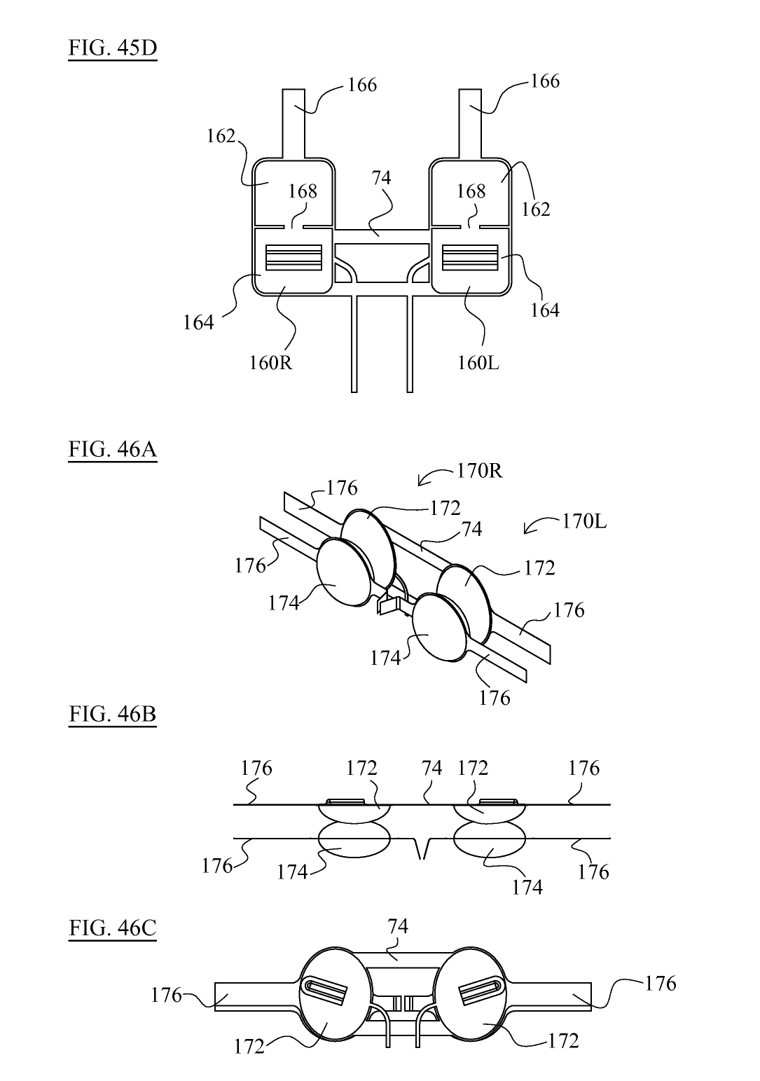

Reference is now made to FIGS. 45A-D, which are schematic illustrations of double-chambered balloon 160, in accordance with some applications of the present invention. FIGS. 45A-C show respective views of a single double-chambered balloon, while the double-chambered balloon is in an inflated state thereof. During use of the double-chambered balloons, first and second chambers 162 and 164 of the double-chambered balloon are folded with respect to each other, and are held in the folded configuration with respect to one another via a connecting element 166. As shown, the first and second chambers are in fluid communication with one another via a tube 168. In an alternative application, the first and second chambers are not in fluid communication with one another, and the chambers are inflated via separate inflation tubes.

FIG. 45D shows left and right double-chambered balloons 160L and 160R in uninflated, unfolded states. As described hereinabove, the left and right balloons are coupled to one another by one or more connecting members 74.

Reference is now made to FIGS. 46A-C, which are schematic illustrations of left and right double-chambered balloons 170L and 170R, in accordance with some applications of the present invention. Double-chambered balloons 170L and 170R typically serve as left and right balloons 28L and 28R, in accordance with the techniques described hereinabove. For some applications, using double-chambered balloons 170 facilitates expansion of the balloons in a desired direction (i.e., toward the subject's broad ligaments) during inflation of the balloons. The balloons typically expand in a manner that is similar to the expansion of a bellows. During use of the double-chambered balloons, first and second chambers 172 and 174 of each of the double-chambered balloons are coupled to one another at the centers of the chambers. For some applications, the first and second chambers of each of the balloons are in fluid communication with one another and are inflated via a single inflation tube. In an alternative application, the first and second chambers are not in fluid communication with one another, and the chambers are inflated via separate inflation tubes.

Typically, chambers 172 and 174 of each of balloons 170 are prevented from rotating with respect to one another due to the coupling of the chambers to one another at the centers of the chambers. For some applications, each of the chambers includes a connecting element 176. The connecting elements of each of the chambers of a balloon are connected to one another during inflation of the balloon, in order to prevent the chambers from rotating with respect to one another.

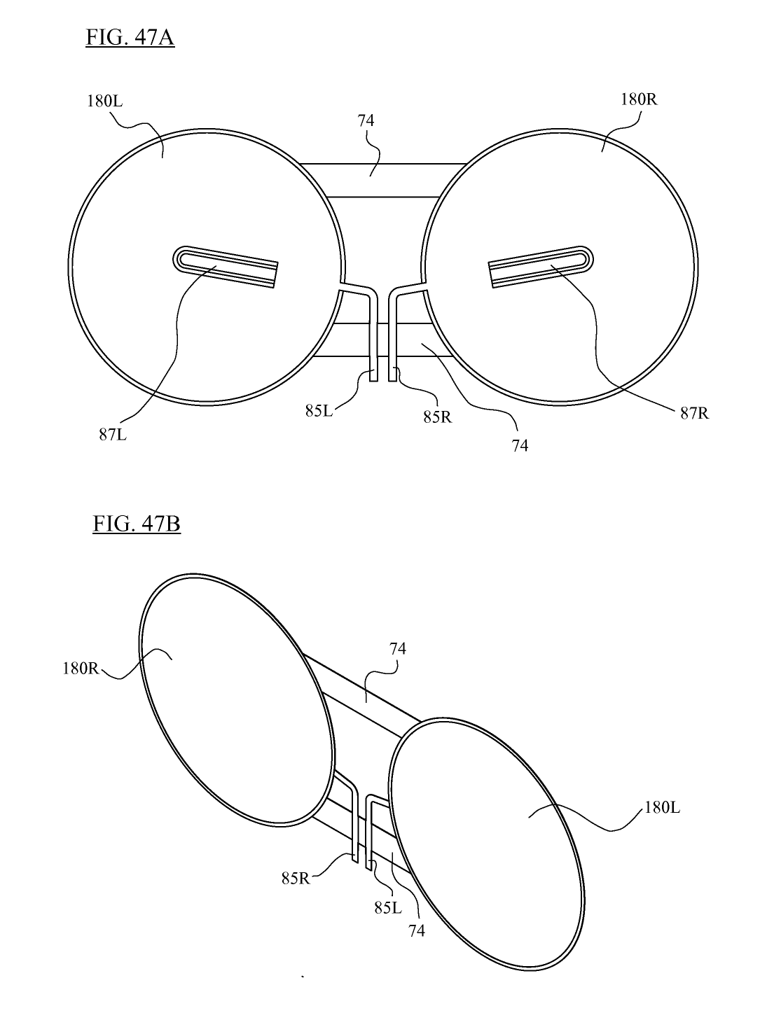

Reference is now made to FIGS. 47A-B, which are schematic illustrations of left and right single-chambered balloons 180L and 180R, in accordance with some applications of the present invention. Single-chambered balloons 180L and 180R typically serve as left and right balloons 28L and 28R, in accordance with the techniques described hereinabove. As described hereinabove, the left and right balloons are coupled to one another by one or more connecting members 74. For some applications, first and second connecting members connect the first and second balloons to one another. For some applications, as shown, the first and second connecting members are disposed between the left and right balloons, adjacent to, respectively, a distal portion of each of the balloons, and a proximal portion of each of the balloons. The balloons are typically inflated via inflation lumens 85L and 85R. For some applications, the balloons define sleeves 87L, 87R on posterior surfaces of the balloons. Left and right rods 84L and 84R (shown in FIG. 13A, for example) are coupled to balloons 28L and 28R by the rods being inserted into the sleeves.

It is noted that some embodiments described herein describe the placement of portions of a device (e.g. rigid structures 51) at a position anterior to a subject's broad ligaments. In the context of the present application, such descriptions include within their scope, the placement of the portions of the device anterior to a perivascular adipose layer of the subject's broad ligament, but, for example, posterior to the anterior-most mucosal layer of the subject's broad ligament.

Although applications are described herein relating to using a balloon as a uterine artery compression device, the scope of the present invention includes using other devices (e.g., solid or flexible devices) for occluding the uterine artery instead of or in addition to a balloon, mutatis mutandis. For example, a device that is configured to expand toward the subject's broad ligaments via an expansion mechanism, such as via a spring-mechanism, may be used as a uterine artery compression device.

Although applications are described herein relating to the compression and occlusion of a subject's uterine arteries, the scope of the present invention includes using the apparatus and methods described herein for occluding and/or compressing other portions of a subject's body, mutatis mutandis.

Although applications are described herein relating to the treatment of uterine fibroids, the scope of the present invention includes using the apparatus and methods described herein for treating endometriosis, post-partum hemorrhage, and/or adenomyosis, mutatis mutandis.

It is noted that although some applications of the present invention are described herein in the context of a transvaginal procedure, the scope of the present invention includes performing some or all of the procedure laparoscopically.

For some applications, the apparatus and techniques described herein are used in combination with apparatus and techniques described in (a) US 2009/0093758 to Gross (b) US 2009/0318950 to Gross, (c) US Patent Application 2011/0022073 to Gross, and/or (d) U.S. 61/300,262 to Gross, all of which applications are incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus, for use with a body, vagina, vaginal fornix, uterus, cervix, left and right broad ligaments and left and right uterine arteries of a subject, the apparatus comprising:
at least one rigid structure configured to be inserted into the subject's fornix such that distal end of the rigid structure is at a site anterior to a perivascular adipose layer of at least one of the subject's left and right broad ligaments; and
at least one uterine artery compression device configured to be placed posterior to the broad ligaments and to compress at least one of the subject's left and right uterine arteries by pressing the at least one broad ligament against the rigid structure.

2. The apparatus according to claim 1, wherein the at least one rigid structure comprises left and right rigid structures configured to be inserted into the subject's fornix such that distal ends of the rigid structures are at sites anterior to perivascular adipose layers of, respectively, the subject's left and right broad ligaments, and wherein the at least one uterine artery compression device is configured to compress the subject's left and right uterine arteries by pressing the subject's left and right broad ligaments against the rigid structures.

3. The apparatus according to claim 2, further comprising left and right rigid-structure-support-elements, the left and right rigid structures being disposed, respectively, on the left and right rigid-structure-support-elements, the rigid-structure-support-elements being reversibly couplable to one another.

4. The apparatus according to claim 2, further comprising:
at least one insertion assembly, the uterine artery compression device being disposed at a distal end of the insertion assembly; and
a fornix-engaging structure comprising first and second portions thereof,
the first portion of the fornix-engaging structure being coupled to the insertion assembly, and
the second portion of the fornix-engaging structure being configured to be couplable to the first portion of the fornix-engaging structure inside the subject's vagina.

5-8. (canceled)

9. The apparatus according to claim 2, further comprising an oximeter configured to be inserted via the subject's vagina and to measure a parameter of a portion of the subject's body selected from the group consisting of: the subject's uterus and the subject's cervix.

10. The apparatus according to claim 9, wherein the oximeter is configured to measure a level of oxyhemoglobin at the selected portion.

11. The apparatus according to claim 9, wherein the oximeter is configured to measure a pulse at the selected portion.

12. The apparatus according to claim 2,
further comprising a fornix-engaging structure configured to be inserted into the vagina and to engage the vaginal fornix,
wherein the left and right rigid structures are configured to be inserted into the subject's fornix via the fornix-engaging structure.

13. The apparatus according to claim 12, further comprising a locking-mechanism configured, subsequent to the insertion of the rigid structures via the fornix-engaging structure, to lock the rigid structures into a fixed position with respect to the fornix-engaging structure.

14. The apparatus according to claim 2, wherein the rigid structures comprise curved distal portions thereof.

15. The apparatus according to claim 14, wherein the rigid structures are not configured to incise tissue.

16-17. (canceled)

18. The apparatus according to claim 2, further comprising:
at least one insertion assembly, the uterine artery compression device being disposed at a distal end of the insertion assembly; and
a fornix-engaging structure comprising first, second, and third portions thereof,
the second portion of the fornix-engaging structure being coupled to the insertion assembly,
the third portion of the fornix-engaging structure being coupled to the rigid structures, and
the first portion of the fornix-engaging structure being configured to be couplable to the second and third portions of the fornix-engaging structure inside the subject's vagina.

19. The apparatus according to claim 18, wherein the second and third portions of the fornix-engaging structure are shaped to engage a region of the first portion of the fornix-engaging structure such that motion of the first and second portion in a distal direction with respect to the first portion is prevented.

20. The apparatus according to claim 18, wherein the third portion of the fornix-engaging structure comprises left and right rigid-structure-support-elements, the left and right rigid structures being disposed, respectively, on the left and right rigid-structure-support-elements, the rigid-structure-support-elements being reversibly couplable to one another.

21. The apparatus according to claim 18, further comprising an oximeter configured to be inserted via the subject's vagina and to measure a parameter of a portion of the subject's body selected from the group consisting of: the subject's uterus and the subject's cervix.

22-23. (canceled)

24. The apparatus according to claim 21, wherein the oximeter comprises an oximetry transmitter and an oximetry detector, and at least one of the oximetry receiver and the oximetry detector is coupled to the fornix-engaging structure.

25. The apparatus according to claim 2, wherein the uterine artery compression device comprises left and right balloons configured to be placed at positions posterior to, respectively, the left and right broad ligaments.

26. The apparatus according to claim 25, further comprising:
at least one connecting member, the balloons being coupled to one another via the connecting member; and
a rigid rod that is coupled to the balloons, and that is configured to maintain the balloons in a given configuration with respect to one another by flexing the connecting member.

27. (canceled)

28. The apparatus according to claim 1, further comprising a measuring device configured to measure a parameter that is indicative of a level of blood-flow through the subject's uterine arteries, and a control unit configured to generate an output that is indicative of the level of blood flow through the uterine arteries.

29. The apparatus according to claim 28, wherein the measuring device comprises a measuring device selected from the group consisting of an oximeter, a microphone, and a pressure sensor.

30. A method, for use with a body, vagina, vaginal fornix, uterus, cervix, left and right broad ligaments and left and right uterine arteries of a subject, the method comprising:
inserting at least one rigid structure into the subject's fornix such that distal end of the rigid structure is at a site anterior to a perivascular adipose layer of at least one of the subject's left and right broad ligaments;
placing at least one uterine artery compression device posterior to the broad ligaments; and
compressing at least one of the subject's left and right uterine arteries by pressing the at least one broad ligament against the rigid structures, using the uterine artery compression device.

31. The method according to claim 30, wherein inserting the at least one rigid structure comprises inserting left and right rigid structures such that distal ends of the rigid structures are at sites anterior to perivascular adipose layers of, respectively, the subject's left and right broad ligaments, and wherein compressing at least one of the subject's left and right uterine arteries comprises both of the subject's left and right uterine arteries by pressing the left and right broad ligaments against respective rigid structures, using the uterine artery compression device.

32. (canceled)

33. The method according to claim 31, wherein inserting the left and right rigid structures comprises inserting both the left and right rigid structures through a single incision in the subject's fornix.

34. The method according to claim 31, wherein inserting the left and right rigid structures comprises inserting the left and right rigid structures through respective incisions in the subject's fornix.

35-36. (canceled)

37. The method according to claim 31,
wherein placing the uterine artery compression device posterior to the broad ligaments comprises engaging the fornix with a second portion of a fornix-engaging structure, the uterine artery compression device being coupled to the second portion,
wherein inserting the left and right rigid structures into the subject's fornix comprises engaging the fornix with a third portion of a fornix-engaging structure, the rigid structures being coupled to the third portion of the fornix-engaging structure, and
further comprising, subsequent to the placement of the uterine artery compression device posterior to the broad ligaments and the insertion of the left and right rigid structures into the subject's fornix, coupling a first portion of the fornix-engaging structure to the second and third portions of the fornix-engaging structure, inside the vagina.

38. The method according to claim 31, further comprising measuring a level of oxygen in the subject's uterus, wherein compressing the subject's uterine arteries comprises compressing the uterine arteries responsively to the measured level of oxygen.

39. The method according to claim 31, wherein inserting the left and right rigid structures comprises inserting the left and right rigid structures into the subject's fornix via a fornix-engaging structure.

40. (canceled)

41. The method according to claim 39,
wherein placing the uterine artery compression device posterior to the broad ligaments comprises engaging the fornix with a first portion of the fornix-engaging structure, the uterine artery compression device being coupled to the first portion,
further comprising, subsequent to the placement of the uterine artery compression device posterior to the broad ligaments, coupling a second portion of the fornix-engaging structure to the first portion of the fornix-engaging structure, inside the vagina.

42-43. (canceled)

44. The method according to claim 31, wherein the uterine artery compression device includes left and right balloons, and wherein placing the uterine artery compression device posterior to the broad ligaments comprises placing the left and right balloons at positions posterior to, respectively, the left and right broad ligaments.

45. The method according to claim 44,
wherein the balloons include balloons that are coupled to one another via a connecting member; and
wherein the method further comprises maintaining the balloons in a given configuration with respect to one another by flexing the connecting member with a rigid rod that is coupled to the balloons.

46. (canceled)

47. The method according to claim 30, further comprising measuring a parameter that is indicative of a level of blood-flow through the subject's uterine arteries, and, responsively thereto, generating an output that is indicative of the level of blood-flow through the uterine arteries.

48. The method according to claim 47, wherein measuring the parameter comprises measuring the parameter using a measuring device selected from the group consisting of an oximeter, a microphone, and a pressure sensor.

49-230. (canceled)
